(19) United States
(12) Patent Application Publication
(10) Pub. No.: US 2005/0004186 A1
(43) Pub. Date: Jan. 6, 2005

(54) MEK inhibiting compounds

(75) Inventors: Stephen D. Barrett, Hartland, MI (US); Cathlin M. Flamme, Lawrenceville, NJ (US); Michael D. Kaufman, Ypsilanti, MI (US); Mark S. Plummer, Dexter, MI (US); Jessica E. Reed, Ann Arbor, MI (US); Joseph S. Warmus, Ann Arbor, MI (US); Lu-Yan Zhang, Branford, CT (US)

Correspondence Address: WARNER-LAMBERT COMPANY, 2800 PLYMOUTH RD, ANN ARBOR, MI, 48105, US

(73) Assignee: Pfizer Inc

(21) Appl. No.: 10/725,206

(22) Filed: Dec. 1, 2003

Related U.S. Application Data
(60) Provisional application No. 60/509,701, filed on Oct. 8, 2003.
(60) Provisional application No. 60/435,155, filed on Dec. 20, 2002.

(51) Int. Cl. C07D 285/14; C07D 271/12; C07D 249/08; A61K 31/433; A61K 31/4245; A61K 31/4196

(52) U.S. Cl. 514/363; 514/364; 514/383; 548/136; 548/143; 548/269.4

(57) ABSTRACT

This invention provides substituted Phenyl-(2-[1,3,4]thiadiazol-2-yl-phenyl)-amine and (2-[1,3,4]Oxadiazol-2-yl-phenyl)phenyl-amine compounds which act as inhibitors of MAPK/ERK Kinase (“MEK”) enzymes and pharmaceutical compositions and methods for their use in immunomodulation and in the treatment and alleviation of inflammation, and proliferative diseases such as cancer and restenosis.

This application is a United States utility application which claims the benefit of priority to U.S. provisional application Ser. No. 60/509,701 filed Oct. 8, 2003 and U.S. provisional application Ser. No. 60/435,155 filed Dec. 20, 2002.

### BACKGROUND OF THE INVENTION

MAPK/ERK Kinase (“MEK”) enzymes are dual specificity kinases involved in, for example, immunomodulation, inflammation, and proliferative diseases such as cancer and restenosis.

Proliferative diseases are caused by a defect in the intracellular signaling system, or the signal transduction mechanism of certain proteins. Defects include a change either in the intrinsic activity or in the cellular concentration of one or more signaling proteins in the signaling cascade. The cell may produce a growth factor that binds to its own receptors, resulting in an autocrine loop, which continually stimulates proliferation. Mutations or overexpression of intracellular signaling proteins can lead to spurious mitogenic signals within the cell. Some of the most common mutations occur in genes encoding the protein known as Ras, a G-protein that is activated when bound to GTP, and inactivated when bound to GDP. The above-mentioned growth factor receptors, and many other mitogenic receptors, when activated, lead to Ras being converted from the GDP-bound state to the GTP-bound state. This signal is an absolute prerequisite for proliferation in most cell types. Defects in this signaling system, especially in the deactivation of the Ras-GTP complex, are common in cancers. and lead to the signaling cascade below Ras being chronically activated.

Activated Ras leads in turn to the activation of a cascade of serine/threonine kinases. One of the groups of kinases known to require an active Ras-GTP for its own activation is the Raf family. These in turn activate MEK (e.g., MEK₁ and MEK₂) which then activates the MAP kinase, ERK (ERK₁ and ERK₂). Activation of MAP kinase by mitogens appears to be essential for proliferation; constitutive activation of this kinase is sufficient to induce cellular transformation. Blockade of downstream Ras signaling, for example by use of a dominant negative Raf-1 protein, can completely inhibit mitogenesis, whether induced from cell surface receptors or from oncogenic Ras mutants. Although Ras is not itself a protein kinase, it participates in the activation of Raf and other kinases, most likely through a phosphorylation mechanism. Once activated, Raf and other kinases phosphorylate MEK on two closely adjacent serine residues, S²¹⁸ and S²²² in the case of MEK-1, which are the prerequisite for activation of MEK as a kinase. MEK in turn phosphorylates MAP kinase on both a tyrosine, Y¹⁸⁵, and a threonine residue, T¹⁸³, separated by a single amino acid. This double phosphorylation activates MAP kinase at least 100-fold. Activated MAP kinase can then catalyze the phosphorylation of a large number of proteins, including several transcription factors and other kinases. Many of these MAP kinase phosphorylations are mitogenically activating for the target protein, such as a kinase, a transcription factor, or another cellular protein. In addition to Raf-1 and MEKK, other kinases activate MEK, and MEK itself appears to be a signal integrating kinase. Current understanding is that MEK is highly specific for the phosphorylation of MAP kinase. In fact, no substrate for MEK other than the MAP kinase, ERK, has been demonstrated to date and MEK does not phosphorylate peptides based on the MAP kinase phosphorylation sequence, or even phosphorylate denatured MAP kinase. MEK also appears to associate strongly with MAP kinase prior to phosphorylating it, suggesting that phosphorylation of MAP kinase by MEK may require a prior strong interaction between the two proteins. Both this requirement and the unusual specificity of MEK are suggestive that it may have enough difference in its mechanism of action to other protein kinases that selective inhibitors of MEK, possibly operating through allosteric mechanisms rather than through the usual blockade of the ATP binding site, may be found.

It has been found that the compounds of the present invention are inhibitors of MEK and are useful in the treatment of a variety of proliferative disease states, such as conditions related to the hyperactivity of MEK, as well as diseases modulated by the MEK cascade.

### SUMMARY OF THE INVENTION

This invention comprises compounds of the formula: 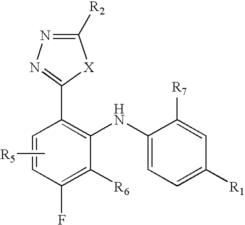
wherein:

X is NH, O or S;

R₁ is selected from halogen, —C═CH₂, —O—C₁-C₆ alkyl, —C═CH—(CH₂)ₘ—O—C₁-C₆ alkyl, —C═C—(CH₂)ₘ—O—C₁-C₆ alkyl, —C═C—(CH₂)ₘ—NH—C₁-C₆ alkyl, —C≡CH, —C≡C—(CH₂)ₘ—OH, C≡C—(CH₂)ₘ—NH₂,—COOH, —(CH₂)ₘ—COOH, —CONH₂, —COO—C₁-C₆ alkyl, —(CH₂)ₘ—CONH₂, —C(O)(C₁-C₆ alkyl), —C(O)NHSO₂H, —(CH₂)ₘ—C(O)NHSO₂H, —C(O)NHSO₂—(C₁-C₃ alkyl), —(CH₂)ₘ—C(O)NHSO₂—(C₁-C₃ alkyl), or C₁-C₆ alkyl, —S—C₁-C₆ alkyl or alkenyl, with the alkyl, —S-alkyl and alkenyl chains in each of these R₁ moieties being optionally substituted by from 1 to 3 OH groups and/or by from 1 to 5 fluorine atoms;

R₂ is —R₃, —O—R₃, —S—R₃ or a moiety selected from the group of NH₂, NHR₃, N(C₁-C₃ alkyl)-R₃, —NH—(CH₂)ₙ—O—(C₁-C₃ alkyl) or —N((CH₂)ₙ—O—(C₁-C₃ alkyl))₂, or a moiety selected from the group of:

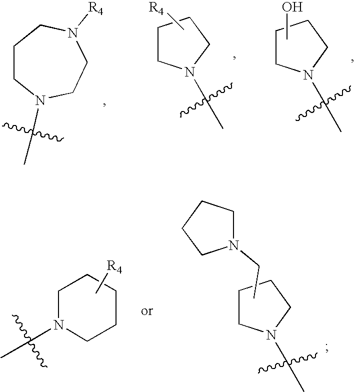

R₃ is a moiety selected from:
a) C₁-C₈ alkyl, C₁-C₈ alkenyl, the alkyl and alkenyl groups each being optionally substituted by from 1 to 4 OH or halogen groups;
b) —(CH₂)ₙ—NH₂, —(CH₂)ₙ—NH(C₁-C₃ alkyl), —(CH₂)ₙ—N(C₁-C₃ alkyl)₂, —(CH₂)ₙ—O—(C₁-C₃ alkyl), —(CH₂)ₘ—C(O)—O—(C₁-C₃ alkyl), or (CH₂)ₙ—N(C₁-C₃ alkyl)—C(O)—(C₁-C₃ alkyl);
c) phenyl, —(CH₂)ₘ-phenyl, —(CH₂)ₘ—O-phenyl, the phenyl ring of each being optionally substituted by 1 or 2 groups selected from C₁-C₃ alkoxy, NH₂, NH(C₁-C₃ alkyl), N(C₁-C₃ alkyl)₂; or
d) a moiety selected from the group of:

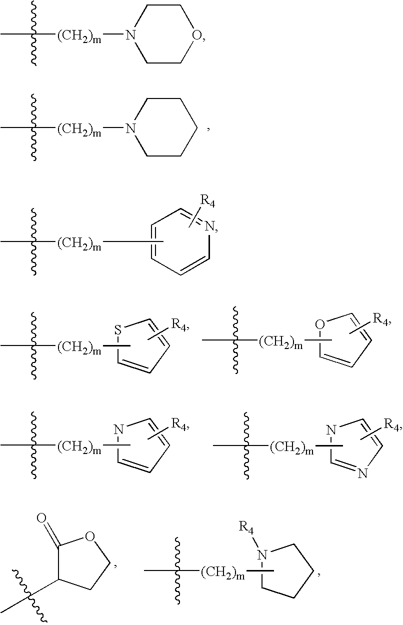

R₄ is selected from H or C₁-C₃ alkyl, optionally substituted by OH;

R₅ is selected from H or halogen;

R₆ is selected from H or F;

R₇ is selected from F, CH₂F, CHF₂, or —CF₃;

n in each instance is independently selected as an integer of from 1 to 6

m in each instance is independently selected as an integer of from 1 to 4;

or a pharmaceutically acceptable salt or ester form thereof.

In the definition of R₃ above, the term C₁-C₈ alkyl is understood to include straight chain, branched or cyclic alkyl groups, as well as combination thereof. These groups include cyclic and bridged cyclic alkyl groups such as cyclopropyl, cyclobutyl, cyclopentyl, cyclohexyl, cycloheptyl, cycloooctyl, bicyclo[2.2.1]heptanyl, etc., each optionally linked by an alkyl chain. Unless otherwise indicated, halogen is understood to indicate F, I, Cl or Br.

One group of compounds of this invention comprises those of the formula above wherein X is O. A subgroup of these compounds includes those of the formula: 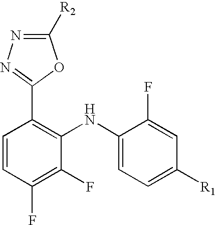
wherein:

R₁ is selected from I, Br or C₁-C₃ alkyl;

R₂ is R₃, —O—R₃, —S—R₃ or a moiety selected from the group of NH₂, NHR₃, N(C₁-C₃ alkyl)-R₃, —NH—(CH₂)ₙ—O—(C₁-C₃ alkyl), —NH—(CH₂)ₙ—O—C(O)—(C₁-C₃ alkyl) or —N((CH₂)ₙ—O—(C₁-C₃ alkyl))₂;

R₃ is a moiety selected from:
a) C₁-C₈ alkyl, C₁-C₈ alkenyl, the alkyl and alkenyl groups each being optionally substituted by from 1 to 4 OH groups;
b) —(CH₂)ₙ—NH₂, —(CH₂)ₙ—NH(C₁-C₃ alkyl), —(CH₂)ₙ—N(C₁-C₃ alkyl)₂, —(CH₂)ₙ—O—(C₁-C₃ alkyl), (CH₂)ₘ—C(O)—O—(C₁-C₃ alkyl),
c) phenyl, —(CH₂)ₘ-phenyl, —(CH₂)ₘ—O-phenyl, the phenyl ring of each being optionally substituted by 1 or 2 groups selected from C₁-C₃ alkoxy, NH₂, NH(C₁-C₃ alkyl), N(C₁-C₃ alkyl)₂; or
d) a moiety selected from the group of:

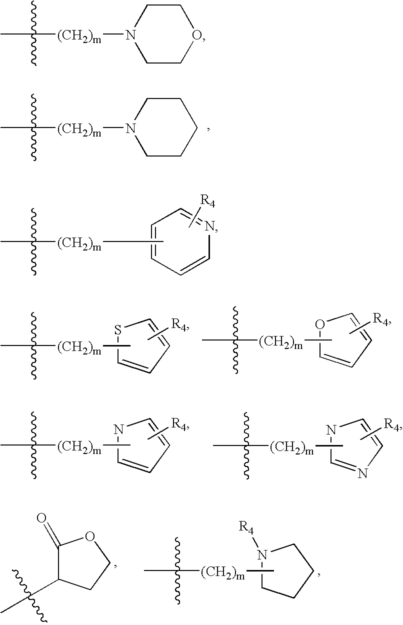

R₄ is selected from H or C₁-C₃ alkyl, optionally substituted by OH;

n is an integer of from 1 to 6

m is an integer of from 1 to 4;

or a pharmaceutically acceptable salt form thereof.

Within each of the groups described herein is a subset of compounds in which R₁ is F or Br. A further subset of compounds comprises those in which R₁ is F.

Pharmaceutically or therapeutically useful esters of this invention include carboxylic acid ester in which the non-carbonyl moiety of the ester group is selected from straight, branched, or cyclic alkyl, alkenyl, alkynyl, alkoxyalkyl including methoxymethyl, aralkyl including benzyl or phenethyl groups, aryloxyalkyl such as phenoxymethyl, aryl including phenyl and naphthyl groups, optionally substituted with halogen, C₁ to C₆ alkyl or C₁ to C₆ alkoxy, sulfonate esters such as alkyl or aralkyl sulphonyl including methanesulfonyl, the mono, di or triphosphate ester, trityl or monomethoxytrityl, substituted benzyl, trialkylsilyl (e.g. dimethyl-t-butylsilyl) or diphenylmethylsilyl. Aryl groups in the esters optimally comprise a phenyl group. The alkyl group can be straight, branched, or cyclic, and is optimally a C₁ to C₁₈ group. Examples of straight chain or branched C₁-C₁₈ alkyl esters include methyl, ethyl, propyl, isopropyl, butyl, isobutyl, amyl, hexyl, heptyl, octyl, nonyl, decyl, lauryl, myristyl, cetyl, and stearyl, etc. Straight chain or branched C₂-C₁₈ alkenyl esters include vinyl, allyl, undecenyl, oleyl, and linolenyl esters, etc.

Examples of cyclic alkyl groups include cyclopropyl, cyclobutyl, cyclopentyl, cyclohexyl, cycloheptyl, and cyclooctyl groups. Cycloalkyl groups can also include bridged carbocyclic groups, such as a bicyclo[2.2.1]heptanyl group. Also useful are alkyl-cycloalkyl groups wherein the cycloalkyl group is bridged to the acid moiety by an alkyl chain, preferably of from 1 to 3 carbon atoms, such as a methyl-cyclopropyl, methyl cyclopentyl or ethyl-cyclohexyl group. The cycloalkyl and alkylcycloalkyl groups may be optionally substituted by from 1 to 3 groups, including C₁-C₆ alkyl, C₁-C₆ alkyl, OH, halo, amino, nitro, cyano, etc., such as in a menthyl or alkyl-menthyl group. Also useful in the esters herein are cycloalkenyl or alkyl-cycloalkenyl groups wherein the carbocyclic ring has some amount of unsaturation, such as seen in a cyclohexenyl group.

Also useful are lower acyloxy-alkyl esters, such as acetoxymethyl, acetoxyethyl, aminocarbonyloxymethyl, pivaloyloxymethyl and pivaloyloxyethyl esters; lactonyl esters, such as phthalidyl and thiophthalidyl esters; lower alkoxyacyloxyalkyl esters, including methoxycarbonyloxymethyl, ethoxycarbonyloxyethyl and isopropoxycarbonyloxyethyl esters; alkoxyalkyl esters; choline esters; and alkylacylaminoalkyl esters, such as acetamidomethyl esters.

The invention also provides a pharmaceutical composition comprising a pharmaceutically or therapeutically effective amount of a compound of this invention and a pharmaceutically acceptable carrier.

Additionally, the invention provides a method of treating a proliferative disease in a patient in need thereof comprising administering a therapeutically effective amount of a compound of this invention.

The invention also provides the use of a compound of this invention for the manufacture of a medicament for the treatment of a proliferative disease.

Furthermore, the invention provides methods of treating cancer, restenosis, psoriasis, autoimmune disease, atherosclerosis, osteoarthritis, rheumatoid arthritis, heart failure, chronic pain, and neuropathic pain in a patient in need thereof comprising administering a therapeutically effective amount of a compound of this invention.

The invention also provides the use of a compound of this invention for the manufacture of a medicament for the treatment of cancer, restenosis, psoriasis, autoimmune disease, atherosclerosis, osteoarthritis, rheumatoid arthritis, heart failure, chronic pain, and neuropathic pain.

In addition, the invention provides a method for treating or inhibiting cancer in a patient in need thereof comprising administering a therapeutically effective amount of a compound of this invention in combination with radiation therapy, cryotherapy or at least one chemotherapeutic agent.

Certain terms are defined below and by their usage throughout this disclosure.

The terms “halogen” or “halo” in the present invention refer to a fluorine, bromine, chlorine, and iodine atom or fluoro, bromo, chloro, and iodo. The terms fluorine and fluoro, for example, are understood to be equivalent herein.

Alkyl groups, such as “C₁₋₆ alkyl”, include aliphatic chains (i.e., hydrocarbyl or hydrocarbon radical structures containing hydrogen and carbon atoms) with a free valence. Alkyl groups are understood to include straight chain and branched structures. Examples include methyl, ethyl, propyl, isopropyl, butyl, isobutyl, sec-butyl, t-butyl, pentyl, 2-pentyl, 3-pentyl, isopentyl, neopentyl, (R)-2-methylbutyl, (S)-2-methylbutyl, 3-methylbutyl, 2,3-dimethylpropyl, hexyl, and the like. The term “C₁₋₆ alkyl” includes within its definition the terms “C₁₋₄ alkyl” and “C₁₋₂ alkyl”.

Alkenyl groups are analogous to alkyl groups, but have at least one double bond (two adjacent sp² carbon atoms). Depending on the placement of a double bond and substituents, if any, the geometry of the double bond may be entgegen (E), or zusammen (Z), cis, or trans. Similarly, alkynyl groups have at least one triple bond (two adjacent sp carbon atoms). Unsaturated alkenyl or alkynyl groups may have one or more double or triple bonds, respectively, or a mixture thereof. Like alkyl groups, unsaturated groups may be straight chain or branched. Examples of alkenyls and alkynyls include vinyl, allyl, 2-methyl-2-propenyl, cis-2-butenyl, trans-2-butenyl, and acetyl.

Cycloalkyl groups, such as C₃₋₆ cycloalkyl, refer to a saturated hydrocarbon ring structure containing from 3 to 6 atoms. Typical C₃₋₆ cycloalkyl groups include cyclopropyl, cyclobutyl, cyclopentyl, cyclohexyl, and the like.

The present invention includes the hydrates and the pharmaceutically acceptable salts and solvates of the compounds of this invention. The compounds of this invention can possess a sufficiently basic functional group, and accordingly react with any of a number of inorganic and organic acids, to form a pharmaceutically acceptable salt.

The term “pharmaceutically acceptable salt” as used herein, refers to salts of the compounds of this invention which are substantially non-toxic to living organisms. Typical pharmaceutically acceptable salts include those salts prepared by reaction of the compounds of the present invention with a pharmaceutically acceptable mineral or organic acid. Such salts are also known as acid addition salts. Such salts include the pharmaceutically acceptable salts listed in Journal of Pharmaceutical Science, 1977;66:2-19, which are known to the skilled artisan.

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic, methanesulfonic acid, benzenesulfonic acid, oxalic acid, p-bromophenylsulfonic acid, carbonic acid, succinic acid, citric acid, benzoic acid, acetic acid, and the like. Example of such pharmaceutically acceptable salts are the sulfate, pyrosulfate, bisulfate, sulfite, bisulfite, phosphate, monohydrogenphosphate, dihydrogenphosphate, metaphosphate, pyrophosphate, bromide, hydrobromide, iodide, acetate, propionate, decanoate, caprate, caprylate, acrylate, ascorbate, formate, hydrochloride, dihydrochloride, isobutyrate, caproate, heptanoate, propiolate, glucuronate, glutamate, propionate, phenylpropionate, salicylate, oxalate, malonate, succinate, suberate, sebacate, fumarate, malate, maleate, hydroxymateate, mandelate, mesylate, nicotinate, isonicotinate, cinnamate, hippurate, nitrate, stearate, phthalate, teraphthalate, butyne-1,4-dioate, butyne-1,4-dicarboxylate, hexyne-1,4-dicarboxylate, hexyne-1,6-dioate, benzoate, chlorobenzoate, methylbenzoate, hydrozybenzoate, methoxybenzoate, dinitrobenzoate, o-acetoxybenzoate, naphthalene-2-benzoate, phthalate, p-toluenesulfonate, p-bromobenzenesulfonate, p-chlorobenzenesulfonate, xylenesulfonate, phenylacetate, trifluoroacetate, phenylpropionate, phenylbutyrate, citrate, lactate, α-hydroxybutyrate, glycolate, tartrate, hemi-tartrate, benzenesulfonate, methanesulfonate, ethanesulfonate, propanesulfonate, hydroxyethanesulfonate, 1-naphthalenesulfonate, 2-naphthalenesulfonate, 1,5-naphthalenedisulfonate, mandelate, tartarate, and the like. A preferred pharmaceutically acceptable salt is hydrochloride.

It should be recognized that the particular counterion forming a part of any salt of this inventions is usually not of a critical nature, so long as the salt as a whole is pharmacologically acceptable and as long as the counterion does not contribute undesired qualities to the salt as a whole. It is further understood that such salts may exist as a hydrate.

As used herein, the term “stereoisomer” refers to a compound made up of the same atoms bonded by the same bonds but having different three-dimensional structures which are not interchangeable. The three-dimensional structures are called configurations. As used herein, the term “enantiomer” refers to each of two stereoisomers whose molecules are nonsuperimposable mirror images of one another. The term “chiral center” refers to a carbon atom to which four different groups are attached. As used herein, the term “diastereomers” refers to stereoisomers which are not enantiomers. The terms “racemate” or “racemic mixture” refer to a mixture of enantiomers.

The enantiomers of compounds of the present invention can be resolved by one of ordinary skill in the art using standard techniques well-known in the art, such as those described by J. Jacques, et al., “Enantiomers, Racemates, and Resolutions”, John Wiley and Sons, Inc 1981. Examples of resolutions include recrystallization techniques or chiral chromatography.

Some of the compounds of the present invention have one or more chiral centers and may exist in a variety of stereoisomeric configurations. As a consequence of these chiral centers, the compounds of the present invention occur as racemates, mixtures of enantiomers and as individual enantiomers, as well as diastereomers and mixtures of diastereomers. All such racemates, enantiomers, and diastereomers are within the scope of the present invention.

The compounds of this invention can be prepared by techniques and procedures readily available to one of ordinary skill in the art, for example by following the procedures as set forth in the following Schemes, or analogous variants thereof. These synthetic strategies are further exemplified in examples below. These schemes are not intended to limit the scope of the invention in any way.

Cellular Assay for Measuring MEK Inhibition

MEK inhibitors were evaluated by determining their ability to inhibit phosphorylation of MAP kinase (ERK) in murine colon 26 (C26) carcinoma cells. Since ERK1 and ERK2 represent the only known substrates for MEK1 and MEK2, the measurement of inhibition of ERK phosphorylation in cells provides direct read out of cellular MEK inhibition by the compounds of the invention. Detection of phosphorylation of ERK was carried out either by Western blot or ELISA format. Briefly, the assays involve treatment of exponentially growing C26 cells with varying concentrations of the test compound (or vehicle control) for one hour at 37≡ C. For Western blot assay, cells were rinsed free of compound/vehicle and lysed in a solution containing 70 mM NaCl, 50 mM glycerol phosphate, 10 mM HEPES, HCl 7.4, 1% Triton X-100, 1 mM Na₃VO₄, 100 μM PMSF, 10 μM leupeptin and 10 μM pepstatin. Supernatants were then subjected to gel electrophoresis and hybridized to a primary antibody recognizing dually phosphorylated ERK1 and ERK2. To evaluate total MAPK levels, blots were subsequently ‘stripped’ and re-probed with a 1:1 mixture of polyclonal antibodies recognizing unphosphorylated ERK1 and ERK2. For pERK ELISA assay, pERK TiterZyme Enzyme immunometric Assay kits were acquired from Assay Designs, Inc (Ann Arbor, Mich.). Briefly, cells were harvested in lysis solution containing 50 mM β-glycerophosphate, 10 mM HEPES, pH7.4, 70 mM NaCl, 2 mM EDTA and 1% SDS and protein lysates were diluted 1:15 with supplied Assay buffer prior to the execution of the assay. The subsequent steps were carried out essentially as recommended by the manufacturer.

### DETAILED DESCRIPTION OF THE INVENTION

The present invention can be further understood by the following non-limiting examples.

### EXAMPLE 1

### 5-[3,4-Difluoro-2-(2-fluoro-4-iodo-phenylamino)-phenyl]-[1,3,4]oxadiazol-2-ylamine

m.p.=248-249° C.;

¹NMR (400 MHz; DMSO-d₆) 8.82 (1H, s), 7.63 (1H, dd, J=10.7 Hz, 1.9 Hz), 7.49-7.53 (1H, m), 7.43 (2H, s), 7.27-7.42 (1H, m), 7.20-7.25 (1H, m), 6.77-6.83 (1H, m).

MS(APCI+)=433

Anal. calcd/found for C₁₄H₈F₃IN₄O: C 38.91/39.26, H 1.87/2.02, N 12.96/12.67, F 13.19/12.92, I 29.37/29.64.

C26CPA1 IC₅₀=0.040 μM

### EXAMPLE 2

### 5-[5-Chloro-3,4-difluoro-2-(2-fluoro-4-iodo-phenylamino)-phenyl]-[1,3,4]oxadiazol-2-ylamine

m.p.=256-257° C.;

¹NMR (400 MHz; DMSO-d₆) 8.79 (1H, s), 7.60-7.65 (2H, m), 7.49 (2H, s), 7.40 (1H, d, J=9.5 Hz), 6.84-6.90 (1H, m)

Anal. calcd/found for C₁₄H₈F₃IN₄O: C 36.04/36.27, H 1.51/1.56, N 12.01/11.82, F 12.22/12.10, I 27.20/27.36

C26CPA1 IC₅₀=0.018 μM

### EXAMPLE 3

### 5-[2-(4-Bromo-2-fluoro-phenylamino)-3,4-difluoro-phenyl]-[1.3,4]oxadiazol-2-ylamine

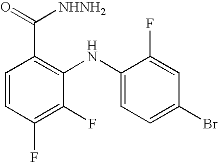

Step 1

To a stirring solution of 2-(4-bromo-2-fluoro-phenylamino)-3,4-difluoro-benzoic acid (1.0 g, 2.89 mmol) in DCM/THF (20 ml/20 ml), was added PyBOP (1.65 g, 3.17 mmol) and hydrazine (0.9 mL) and allowed to stir at room temperature overnight. The reaction mixture was then diluted with ethyl acetate, washed with saturated NaHCO₃, brine and dried over Na₂SO₄. Purification by column chromatography with hexane/ethyl acetate gave 2-(4-bromo-2-fluoro-phenylamino)-3,4-difluoro-benzoic acid hydrazide as a white solid (1.02 g, 98%).

Step 2

To a stirring solution of 2-(4-bromo-2-fluoro-phenylamino)-3,4-difluoro-benzoic acid hydrazide in 20 ml dioxane was added cyanogen bromide (0.338 g, 1.1 eq.) at room temperature, then NaHCO₃/water solution (270 mg/10 ml). The resulting mixture was stirred at room temperature overnight. The reaction mixture was concentrated and filtered and the afforded solid was washed with water. The solids were crystallized from hexane/ethyl acetate to afford 5-[2-(4-bromo-2-fluoro-phenylamino)-3,4-difluoro-phenyl]-[1,3,4]oxadiazol-2-ylamine as a white solid (0.7 g, 63%). m.p.=244.9-245.2° C.;

¹NMR (400 MHz; DMSO-d₆) 8.82 (1H, s), 7.54 (1H, dd, J=11.0 Hz, 2.2 Hz), 7.47-7.50 (1H, m), 7.42 (2H, s), 7.18-7.26 (2H, m), 6.92-6.97 (1H, m).

MS(APCI+)=385

Anal. calcd/found for C₁₄H₈F₃IN₄O: C 43.66/43.84, H 2.09/1.93, N 14.55/14.24, F 14.80/15.10, Br 20.75/20.75

C26CPA1 IC₅₀=0.140 μM

### EXAMPLE 4

### {5-[3,4-Difluoro-2-(2-fluoro-4-iodo-phenylamino)-phenyl]-[1,3,4]oxadiazol-2-yl}-ethyl-amine

m.p.=188-189° C.; ¹NMR (400 MHz; DMSO-d₆) 8.78 (s, 1H), 7.91 (t, 1H, J=11.5 Hz), 7.60 (dd, 1H, J=10.7, 1.7 Hz), 7.51-7.55 (m, 1H), 7.37-7.39 (m, 1H), 7.20-7.27 (m, 1H), 6.73-6.78 (m, 1H), 3.18-3.25 (m, 2H), 1.12 (t, 3H, J=22.5 Hz);

MS(APCI+)=461; Anal. calcd/found for C₁₆H₁₂F₃IN₄O: C 41.76/42.15, H 2.63/2.13, N 12.17/11.83

C26CPA1 IC₅₀=0.089 μM

### EXAMPLE 5

### Allyl-{5-[3,4-difluoro-2-(2-fluoro-4-iodo-phenylamino)-phenyl]-[1,3,4]oxadiazol-2-yl}-amine

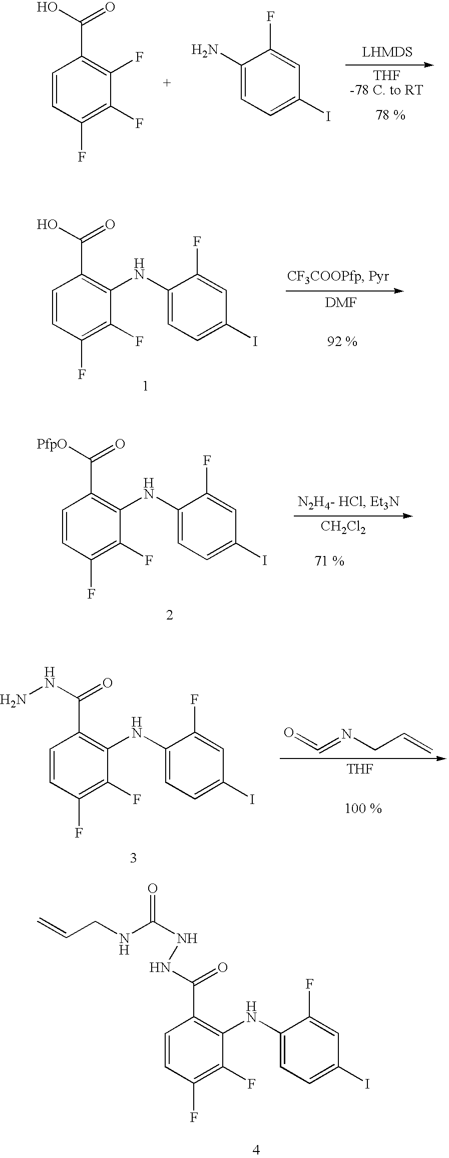

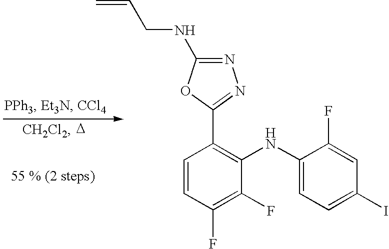

Step 1

To a stirred suspension of 2,3,4-trifluorobenzoic acid (78 g, 0.44 moles) in dry THF (1.25 L) under nitrogen at −78° C. was added LiHMDS (450 mL, 1 M solution in THF/hexanes) dropwise at such a rate that he temperature was maintained below −67° C. A dark orange solution was formed and this was stirred for another 20 minutes at −67° C. The mixture was designated as Solution A. To a stirred solution of 2-fluoro-4-iodoaniline (105 g, 0.44 moles, Aldrich) in dry THF (1.25 L) under nitrogen at −78° C. was added LiHMDS (450 mL, 1 M solution in THF/hexanes) dropwise at such a rate that he temperature was maintained below −67° C. The dark brown suspension was stirred for an additional 30 minutes at −67° C. The mixture was designated as Solution B. Solution A was transferred to solution B via a cannula under positive nitrogen pressure at −65° C. at such a rate to keep the temperature below −55° C. Then the mixture was slowly warmed to RT and stirred overnight. The reaction mixture was quenched with dry HCl in diethyl ether (1.5 L, freshly prepared, pH˜1-2. The solution was filtered through a layer of Celite. The filtrate was washed with aq. HCl (2M, 2×1L), brine and dried. Solvent was removed under reduced pressure to give a solid, which was suspended in hexanes-acetone (9:1, v/v, 150 mL) and stirred for 30 minutes. 3,4-difluoro-2-[(2-fluoro-4-iodophenyl)amino] benzoic acid was obtained by filtration as a white solid (135 g, 78%, mp. 195-197° C.).

Step 2

In an oven-dried three-neck, 2 L flask was taken 3,4-difluoro-2-[(2-fluoro-4-iodophenyl)amino] benzoic acid (196.7 g, 0.5 moles) and DMF (900 mL). To this stirred solution was added pyridine (44.4 mL, 43.5 g, 0.55 moles) at RT, and then pentafluorophenyl trifluoroacetate (95 mL, 154 g, 0.55 moles) was added dropwise within 30 minutes. The mixture was stirred at RT for 20 hours. The mixture was diluted with hexanes-diethyl ether (1:1, v/v, 3L) and washed successively with water (2×2L), 1M HCl (2×2L), saturated NaHCO₃ solution (2×2L) and finally with water (2×2L). The organic layer was dried and concentrated under reduced pressure to afford 2,3,4,5,6-pentafluorophenyl-3,4-difluoro-2-[(2-fluoro-4-iodophenyl)amino]benzoate as a red oil (92.3%, 258.5 g).

Step 3

To a stirred solution of anhydrous hydrazine (28.61 g, 0.89 moles in DCM (2L) was added a solution of 2,3,4,5,6-pentafluorophenyl-3,4-difluoro-2-[(2-fluoro-4-iodophenyl)amino]benzoate (250 g, 0.447 moles) in DCM (800 mL) dropwise at 0° C. The mixture was allowed to warm to RT and stirred for 3 hours. The precipitated white solid was collected by filtration, an the filtrate was concentrated to dryness. The solid and the residue were combined and taken into ethyl acetate (4L), washed with water (2×3L), brine (2×3L), dried and concentrated under reduced pressure to approximately 1.5 L. The precipitated solid was re-dissolved by heating the mixture to boiling temperature. Hexane (1L) was added and the solution was kept at RT overnight. N-amino{3,4-difluoro-2-[(2-fluoro-4-iodopheyl)amino]phenyl} carboxamide was obtained as colorless needles (109.5 g) as crop I. The mother liquor was concentrated to 1L to give a second crop as colorless needles (20.2 g). Total 129.7 g in 71.2% yield, mp. 168-169° C.

Step 4

To a stirring solution of N-amino{3,4-difluoro-2-[(2-fluoro-4-iodopheyl)amino]phenyl} carboxamide (0.250 g, 0.614 mmol) in THF (10 mL) was added allylisocyanate (0.060 mL, 0.676 mmol) and allowed to stir at ambient temperature for 2 hours. The mixture was concentrated under reduced pressure which afforded 4′-allyl-1′[3,4-difluoro-2-(2-fluoro-4-iodo-phenylamino)]-semicarbazole as a foam/solid (0.343 g, >100% due to remaining THF).

Step 5

To a stirring solution of 4′-allyl-1′[3,4-difluoro-2-(2-fluoro-4-iodo-phenylamino)]-semicarbazole (0.301 g, 0.614 mmol) in dichloromethane (15 mL) was added triphenylphosphine (0.241 g, 0.952 mmol), triethylamine (0.13 mL, 0.921 mmol) and carbon tetrachloride (0.20 mL, 2.46 mmol) and the mixture was heated to reflux (oil bath was set at 46° C.). After one hour of refluxing, triphenylphosphine (0.241 g, 0.952 mmol), triethylamine (0.13 mL, 0.921 mmol) and carbon tetrachloride,(0.20 mL, 2.46 mmol) were added and allowed to reflux an additional 3 hours. The reaction mixture was allowed to cool then partitioned between dichloromethane and water. Organics were washed twice with water, then collected and dried over Na₂SO₄, filtered and concentrated in vacuo. Silica column purification was performed with 2:1 hexane/ethyl acetate and afforded allyl-{5-[3,4-difluoro-2-(2-fluoro-4-iodo-phenylamino)-phenyl]-[1,3,4]oxadiazol-2-yl}-amine (0.160 g, 55%). m.p.=175-178° C.; ¹NMR (400 MHz; DMSO-d₆) 8.76 (s, 1H), 8.14 (t, 1H, J=5.9 Hz), 7.60 (dd, 1H, J=10.7, 1.7 Hz), 7.51-7.54 (m, 1H), 7.37 (d, 1H, J=8.5 Hz), 7.20-7.27 (m, 1H), 6.72-6.78 (m, 1H), 5.80-5.90 (m, 1H), 5.17-5.21 (m, 1H), 5.04-5.09 (m, 1H), 3.80-3.82 (m, 2H); MS(APCI+)=473; Anal. calcd/found, for C₁₇H₁₂F₃IN₄O: C 43.24/43.66, H 2.56/2.55, N 11.86/11.72, F 12.07/11.97. C26CPA1 IC₅₀=0.009 μM

### EXAMPLE 6

### {5-[3,4-Difluoro-2-(2-fluoro-4-iodo-phenylamino)-phenyl]-[1,3,4]oxadiazol-2-yl}-(2.4-dimethoxy-phenyl)-amine

m.p.=185-186° C.; ¹NMR (400 MHz; DMSO-d₆) 9.60 (s, 1H), 8.75 (s, 1H), 7.59-7.63 (m, 2H), 7.51-7.54 (m, 1H), 7.38 (d, 1H, J=8.3 Hz), 7.23-7.29 (m, 1H), 6.74-6.80 (m, 1H), 6.61 (d, 1H, J=2.4 Hz), 6.48 (dd, 1H, J=8.8, 2.7 Hz), 3.77 (s, 3H), 3.72 (s, 3H); MS(APCI+)=569; Anal. calcd/found for C₂₂H₁₆F₃IN₄O₃ with 0.19 moles of residual C₄H₈O₂: C 46.73/47.09, H 3.02/3.23, N 9.58/9.19, F 9.74/9.70 C26CPA1 IC₅₀=>1.0 μM

### EXAMPLE 7

### N-{5-[3,4-Difluoro-2-(2-fluoro-4-iodo-phenylamino)-phenyl]-[1,3,4]oxadiazol-2-yl}-N′,N′-dimethyl-benzene-1,4-diamine

m.p.=230-233° C.; ¹NMR (400 MHz; DMSO-d₆) 10.31 (s, 1H), 8.79 (s, 1H), 7.56-7.64 (m, 2H), 7.25-7.41 (m, 4H), 6.76-6.81 (m, 1H), 6.69 (d, 2H, J=9.0 Hz), 2.82 (s, 6H); MS(APCI+)=552; Anal. calcd/found for C₂₂H₁₇F₃IN₅O: C 47.93/48.11, H 3.11/2.93, N 12.70/12.59 F 10.34/10.42.

C26CPA1 IC₅₀=>1.0 μM

### EXAMPLE 8

### 3-{5-[3,4-Difluoro-2-(2-fluoro-4-iodo-phenylamino)-phenyl]-[1,3,4]oxadiazol-2-ylamino}-propane-1,2-diol

To a stirring suspension of allyl-{5-[3,4-difluoro-2-(2-fluoro-4-iodo-phenylamino)-phenyl]-[1,3,4]oxadiazol-2-yl}-amine (0.11 g, 0.233 mmol) in tert-butanol (4 mL) at 45° C. is added N-methyl morpholine-N-oxide (0.030 g, 0.256 mmol), a catalytic amount of potassium osmate dihydrate and water (0.4 mL) and allowed to stir at 45° C. for 18 hours. A second amount of potassium osmate dihydrate was added and allowed to stir an additional 3 hours. Saturated sodium metabisulfate solution was added (20 mL) and allowed to stir for 30 minutes. The reaction mixture was filtered and the filtrate was extracted with ethyl acetate. Organics were washed twice with water and twice with brine. Organics were collected and dried over Na₂SO₄, filtered and concentrated in vacuo. Silica column purification was performed with 1% methanol in dichloromethane to 5% methanol in dichloromethane and afforded the title compound as a white solid (0.047 g, 47%). m.p.=157-159° C.; ¹NMR (400 MHz; DMSO-d₆) 8.80 (s, 1H), 7.91 (t, 1H, J=5.9 Hz), 7.62 (dd, 1H, J=10.8, 1.7 Hz), 7.51-7.54 (m, 1H), 7.39 (d, 1H, J=8.5 Hz), 7.21-7.27 (m, 1H), 6.75-6.80 (m, 1H), 4.83 (d, 1H, J=5.1 Hz), 4.59 (t, 1H, J=5.9 Hz), 3.60-3.63 (m, 1H), 3.3 (3H under HDO), 3.07-3.14 (m, 1H); MS(APCI+)=507; Anal. calcd/found for C₁₇H₁₄F₃IN₄O₃: C 40.34/40.07, H 2.79/2.87, N 11.07/10.06. C26CPA1 IC₅₀=0.067 μM

### EXAMPLE 9

### {5-[3,4-Difluoro-2-(2-fluoro-4-iodo-phenylamino)-phenyl]-[1,3,4]oxadiazol-2-yl}-(3-morpholin-4-yl-propyl)-amine Hydrochloride Salt

m.p.=134.136° C.; ¹NMR (400 MHz; DMSO-d₆) 8.79 (s, 1H), 8.09-8.10 (m, 1H), 7.61-7.64 (m, 1H), 7.52-7.54 (m, 1H), 7.39 (d, 1H, J=8.3 Hz), 7.22-7.29 (m, 1H), 6.74-6.77 (m, 1H), 3.92 (d, 2H, J=11.7 Hz), 3.67 (t, 2H, J=12.0 Hz), 3.28-3.38 (m, 4H), 2.98-3.11 (m, 4H), 1.96 (m, 2H); MS(APCI+)=560; Anal. calcd/found for C₂₁H₂₁F₃IN₅O2 with 2.41 moles of HCl: C 38.97/38.59, H 3.65/3.78, N 10.82/10.57. C26CPA1 IC₅₀=0.311 μM

### EXAMPLE 10

### N-{5-[3,4-Difluoro-2-(2-fluoro-4-iodo-phenylamino)-phenyl]-[1,3,4]oxadiazol-2-yl}-N′,N′-diethyl-propane-1,3-diamine

Step 5

To a stirring solution of N,N-diethylpropylamine (0.038 mL, 0.270 mmol) in anhydrous DMF (1 mL) is added carbonyldiimidazole (0.044 g, 0.270 mmol) and allowed to stir at ambient temperature. After 3 hours, 2-(4-iodo-2-fluoro-phenylamino)-3,4-difluoro-benzoic acid hydrazide (0.1 g, 0.270 mmol) was added and heated to 75° C. After stirring for an additional 17 hours, the reaction mixture was partitioned between ethyl acetate and water. Organics were washed twice with water and twice with saturated NaHCO₃. Organics were collected, dried over Na₂SO₄, filtered and concentrated in vacuo. Afforded 4′-N-N-diethylpropylamine-1′-[3,4-difluoro-2-(2-fluoro-4-iodo-phenylamino)]-semicarbazole as a light yellow solid (0.118 g, 85.5%).

Step 5

To a stirring solution of 4′-N-N-diethylpropylamine-1′-[3,4-difluoro-2-(2fluoro-4-iodo-phenylamino)]-semicarbazole (0.118 g, 0.21 mmol) in dichloromethane (4 mL) was added triphenylphosphine (0.084 g, 0.32 mmol), triethylamine (0.12 mL, 0.84 mmol) and carbon tetrachloride (0.026 mL, 0.32 mmol) and heated to 45° C. for 1 hour. A second portion of triphenylphosphine (0.084 g, 0.32 mmol), triethylamine (0.12 mL, 0.84 mmol) and carbon tetrachloride (0.026 mL, 0.32 mmol) was added and the mixture was allowed to reflux an additional three hours. The reaction mixture was allowed to cool to ambient temperature and stir overnight. The mixture was partitioned between dichloromethane and water. Organics were washed twice with water, twice with saturated NaCl. Organics were collected, dried over Na₂SO₄, filtered and concentrated in vacuo. Silica column chromatography was performed in 9:1 dichloromethane/acetone to 1:1 dichloromethane/acetone with 0.5% triethylamine.

A further crystallization from hexanes afforded N-{5-[3,4-Difluoro-2-(2-fluoro-4-iodo-phenylamino)-phenyl]-[1,3,4]oxadiazol-2-yl}-N′,N′-diethyl-propane-1,3-diamine (0.068 g, 59.6%). m.p.=116-118° C.; ¹NMR (400 MHz; DMSO-d₆) 8.78 (s, 1H), 7.94 (t, 1H, J=5.4 Hz), 7.61 (d, 1H, J=10.7 Hz), 7.51-7.55 (m, 1H), 7.38 (d, 1H, J=8.5 Hz), 7.21-7.28 (m, 1H), 3.18-3.23 (m, 2H), 2.36-2.47 (m, 6H), 1.62 (quint, 2H, J=6.8 Hz), 0.90 (t, 6H, J=7.1 Hz); MS(APCI+)=546.5; Anal. calcd/found for C₂₁H₂₃F₃IN₅O: C 46.25/46.06, H 4.25/4.14, N 12.84/12.61. C26CPA1 IC₅₀=0.153 μM

### EXAMPLE 11

### N′1′-{5-[3,4-Difluoro-2-(2-fluoro-4-iodo-phenylamino)-phenyl]-[1,3,4]oxadiazol-2-yl}-ethane-1,2-diamine as a Trifluoro-acetic Acid Salt

The title compound was prepared using steps 5 and 6 as seen in Example 18, below.

Step 7

To a stirring solution of the product from step 6 (0.031 g, 0.054 mmol) in dichloromethane (5 mL) was added trifluoroacetic acid (0.5 mL) and allowed to stir at ambient temperature for 30 minutes. The reaction mixture was then concentrated in vacuo to afford a yellow oil. Diethyl ether was added, which afforded the title compound as a white solid. m.p.=156-158° C.; ¹NMR (400 MHz; DMSO-d₆) 8.79 (s, 1H), 8.12 (m, 1H), 7.61-7.64 (m, 3H), 7.63 (d, 1H, J=10.7 Hz), 7.53-7.57 (m, 1H), 7.40 (d, 1H, J=8.5 Hz), 7.26 (dd, 1H, J=16.9, 9.0 Hz), 6.72-6.81 (m, 1H), 3.43-3.45 (m, 2H), 3.01 (t, 2H, J=5.9 Hz); MS(APCI+)=476; Anal. calcd/found for C₁₆H₁₃F₃IN₅O with 1.00 moles of C₂HF₃O₂ and 0.20 moles of residual C₄H₁₀O₁: C 37.38/37.36, H 2.67/2.60, N 11.59/11.25, F 18.87/18.54. C26CPA1 IC₅₀=0.015 μM

### EXAMPLE 12

### N-{5-[3,4-Difluoro-2-(2-fluoro-4-iodo-phenylamino)-phenyl]-[1,3,4]oxadiazol-2-yl}-N,N′,N′-trimethyl-propane-1,3-diamine as a Hydrochloride Salt

m.p.=123-126° C.; ¹NMR (400 MHz; CD₃OD) 7.66-7.70 (m, 1H), 7.48 (dd, 1H, J=10.4, 1.6 Hz), 7.38 (d, 1H, J=8.5 Hz), 7.05-7.12 (m, 1H), 6.66-6.72 (m, 1H), 3.57 (t, 2H, J=6.8 Hz), 3.15-3.19 (m, 5H), 2.89 (s, 6H), 2.05-2.12 (m, 2H); MS(APCI+)=532.5; Anal. calcd/found for C₂₀H₂₁F₃IN₅O with 1.80 moles of HCl and 0.05 moles of residual C₄H₈O₂: C 40.35/40.56, H 3.89/4.03, N 11.65/11.25. C26CPA1 IC₅₀=>1.0 μM

### EXAMPLE 13

### {5-[3,4-Difluoro-2-(2-fluoro-4-iodo-phenylamino)-phenyl]-[1,3,4]oxadiazol-2-yl}-(2-piperidin-1-yl-ethyl)-amine as a Hydrochloride Salt

m.p.=191-193° C0.; ¹NMR (400 MHz; CD₃OD) 7.61-7.65 (m, 1H), 7.48 (dd, 1H, J=10.7, 2.0 Hz), 7.39 (dd, 1H, J=8.5, 1.2), 7.04-7.11 (m, 1H), 6.69-6.74 (m, 1H), 3.76 (t, 2H, J=6.4 Hz), 3.62 (d, 2H, J=11.7 Hz), 3.38 (t, 2H, J=6.1 Hz), 2.96-3.03 (m, 2H), 1.93-1.97 (m, 2H), 1.72-1.85 (m, 3H), 1.50-1.56 (m, 1H); MS(APCI+)=544; Anal. calcd/found for C₂₁H₂₁F₃IN₅O with 2.10 moles of HCl: C 40.69/40.30,H 3.76/3.80, N 11.30/11.11. C26CPA1 IC₅₀=0.382 μM

### EXAMPLE 14

### N′1′-{5-[3,4-Difluoro-2-(2-fluoro-4-iodo-phenylamino)-phenyl]-[1,3,4]oxadiazol-2-yl}-butane-1,4-diamine as a Hydrochloride Salt

m.p.=157-159° C.; ¹NMR (400 MHz; DMSO-d₆) 8.79 (s, 1H), 8.04 (t, 1H, J=5.6 Hz), 7.73 (bs, 2H), 7.62 (dd, 1H, J=10.7, 2.0 Hz), 7.51-7.54 (m, 1H), 7.39 (d, 1H, J=8.3 Hz), 7.22-7.28 (m, 1H), 6.73-6.79 (m, 1H), 3.20-3.22 (m, 2H), 2.75-2.77 (m, 2H), 1.56-1.57 (m, 4H); MS(APCI+)=504; Anal. calcd/found for C₁₈H₁₇F₃IN₅O with 1.76 moles of HCl: C 38.10/38.13, H 3.33/3.52, N 12.34/11.95, F 10.04/10.38. C26CPA1 IC₅₀=0.045 μM

### EXAMPLE 15

### N′1′-{5-[3,4-Difluoro-2-(2-fluoro-4-iodo-phenylamino)-phenyl]-[1,3,4]oxadiazol-2-yl}-propane-1,3-diamine as a Hydrochloride Salt

m.p.=173-176° C.; ¹NMR (400 MHz; DMSO-d₆) 8.79 (s, 1H), 8.60 (t, 1H, J=5.9 Hz), 7.74 (bs, 2H), 7.62 (dd, 1H, J=10.7, 1.7 Hz), 7.52-7.55 (m, 1H), 7.39 (d, 1H, J=8.5 Hz), 722-7.29 (m, 1H), 6.74-6.80 (m, 1H), 3.25-3.30 (m, 2H), 2.82-2.85 (m, 2H), 1.80-1.86 (m, 2H); MS(APCI+)=490; Anal. calcd/found for C₁₇H₁₅F₃IN₅O*HCl with 0.10 moles residual C₄H₁₀O: C 37.53/37.25, H 3.20/3.57, N 12.58/12.29 F 10.24/10.16. C26CPA1 IC₅₀=0.076 μM

### EXAMPLE 16

### N-{5-[3,4-Difluoro-2-(2-fluoro-4-iodo-phenylamino)-phenyl]-[1,3,4]oxadiazol-2-yl}-N′,N′-dimethyl-propane-1,3-diamine as a Hydrochloride Salt

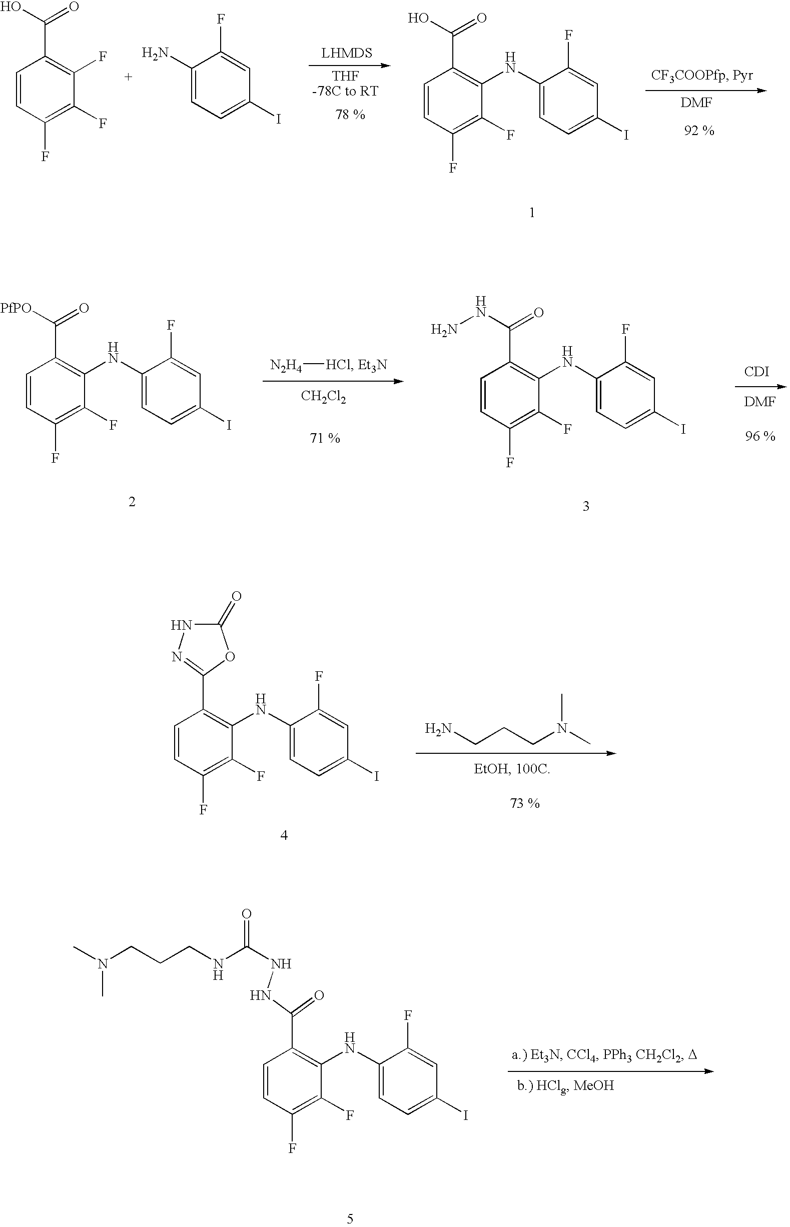

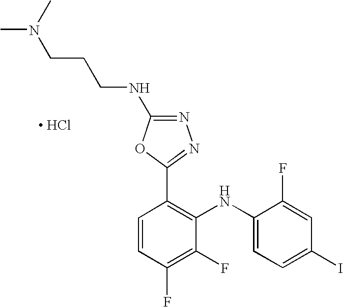

Step 1

To a stirred suspension of 2,3,4-trifluorobenzoic acid (78 g, 0.44 moles) in dry THF (1.25 L) under nitrogen at −78° C. was added LiHMDS (450 mL, 1 M solution in THF/hexanes) dropwise at such a rate that he temperature was maintained below −67° C. A dark orange solution was formed and this was stirred for another 20 minutes at −67° C. The mixture was designated as Solution A. To a stirred solution of 2-fluoro-4-iodoaniline (105 g, 0.44 moles, Aldrich) in dry THF (1.25 L) under nitrogen at −78° C. was added LiHMDS (450 mL, 1 M solution in THF/hexanes) dropwise at such a rate that he temperature was maintained below −67° C. The dark brown suspension was stirred for an additional 30 minutes at −67° C. The mixture was designated as Solution B. Solution A was transferred to solution B via a cannula under positive nitrogen pressure at −65° C. at such a rate to keep the temperature below −55° C. Then the mixture was slowly warmed to RT and stirred overnight. The reaction mixture was quenched with dry HCl in diethyl ether (1.5 L, freshly prepared, pH˜1-2. The solution was filtered through a layer of Celite. The filtrate was washed with aq. HCl (2M, 2×1L), brine and dried. Solvent was removed under reduced pressure to give a solid, which was suspended in hexanes-acetone (9:1, v/v, 150 mL) and stirred for 30 minutes. 3,4-difluoro-2-[(2-fluoro-4-iodophenyl)amino] benzoic acid was obtained by filtration as a white solid (135 g, 78%, mp. 195-197° C.).

Step 2

In an oven-dried three-neck, 2 L flask was taken 3,4-difluoro-2-[(2-fluoro-4-iodophenyl)amino] benzoic acid (196.7 g, 0.5 moles) and DMF (900 mL). To this stirred solution was added pyridine (44.4 mL, 43.5 g, 0.55 moles) at RT, and then pentafluorophenyl trifluoroacetate (95 mL, 154 g, 0.55 moles) was added dropwise within 30 minutes. The mixture was stirred at RT for 20 hours. The mixture was diluted with hexanes-diethyl ether (1:1, v/v, 3L) and washed successively with water (2×2L), 1M HCl (2×2L), saturated NaHCO₃ solution (2×2L) and finally with water (2×2L). The organic layer was dried and concentrated under reduced pressure to afford 2,3,4,5,6-pentafluorophenyl-3,4-difluoro-2-[(2-fluoro-4-iodophenyl)amino]benzoate as a red oil (92.3%, 258.5 g).

Step 3

To a stirred solution of anhydrous hydrazine (28.61 g, 0.89 moles in DCM (2L) was added a solution of 2,3,4,5,6-pentafluorophenyl-3,4-difluoro-2-[(2-fluoro-4-iodophenyl)amino]benzoate (250 g, 0.447 moles) in DCM (800 mL) dropwise at 0° C. The mixture was allowed to warm to RT and stirred for 3 hours. The precipitated white solid was collected by filtration, an the filtrate was concentrated to dryness. The solid and the residue were combined and taken into ethyl acetate (4L), washed with water (2×3L), brine (2×3L), dried and concentrated under reduced pressure to approximately 1.5 L. The precipitated solid was re-dissolved by heating the mixture to boiling temperature. Hexane (1L) was added and the solution was kept at RT overnight. N-amino{3,4-difluoro-2-[(2-fluoro-4-iodopheyl)amino]phenyl} carboxamide was obtained as colorless needles (109.5 g) as crop I. The mother liquor was concentrated to 1L to give a second crop as colorless needles (20.2 g). Total 129.7 g in 71.2% yield, mp. 168-169° C.

Step 4

To a solution of N-amino {3,4-difluoro-2-[(2-fluoro-4-iodopheyl)amino]phenyl} carboxamide (50 g, 123 mmoles) in DMF (250 mL) was added 1,1′-carbonyldiimidazole (20.91 g, 129 mmoles, 1.05 eq). The mixture was stirred at RT for 5 hours, then poured into ethyl acetate (2.5L) and washed with water (2×2.5L), brine (2×2.5 L), dried and evaporated to give 5-[3,4-difluoro-2-(2-fluoro-4-iodo-phenylamino)-phenyl]-3H-[1,3,4]oxadiazol-2-one as a white solid (53.2 g). Further crystallization from THF-hexanes gave the pure product as colorless needles in 96% yield, 51 g, mp. 224-225° C.

Step 5

To a stirring suspension of 5-[3,4-difluoro-2-(2-fluoro-4-iodo-phenylamino)-phenyl]-3H-[1,3,4]oxadiazol-2-one (0.133 g, 0.307 mmol) in ethanol (5 mL) was added N,N-dimethyl-propane-1,3-diamine (0.043 mL, 0.338 mmol) and heated to reflux (oil bath temperature was set to 100° C.). After reaction refluxed for 19 hours another mole equivalence of N,N-dimethyl-propane-1,3-diamine (0.038 mL, 0.307 mmol) was added and heated to reflux an additional 4 hours. The mixture was allowed to cool and concentrated in vacuo. The afforded residue was dissolved in ethyl acetate and partitioned with water. Organics were washed twice with brine then collected and dried over Na₂SO₄, filtered and concentrated in vacuo. Afforded 4′-N-ethylcarbamic acid tert-butyl ester-1′-[3,4-difluoro-2-(2-fluoro-4-iodo-phenylamino)]-semicarbazole as a brown foam/solid (0.120 g, 73.2%).

Step 6

Part A

To a stirring solution of 4′-N-ethylcarbamic acid tert-butyl ester-1′-[3,4-difluoro-2-(2-fluoro-4-iodo-phenylamino)]-semicarbazole (0.097 g, 0.186 mmol) in dichloromethane (2 mL) is added triphenylphosphine (0.073 g, 0.279 mmol), triethylamine (0.1 mL, 0.744 mmol) and carbon tetrachloride (0.022 mL, 0.279 mmol) and the mixture was heated to reflux (oil bath was set at 46° C.). After one hour of refluxing, triphenylphosphine (0.073 g, 0.279 mmol), triethylamine (0.1 mL, 0.744 mmol) and carbon tetrachloride (0.022 mL, 0.279 mmol) were added and allowed to reflux an additional 3 hours. The reaction mixture was allowed to cool then partitioned between dichloromethane and water. Organics were washed twice with water, then collected and dried over Na₂SO₄, filtered and concentrated in vacuo. Silica column purification was performed with 1:1 dichloromethane/acetone to the afforded yellow foam (1.12 g) was added 3:1 hexanes/ethyl acetate which afforded a yellow foam/solid (0.05 g, 43.5%).

Part B

To a stirring suspension of the product from part A (0.050 g, 1.103 mmol) in methanol (3 mL) is bubbled in HCl gas for approximately 1 minute. The mixture was allowed to stir at ambient temperature for 15 minutes. The reaction mixture was concentrated in vacuo to afford a yellow oil. Diethyl ether was added and mixture was allowed to stand overnight. The afforded white solids were tritrated several times with diethyl ether and dried in vacuo at 50° C. Afforded the title compound as a hydrochloride salt (1.75 mole equivalence of HCl and 0.30 mole equivalence of H₂0). m.p.=168-170° C.; ¹NMR (400 MHz; DMSO-d₆) 8079 (s, 1H), 8.07 (m, 1H), 7.62 (dd, 1H, J=10.7, 2.0), 7.52-7.55 (m, 1H), 7.39 (d, 1H, J=8.5), 7.22-7.29 (m, 1H), 6.74-6.80 (m, 1H), 3.27 (q, 2H, J=6.8 Hz), 3.04-3.08 (m, 2H), 2.72 (s, 3H), 2.73 (s, 3H), 1.90 (m, 2H); MS(APCI+)=518; Anal. calcd/found for C₁₉H₁₉F₃IN₅O* (1.75 eq HCl and 0.30 eq H₂O): C 38.91/38.56, H 3.67/3.72, N 11.94/11.56, Cl 10.58/10.22. C26CPA1 IC₅₀=0.136 μM

### EXAMPLE 17

### N-{5-[3,4-Difluoro-2-(2-fluoro-4-iodo-phenylamino)-phenyl]-[1,3,4]oxadiazol-2-yl}-N′,N′-dimethyl-ethane-1,2-diamine as a Hydrochloride Salt

m.p.=184-187° C.; ¹NMR (400 MHz; CD₃OD) 7.59-7.63 (m, 1H), 7.48 (dd, 1H, J=10.4, 2.0 Hz), 7.39 (d, 1H, J=8.3 Hz), 7.03-7.09 (m, 1H), 6.69-6.74 (m, 1H), 3.68 (m, 2H), 3.3 (2H under CD₃OD), 2.75-2.90 (cm, 6H); MS(APCI+)=504; HPLC=7.322 min at 254 nm. C26CPA1 IC₅₀=0.140 μM

### EXAMPLE 18

### N-{5-[3,4-Difluoro-2-(2-fluoro-4-iodo-phenylamino)-phenyl]-[1,3,4]oxadiazol-2-yl}-N′-methyl-ethane-1,2-diamine as a Hydrochloride Salt

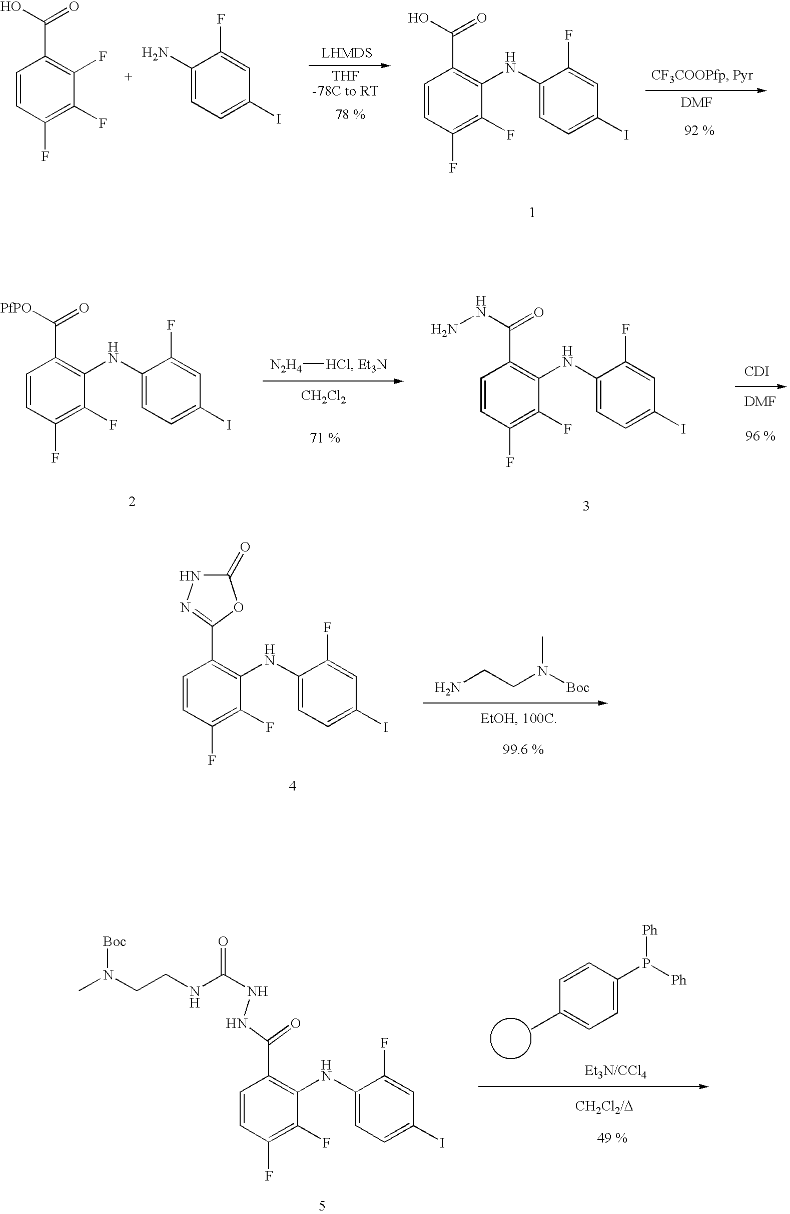

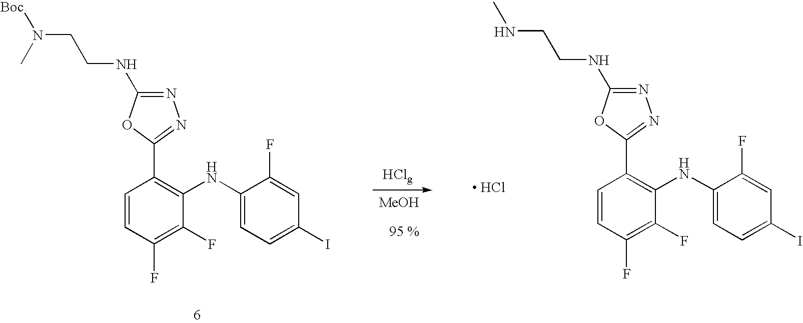

Step 1

To a stirred suspension of 2,3,4-trifluorobenzoic acid (78 g, 0.44 moles) in dry THF (1.25 L) under nitrogen at −78° C. was added LiHMDS (450 mL, 1 M solution in THF/hexanes) dropwise at such a rate that he temperature was maintained below −67° C. A dark orange solution was formed and this was stirred for another 20 minutes at −67° C. The mixture was designated as Solution A. To a stirred solution of 2-fluoro-4-iodoaniline (105 g, 0.44 moles, Aldrich) in dry THF (1.25 L) under nitrogen at −78° C. was added LiHMDS (450 mL, 1 M solution in THF/hexanes) dropwise at such a rate that he temperature was maintained below −67° C. The dark brown suspension was stirred for an additional 30 minutes at −67° C. The mixture was designated as Solution B. Solution A was transferred to solution B via a cannula under positive nitrogen pressure at −65° C. at such a rate to keep the temperature below −55° C. Then the mixture was slowly warmed to RT and stirred overnight. The reaction mixture was quenched with dry HCl in diethyl ether (1.5 L, freshly prepared, pH˜1-2. The solution was filtered through a layer of Celite. The filtrate was washed with aq. HCl (2M, 2×1L), brine and dried. Solvent was removed under reduced pressure to give a solid, which was suspended in hexanes-acetone (9:1, v/v, 150 mL) and stirred for 30 minutes. 3,4-difluoro-2-[(2-fluoro-4-iodophenyl)amino] benzoic acid was obtained by filtration as a white solid (135 g, 78%, mp. 195-197° C.).

Step 2

In an oven-dried three-neck, 2 L flask was taken 3,4-difluoro-2-[(2-fluoro-4-iodophenyl)amino] benzoic acid (196.7 g, 0.5 moles) and DMF (900 mL). To this stirred solution was added pyridine (44.4 mL, 43.5 g, 0.55 moles) at RT, and then pentafluorophenyl trifluoroacetate (95 mL, 154 g, 0.55 moles) was added dropwise within 30 minutes. The mixture was stirred at RT for 20 hours. The mixture was diluted with hexanes-diethyl ether (1:1, v/v, 3L) and washed successively with water (2×2L), 1M HCl (2×2L), saturated NaHCO₃ solution (2×2L) and finally with water (2×2L). The organic layer was dried and concentrated under reduced pressure to afford 2,3,4,5,6-pentafluorophenyl-3,4-difluoro-2-[(2-fluoro-4-iodophenyl)amino]benzoate as a red oil (92.3%, 258.5 g).

Step 3

To a stirred solution of anhydrous hydrazine (28.61 g, 0.89 moles in DCM (2L) was added a solution of 2,3,4,5,6-pentafluorophenyl-3,4-difluoro-2-[(2-fluoro-4-iodophenyl)amino]benzoate (250 g, 0.447 moles) in DCM (800 mL) dropwise at 0° C. The mixture was allowed to warn to RT and stirred for 3 hours. The precipitated white solid was collected by filtration, an the filtrate was concentrated to dryness. The solid and the residue were combined and taken into ethyl acetate (4L), washed with water (2×3L), brine (2×3L), dried and concentrated under reduced pressure to approximately 1.5 L. The precipitated solid was re-dissolved by heating the mixture to boiling temperature. Hexane (1L) was added and the solution was kept at RT overnight. N-amino{3,4-difluoro-2-[(2-fluoro-4-iodopheyl)amino]phenyl} carboxamide was obtained as colorless needles (109.5 g) as crop I. The mother liquor was concentrated to 1L to give a second crop as colorless needles (20.2 g). Total 129.7 g in 71.2% yield, mp. 168-169° C.

Step 4

To a solution of N-amino {3,4-difluoro-2-[(2-fluoro-4-iodopheyl)amino]phenyl} carboxamide (50 g, 123 mmoles) in DMF (250 mL) was added 1,1′-carbonyldiimidazole (20.91 g, 129 mmoles, 1.05 eq). The mixture was stirred at RT for 5 hours, then poured into ethyl acetate (2.5L) and washed with water (2×2.5L), brine (2×2.5 L), dried and evaporated to give 5-[3,4-difluoro-2-(2-fluoro-4-iodo-phenylamino)-phenyl]-3H-[1,3,4]oxadiazol-2-one as a white solid (53.2 g). Further crystallization from THF-hexanes gave the pure product as colorless needles in 96% yield, 51 g, mp. 224-225° C.

Step 5

To a stirring suspension of 5-[3,4-difluoro-2-(2-fluoro-4-iodo-phenylamino)-phenyl]-3H-[1,3,4]oxadiazol-2-one (1.017 g, 2.35 mmol) in ethanol (25 mL, 0.094 M) was added N-Boc-N-methylethylenediamine (Fluka, 0.84 mL, 4.70 mmol) and heated to reflux (oil bath temperature was set to 100° C.). After reaction refluxed for 16 hours, the mixture was allowed to cool and concentrated in vacuo. The afforded residue was dissolved in ethyl acetate and partitioned with sat. NaHCO₃. Organics were washed twice with brine then collected and dried over Na₂SO₄, filtered and concentrated in vacuo. Afforded of 4′-(2-amino-ethyl)-methyl-carbamic acid tert-butyl ester-1′-[3,4-difluoro-2-(2-fluoro-4-iodo-phenylamino)]-semicarbazole as a cream foam/solid (1.420 g, 99.6%).

Step 6

To a stirring solution of 4′-(2-amino-ethyl)-methyl-carbamic acid tert-butyl ester-1′-[3,4-difluoro-2-(2-fluoro-4-iodo-phenylamino)]-semicarbazole (1.37 g, 2.256 mmol) in dichloromethane (40 mL, 0.05M) is added resin bound triphenylphosphine# (3.9 g, 6.768 mmol)**, triethylamine (2.5 mL, 18.05 mmol) and carbon tetrachloride (0.54 mL, 6.768 mmol) and the mixture was heated to reflux (oil bath was set at 46° C.). Reaction was complete after 3 hours. The reaction mixture was filtered and the resin was rinsed with 50 mL of 5% methanol in dichloromethane. The filtrate was collected and concentrated in vacuo. To the afforded yellow foam (1.12 g) was added 3:1 hexanes/ethyl acetate which afforded (2-{5-[3,4-Difluoro-2-(2-fluoro-4-iodo-phenylamino)-phenyl]-[1,3,4]oxadiazol-2-ylamino}-ethyl)-methyl-carbamic acid tert-butyl ester (0.65 g, 48.9) as a white solid. # triphenylphosine can also be used, but will then require column chromatography. **2.0 mole equivalence of PS-PPh₃ is sufficient for reaction completion.

Step 7

To a stirring suspension of (2-{5-[3,4-difluoro-2-(2-fluoro-4-iodo-phenylamino)-phenyl]-[1,3,4]oxadiazol-2-ylamino}-ethyl)-methyl-carbamic acid tert-butyl ester (0.65 mL, 1.103 mmol) in methanol (10 mL) is bubbled in HCl gas for approximately 1 minute. The mixture was allowed to stir at ambient temperature for 1 hour. The reaction mixture was concentrated in vacuo which afforded a yellow oil. Diethyl ether was added and mixture was allowed to stand overnight. The afforded white solids were tritrated several times with diethyl ether and dried in vacuo. Afforded the title compound as a hydrochloride salt (0.58 g, 94.5% based on 1.87 mole equivalence of HCl). m.p.=173-175° C.; ¹NMR (400 MHz; DMSO-d₆) 8.74-8.78 (m, 2H), 8.22 (t, 1H, J=5.6 Hz), 7.60 (dd, 1H, J=10.7, 2.0), 7.53-7.57 (m, 1H), 7.36-7.39 (m, 1H), 7.21-7.28 (m, 1H), 6.73-6.79 (m, 1H), 3.48-3.52 (m, 2H), 3.05-3.10 (m, 2H), 2.52 (t, 3H, J=5.4 Hz); MS(APCI+)=490; Anal. calcd/found for C₁₇H₁₅F₃IN₅O* (1.90 moles) HCl: C 36.56/36.41, H 3.05/3.11, N 12.54/12.14, F 10.20/10.43, Cl 12.06/12.20. C26CPA1 IC₅₀=0.027 μM

### EXAMPLE 19

### N-{5-[3,4-Difluoro-2-(2-fluoro-4-iodo-phenylamino)-phenyl]-[1,3,4]oxadiazol-2-yl}-2,2,N′,N′-tetramethyl-propane-1.3-diamine

m.p.=143-146° C.; ¹NMR (400 MHz; DMSO-d₆) 9.29 (bs, 1H), 8.80 (s, 1H), 8.16 (m, 1H), 7.57-7.65 (m, 2H), 7.41 (d, 1H, J=8.3 Hz), 7.26-7.31 (m, 1H), 6.78-6.79 (m, 1H), 3.23 (d, 2H, J=5.9 Hz), 3.05 (m, 2H), 2.82 (s, 3H), 2.81 (s, 3H), 1.04 (s, 6H); MS(APCI+)=546. C26CPA1 IC₅₀=0.309 μM

### EXAMPLE 20

### {5-[3,4-Difluoro-2-(2-fluoro-4-iodo-phenylamino)-phenyl]-[1,3,4]oxadiazol-2-yl}-(3-methoxy-propyl)-amine

m.p.=67-69° C.; ¹NMR (400 MHz; CD₃OD) 7.57 (ddd, 1H, J=8.5, 5.4, 1.7 Hz), 7.45 (dd, 1H, J=10.7, 2.0), 7.35-7.37 (m, 1H), 7.02 (ddd, 1H, J=16.4, 9.2, 7.2 Hz), 6.69 (ddd, 1H, J=13.9, 8.5, 5.1 Hz), 3.45 (t, 2H, J=5.9 Hz), 3.37 (t, 2H, J=6.8 Hz), 3.29 (s, 3H), 1.86 (quint, 2H, J=6.3 Hz); MS(APCI+)=505; Anal. calcd/found for C₁₈H₁₆F₃IN₄O₂: C 42.88/43.05, H 3.20/3.20, N 11.11/10.86, F 11.30/11.28 C26CPA1 IC₅₀=0.080 μM.

### EXAMPLE 21

### {5-[3,4-Difluoro-2-(2-fluoro-4-iodo-phenylamino)-phenyl]-[1,3,4]oxadiazol-2-yl}-(2-morpholin-4-yl-ethyl)-amine

Step 1

To a stirred suspension of 2,3,4-trifluorobenzoic acid (78 g, 0.44 moles) in dry THF (1.25 L) under nitrogen at −78° C. was added LiHMDS (450 mL, 1 M solution in THF/hexanes) dropwise at such a rate that he temperature was maintained below −67° C. A dark orange solution was formed and this was stirred for another 20 minutes at −67° C. The mixture was designated as Solution A. To a stirred solution of 2-fluoro-4-iodoaniline (105 g, 0.44 moles, Aldrich) in dry THF (1.25 L) under nitrogen at −78° C. was added LiHMDS (450 mL, 1 M solution in THF/hexanes) dropwise at such a rate that he temperature was maintained below −67° C. The dark brown suspension was stirred for an additional 30 minutes at −67° C. The mixture was designated as Solution B. Solution A was transferred to, solution B via a cannula under positive nitrogen pressure at −65° C at such a rate to keep the temperature below −55° C. Then the mixture was slowly warmed to RT and stirred overnight. The reaction mixture was quenched with dry HCl in diethyl ether (1.5 L, freshly prepared, pH ˜1-2. The solution was filtered through a layer of Celite. The filtrate was washed with aq. HCl (2M, 2×3L), brine and dried. Solvent was removed under reduced pressure to give a solid, which was suspended in hexanes-acetone (9:1, v/v, 150 mL) and stirred for 30 minutes. 3,4-difluoro-2-[(2-fluoro-4-iodophenyl)amino] benzoic acid was obtained by filtration as a white solid (135 g, 78%, mp. 195-197° C.).

Step 2

In an oven-dried three-neck, 2 L flask was taken 3,4-difluoro-2-[(2-fluoro-4-iodophenyl)amino] benzoic acid (196.7 g, 0.5 moles) and DMF (900 mL). To this stirred solution was added pyridine (44.4 mL, 43.5 g, 0.55 moles) at RT, and then pentafluorophenyl trifluoroacetate (95 mL, 154 g, 0.55 moles) was added dropwise within 30 minutes. The mixture was stirred at RT for 20 hours. The mixture was diluted with hexanes-diethyl ether (1:1, v/v, 3L) and washed successively with water (2×2L), 1M HCl (2×2L), saturated NaHCO₃ solution (2×2L) and finally with water (2×2L). The organic layer was dried and concentrated under reduced pressure to afford 2,3,4,5,6-pentafluorophenyl-3,4-difluoro-2-[(2-fluoro-4-iodophenyl)amino]benzoate as a red oil (92.3%, 258.5 g).

Step 3

To a stirred solution of anhydrous hydrazine (28.61 g, 0.89 moles in DCM (2L) was added a solution of 2,3,4,5,6-pentafluorophenyl-3,4-difluoro-2-[(2-fluoro-4-iodophenyl)amino]benzoate (250 g, 0.447 moles) in DCM (800 mL) dropwise at 0° C. The mixture was allowed to warm to RT and stirred for 3 hours. The precipitated white solid was collected by filtration, an the filtrate was concentrated to dryness. The solid and the residue were combined and taken into ethyl acetate (4L), washed with water (2×3L), brine (2×3L), dried and concentrated under reduced pressure to approximately 1.5 L. The precipitated solid was re-dissolved by heating the mixture to boiling temperature. Hexane (1L) was added and the solution was kept at RT overnight. N-amino{3,4-difluoro-2-[(2-fluoro-4-iodopheyl)amino]phenyl} carboxamide was obtained as colorless needles (109.5 g) as crop I. The mother liquor was concentrated to 1L to give a second crop as colorless needles (20.2 g). Total 129.7 g in 71.2% yield, mp. 168-169° C.

Step 4

To a solution of N-amino{3,4-difluoro-2-[(2-fluoro-4-iodopheyl)amino]phenyl} carboxamide (50 g, 123 mmoles) in DMF (250 mL) was added 1,1′-carbonyldiimidazole (20.91 g, 129 mmoles, 1.05 eq). The mixture was stirred at RT for 5 hours, then poured_into ethyl acetate (2.5L) and washed with water (2×2.5L), brine (2×2.5 L), dried and evaporated to give 5-[3,4-difluoro-2-(2-fluoro-4-iodo-phenylamino)-phenyl]-3H-[1,3,4]oxadiazol-2-one as a white solid (53.2 g). Further crystallization from THF-hexanes gave the pure product as colorless needles in 96% yield, 51 g, mp. 224-225° C.

Step 5

To a stirring suspension of 5-[3,4-difluoro-2-(2-fluoro-4-iodo-phenylamino)-phenyl]-3H-[1,3,4]oxadiazol-2-one (1.055 g, 2.436 mmol) in ethanol (25 mL) was added 2-morpholin-4-yl-ethylamine (0.96 mL, 7.307 mmol) and heated to reflux (oil bath temperature was set to 100° C.). After reaction refluxed for 20 hours the mixture was allowed to cool and concentrated in vacuo. The afforded residue was dissolved in ethyl acetate and partitioned saturated NaHCO₃ solution and twice with brine. Organics were collected and dried over Na₂SO₄, filtered and concentrated in vacuo. Afforded 4′-(2-morpholin-4-yl-ethylamine)-1′-[3,4-difluoro-2-(2-fluoro-4-iodo-phenylamino)]-semicarbazole as a light brown foam/solid (1.06 g, 77%).

Step 6

To a stirring solution of 4′-(2-morpholin-4-yl-ethylamine)-1′-[3,4-difluoro-2-(2-fluoro-4-iodo-phenylamino)]-semicarbazole (0.911 g, 1.617 mmol) in dichloromethane (40 mL) was added resin bound triphenylphosphine (2.84 g, 4.852 mmol), triethylamine (1.80 mL, 12.936 mmol) and carbon tetrachloride (0.39 mL, 4.852 mmol) and heated to reflux (oil bath set at 46° C.). After 3.5 hours the reaction mixture was allowed to cool and the reaction mixture was filtered and the resin was rinsed with 2:1 dichloromethane/methanol (100 mL). The filtrate was collected and concentrated in vacuo. The affording residue was dissolved in ethyl acetate and partitioned with water. Organics were washed twice with water and twice with saturated NaHCO₃ and once with brine. Organics were collected and dried over Na₂SO₄, filtered and concentrated in vacuo. Silica column purification was performed with 2% methanol in dichloromethane and afforded the title compound as an off-white foam/solid (0.380 g, 43%).

m.p.=77-80° C.; ¹NMR (400 MHz CD₃OD) 7.60 (ddd, 1H, J=8.8, 5.4, 2.0 Hz), 7.48 (dd, 1H, J=10.5, 1.7 Hz), 7.39 (ddd, 1H, J=8.8, 2.0, 1.2 Hz), 6.72 (ddd, 1H, J=14.0, 8.8, 5.6 Hz), 3.67 (t, 4H, J=4.6 Hz), 3.46 (t, 2H, J=6.3 Hz), 2.61 (t, 2H, J=6.3 Hz), 2.50-2.52 (m, 4H); MS(APCI+)=546; Anal. calcd/found for C₂₀H₁₉F₃IN₅O₂: C 44.05/43.09, H 3.51/3.16, N 12.84/12.51, F 10.45/10.26.

C26CPA1 IC₅₀=0.032 μM

### EXAMPLE 22

### (2,3-Difluoro-6-{5-[4-(2-methoxy-ethyl)-piperazin-1-yl]-[1,3,4]oxadiazol-2-yl}-phenyl)-(2-fluoro-4-iodo-phenyl)-amine

m.p.=63-65° C.; ¹NMR (400 MHz; CD₃OD) 7.64-7.67 (m, 1H), 7.47 (dd, 1H, J=10.5, 1.5 Hz), 7.37 (dd, 1H, J=8.4, 1.2 Hz), 7.06 (dd, 1H, J=16.4, 9.0 Hz), 6.68 (ddd, 1H, J=13.7, 8.8, 4.9 Hz), 3.53-3.57 (cm, 6H), 3.34 (s, 3H), 2.61-2.64 (cm, 6H); MS(APCI+)=560; Anal. calcd/found for C₂₁H₂₁F₃IN₅O₂: C 45.10/45.39, H 3.78/3.68, N 12.52/12.17, F 10.19/10.25. C26CPA1 IC₅₀=0.130 μM

### EXAMPLE 23

### 5-{5-[3,4-Difluoro-2-(2-fluoro-4-iodo-phenylamino)-phenyl]-[1,3,4]oxadiazol-2-ylamino}-2,2-dimethyl-pentan-1-ol

m.p.=67-70° C.; ¹NMR (400 MHz; DMSO-d₆) 8.78 (s, 1H), 7.92 (t, 1H, J=5.6 Hz), 7.60 (d, 1H, J=2.0 Hz), 7.51-7.54 (m, 1H), 7.38 (d, 1H, J=9.3), 7.21-7.27 (m, 1H), 6.73-6.78 (m, 1H), 4.42 (t, 1H, J=5.4 Hz), 3.13 (q, 2H, J=13.0, 6.1 Hz), 3.04 (d, 2H, J=5.4 Hz), 1.42-1.48 (m, 2H), 1.14-1.20 (m, 2H), 0.74 (s, 6H); MS(APCI+)=547; Anal. calcd/found for C₂₁H₂₂F₃IN₄O₂ with 0.12 moles of residual C₆H₁₅N: C 46.71/47.11, H 4.30/4.25, N 10.33/9.97, F 10.21/10.29. C26CPA1 IC₅₀=0.292 μM

### EXAMPLE 24

### 1-{5-[3.4-Difluoro-2-(2-fluoro-4-iodo-phenylamino)-phenyl]-[1,3,4]oxadiazol-2-yl}-pyrrolidin-3-ol

m.p.=182-183° C.; ¹NMR (400 MHz; CD₃OD) 7.63-7.67 (m, 1H), 7.47 (dd, 1H, J=10.7, 2.0), 7.36-7.39 (m, 1H), 7.03-7.09 (m, 1H), 6.67-6.73 (m, 1H), 4.52-4.53 (m, 1H), 3.64-3.69 (m, 3H), 3.48-3.51 (m, 1H), 2.06-2.17 (m, 2H); MS(APCI+)=503; Anal. calcd/found for C₁₈H₁₄F₃IN₄O₂ with 0.04 moles of residual C₆H₁₄: C 43.32/43.71, H 2.90/2.79, N 11.08/10.85, F 11.27/11.22. C26CPA1 IC₅₀=0.170 μM

### EXAMPLE 25

### (1-{5-[3,4-Difluoro-2-(2-fluoro-4-iodo-phenylamino)-phenyl]-[1,3,4]oxadiazol-2-yl}-piperidin-4-yl)-methanol

m.p.=90-92° C.; ¹NMR (400 MHz; DMSO-d₆) 8.67 (s, 1H), 7.68-7.70 (m, 1H), 7.59 (dd, 1H, J=11.0, 2.0 Hz), 7.37 (d, 1H, J=8.8 Hz), 7.23-7.29 (m, 1H), 6.68-6.73 (m, 1H), 4.51 (t, 1H, J=5.1 Hz), 3.85-3.98 (m, 2H), 3.22-3.27 (m, 2H), 2.98-3.03 (m, 2H), 1.68-1.72(m, 2H), 1.57 (m, 1H), 1.12-1.20 (m, 2H); MS(APCI+)=53; Anal. calcd/found for C₂₀H₁₈F₃IN₄O₂ with 0.05 moles of residual C₄H₈O₂: C 45.38/45.58, H 3.47/3.56, N 10.48/10.09, F 10.66/10.50. C26CPA1 IC₅₀=>1.0 μM

### EXAMPLE 26

### 2-(1-{5-[3,4-Difluoro-2-(2-fluoro-4-iodo-phenylamino)-phenyl]-[1,3,4]oxadiazol-2-yl}-piperidin-4-yl)-ethanol

m.p.=121-122° C.; ¹NMR (400 MHz; CD₃OD) 7.61-7.65 (m, 1H), 7.45 (dd, 1H, J=10.5, 2.0 Hz), 7.33-7.35 (m, 1H), 7.01-7.07 (m, 1H), 6.63-6.68 (m, 1H), 3.94-3.97 (m, 2H), 3.60 (t, 2H, J=6.6 Hz), 3.05-3.09 (m, 2H), 1.81 (m, 2H), 1.78 (m, 1H), 1.49 (q, 2H, J=13.2, 6.6 Hz), 1.24-1.28 (m, 2H); MS(APCI+)=545; Anal. calcd/found for C₂₁H₂₀F₃IN₄O₂ with 0.01 moles of residual C₄H₈O₂: C 46.35/46.48, H 3.71/3.60, N 10.28/9.86, F 10.45/10.85. C26CPA1 IC₅₀=0.140 μM

### EXAMPLE 27

### 4-({5-[3,4-Difluoro-2-(2-fluoro-4-iodo-phenylamino)-phenyl]-[1,3,4]oxadiazol-2-yl}-ethyl-amino)-butan-1-ol

m.p.=121-122° C.; ¹NMR (400 MHz; CD₃OD) 7.61-7.65 (m, 1H), 7.46 (dd, 1H, J=10.5, 2.0), 7.35-7.38 (m, 1H), 7.03-7.08 (m, 1H), 6.64-6.70 (m, 1H), 3.57 (t, 2H, J=6.3), 3.45-3.53 (m, 4H), 1.68-1.76 (m, 2H), 1.51-1.58 (m, 2H), 1.23 (t, 2H, J=7.1 Hz); MS(APCI+)=533; Anal. calcd/found for C₂₀H₂₀F₃IN₄O₂: C 45.13/45.29, H 3.79/3.79, N 10.53/10.35, F 10.71/10.76. C26CPA1 IC₅₀=0.180 μM

### EXAMPLE 28

### 1-{5-[3,4-Difluoro-2-(2-fluoro-4-iodo-phenylamino)-phenyl]-[1,3,4]oxadiazol-2-yl}-piperidin-4-ol

m.p.=216-217° C.; ¹NMR (400 MHz; DMSO-d₆) 8.62 (s, 1H), 7.64-7.67 (m, 1H), 7.32-7.34 (m, 1H), 7.19-7.29 (m, 1H), 6.64-6.69 (m, 1H), 4.76 (d, 1H, J=3.9 Hz), 3.60-3.67 (m, 3H), 3.14-3.20 (m, 2H), 1.70-1.75 (m, 2H), 1.32-1.41 (m, 2H); MS(APCI+)=517; HPLC=9.013 min at 254 nm. C26CPA1 IC₅₀=0.074 μM

### EXAMPLE 29

### 2-{5-[3,4-Difluoro-2-(2-fluoro-4-iodo-phenylamino)-phenyl]-[1,3,4]oxadiazol-2-ylamino}-ethanol

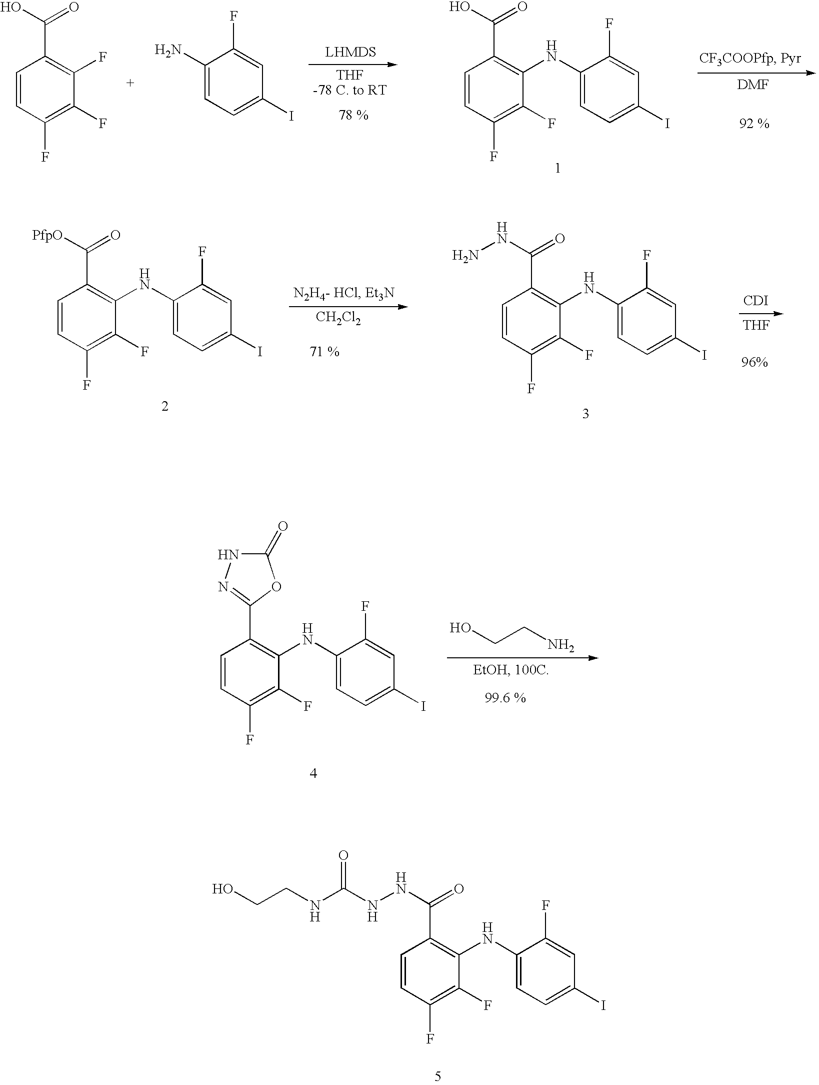

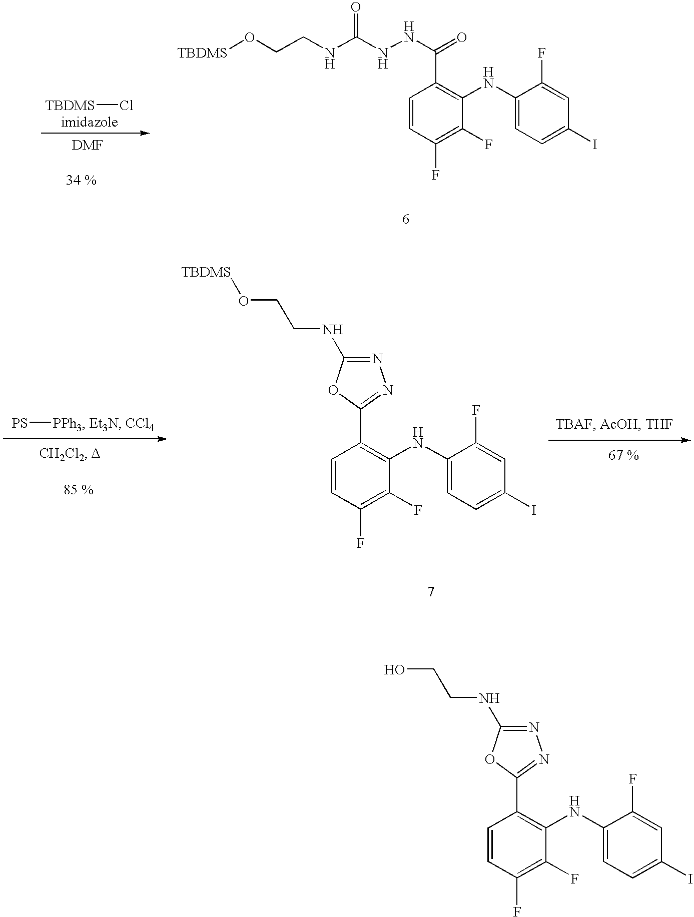

Step 1

To a stirred suspension of 2,3,4-trifluorobenzoic acid (78 g, 0.44 moles) in dry THF (1.25 L) under nitrogen at −78° C. was added LiHMDS (450 mL, 1 M solution in THF/hexanes) dropwise at such a rate that he temperature was maintained below −67° C. A dark orange solution was formed and this was stirred for another 20 minutes at −67° C. The mixture was designated as Solution A. To a stirred solution of 2-fluoro-4-iodoaniline (105 g, 0.44 moles, Aldrich) in dry THF (1.25 L) under nitrogen at −78° C. was added LiHMDS (450 mL, 1 M solution in THF/hexanes) dropwise at such a rate that he temperature was maintained below −67° C. The dark brown suspension was stirred for an additional 30 minutes at −67° C. The mixture was designated as Solution B. Solution A was transferred to solution B via a cannula under positive nitrogen pressure at −65° C. at such a rate to keep the temperature below −55° C. Then the mixture was slowly warmed to RT and stirred overnight. The reaction mixture was quenched with dry HCl in diethyl ether (1.5 L, freshly prepared, pH ˜1-2. The solution was filtered through a layer of Celite. The filtrate was washed with aq. HCl (2M, 2×1L), brine and dried. Solvent was removed under reduced pressure to give a solid, which was suspended in hexanes-acetone (9:1, v/v, 150 mL) and stirred for 30 minutes. 3,4-difluoro-2-[(2-fluoro-4-iodophenyl)amino] benzoic acid was obtained by filtration as a white solid (135 g, 78%, mp. 195-197° C.).

Step 2

In an oven-dried three-neck, 2 L flask was taken 3,4-difluoro-2-[(2-fluoro-4-iodophenyl)amino] benzoic acid (196.7 g, 0.5 moles) and DMF (900 mL). To this stirred solution was added pyridine (44.4 mL, 43.5 g, 0.55 moles) at RT, and then pentafluorophenyl trifluoroacetate (95 mL, 154 g, 0.55 moles) was added dropwise within 30 minutes. The mixture was stirred at RT for 20 hours. The mixture was diluted with hexanes-diethyl ether (1:1, v/v, 3L) and washed successively with water (2×2L), 1M HCl (2×2L), saturated NaHCO₃ solution (2×2L) and finally with water (2×2L). The organic layer was dried and concentrated under reduced pressure to afford 2,3,4,5,6-pentafluorophenyl-3,4-difluoro-2-[(2-fluoro-4-iodophenyl)amino]benzoate as a red oil (92.3%, 258.5 g).

Step 3

To a stirred solution of anhydrous hydrazine (28.61 g, 0.89 moles in DCM (2L) was added a solution of 2,3,4,5,6-pentafluorophenyl-3,4-difluoro-2-[(2-fluoro-4-iodophenyl)amino]benzoate (250 g, 0.447 moles) in DCM (800 mL) dropwise at 0° C. The mixture was allowed to warm to RT and stirred for 3 hours. The precipitated white solid was collected by filtration, an the filtrate was concentrated to dryness. The solid and the residue were combined and taken into ethyl acetate (4L), washed with water (2×3L), brine (2×3L), dried and concentrated under reduced pressure to approximately 1.5 L. The precipitated solid was re-dissolved by heating the mixture to boiling temperature. Hexane (IL) was added and the solution was kept at RT overnight. N-amino{3,4-difluoro-2-[(2-fluoro-4-iodopheyl)amino]phenyl} carboxamide was obtained as colorless needles (109.5 g) as crop I. The mother liquor was concentrated to 1L to give a second crop as colorless needles (20.2 g). Total 129.7 g in 71.2% yield, mp. 168-169° C.

Step 4

To a solution of N-amino{3,4-difluoro-2-[(2-fluoro-4-iodopheyl)amino]phenyl} carboxamide (50 g, 123 mmoles) in DMF (250 mL) was added 1,1′-carbonyldiimidazole (^{20.91} g, 129 mmoles, 1.05 eq). The mixture was stirred at RT for 5 hours, then poured into ethyl acetate (2.5L) and washed with water (2×2.5L), brine (2×2.5 L), dried and evaporated to give 5-[3,4-difluoro-2-(2-fluoro-4-iodo-phenylamino)-phenyl]-3H-[1,3,4]oxadiazol-2-one as a white solid (53.2 g). Further crystallization from THF-hexanes gave the pure product as colorless needles in 96% yield, 51 g, mp. 224-225° C.

Step 5

To a stirring suspension of 5-[3,4-difluoro-2-(2-fluoro-4-iodo-phenylamino)-phenyl]-3H-[1,3,4]oxadiazol-2-one (2.02 g, 4.664 mmol) in ethanol (25 mL) was added 2-amino-ethanol (0.84 mL, 13.99 mmol) and heated to reflux (oil bath temperature was set to 100° C.). After reaction refluxed for 16-20 hours, the mixture was allowed to cool and concentrated in vacuo. The afforded residue was dissolved in ethyl acetate and partitioned with sat. NaHCO₃. Organics were washed twice with brine then collected and dried over Na₂SO₄, filtered and concentrated in vacuo. Afforded of 4′-(2-amino-ethanol)-1′-[3,4-difluoro-2-(2-fluoro-4-iodo-phenylamino)]-semicarbazole as a white solid (2.24 g, 97.2%).

Step 6

To a stirring solution of the product 4′-(2-amino-ethanol)-1′-[3,4-difluoro-2-(2-fluoro-4-iodo-phenylamino)]-semicarbazole (2.23 g, 4.512 mmol) in DMF (25 mL) was added imidazole (0.30 g, 4.963 mmol) and tert-butyldimethylsilylchloride (0.748 g, 4.963 mmol). After stirring for 5 hours, the reaction mixture was poured in 25 mL of 1 molar HCl solution and partitioned with ethyl acetate. Organics were washed twice with water and twice with brine. Organics were washed twice with brine then collected and dried over Na₂SO₄, filtered and concentrated in vacuo. Column chromatography of the crude material in 2:1 hexanes/ethyl acetate to 1:1 hexanes/ethyl acetate, afforded 4′-(2-(tert-butyl-dimethyl-silanyloxy)-ethylamine)-1′-[3,4-difluoro-2-(2-fluoro-4-iodo-phenylamino)]-semicarbazole as a white foam/solid (0.93 g, 33.9%).

Step 7

To a stirring solution of product 4′-(2-(tert-butyl-dimethyl-silanyloxy)-ethylamine)-1′-[3,4-difluoro-2-(2-fluoro-4-iodo-phenylamino)]-semicarbazole (0.93 g, 1.528 mmol) in dichloromethane (40 mL) was added resin bound triphenylphosphine# (2.68 g, 4.584 mmol)**, triethylamine (1.7 mL, 12.22 mmol) and carbon tetrachloride (0.37 mL, 4.584 mmol) and the mixture was heated to reflux (oil bath was set at 46° C.). Reaction was complete after 2.5 hours. The reaction mixture was filtered and the resin was rinsed with 50 mL of 1:1 methanol/dichloromethane. The filtrate was collected and concentrated in vacuo. To the afforded crude material was added ethyl acetate and partitioned with water. Organics were washed several times with water and twice with brine. Organics were collected and dried over Na₂SO₄, filtered and concentrated in vacuo which afforded [2-(tert-butyl-dimethyl-silanyloxy)-ethyl]-{5-[3,4-difluoro-2-(2-fluoro-4-iodo-phenylamino)-phenyl]-[1,3,4]oxadiazol-2-yl}-amine as a yellow foam/solid (0.77 g, 85.4%). # triphenylphosphine can also be used, but will then require column chromatography. **2.0 mole equivalence of PS-PPh₃ is sufficient for reaction completion.

Step 8

To a stirring solution of [2-(tert-butyl-dimethyl-silanyloxy)-ethyl]-{5-[3,4-difluoro-2-(2-fluoro-4-iodo-phenylamino)-phenyl]-[1,3,4]oxadiazol-2-yl}-amine (0.76 g, 1.287 mmol) in THF (7 mL) at 0° C. was added acetic acid (0.074 mL, 1.287 mmol) and tert-butylammoniumfluoride (TBAF in THF, 1.0M solution, 1.9 mL, 1.917 mmol) and reaction was allowed to warm to ambient temperature and stirred for 6 hours. The reaction mixture was partitioned between ethyl acetate and water. Organics were washed twice with saturated NaHCO₃ solution and once with 0.5 M HCl solution. Organics were collected and dried over Na₂SO₄, filtered and concentrated in vacuo which afforded a white foam. The crude material was crystallized from dichloromethane and the solids were dried in vacuo. To afford 2-{5-[3,4-difluoro-2-(2-fluoro-4-iodo-phenylamino)-phenyl]-[1,3,4]oxadiazol-2-ylamino}-ethanol as yellow crystals (0.409 g, 66.7%). m.p.=100-103° C.; ¹NMR (400 MHz; CD₃OD) 7.57-7.61 (m, 1H), 7.46 (dd, 1H, J=10.5, 2.0 Hz), 7.36-7.39 (m, 1H0, 7.01-7.06 (m, 1H), 6.68-6.74 (m, 1H), 3.71 (t, 2H, J=5.6 Hz), 3.42 (t, 2H, J=5.6 Hz); MS(APCI+)=477; Anal. calcd/found for C₁₆H₁₂F₃IN₄O₂: C 40.36/40.10, H 2.54/2.32, N 11.77/11.44, F 11.97/12.19. C26CPA1 IC₅₀=0.015 μM

### EXAMPLE 30

### 3-{5-[3,4-Difluoro-2-(2-fluoro-4-iodo-phenylamino)-phenyl]-[1,3,4]oxadiazol-2-ylamino}-propan-1-ol

m.p.=154-155° C.; ¹NMR (400 MHz; CD₃OD) 7.57-7.62 (m, 1H), 7.47 (dd, 1H, J=10.7, 2.0 Hz), 7.37-7.39 (M, 1H), 7.00-7.07 (m, 1H), 6.69-6.74 (m, 1H),.3.64 (t, 2H, J=6.1 Hz), 3.41 (t, 2H, J=6.8 Hz), 1.84 (quint, 2H, J=6.6 Hz); MS(APCI+)=491; Anal. calcd/found for C₁₇H₁₄F₃IN₄O₂: C 41.57/41.67, H 3.08/2.81,N 11.41/11.31. C26CPA1 IC₅₀=0.028CM

### EXAMPLE 31

### 4-{5-[3,4-Difluoro-2-(2-fluoro-4-iodo-phenylamino)-phenyl]-[1,3,4]oxadiazol-2-ylamino}-butan-1-ol

m.p.=82-83° C.; ¹NMR (400 MHz; CD₃OD) 7.57-7.61 (m, 1H), 7.47 (dd, 1H, J=10.5, 2.0), 7.36-7.39 (m, 1H), 7.00-7.07 (m, 1H), 3.56 (t, 2H, J=6.4 Hz), 3.31-3.34 (m, 2H), 1.56-1.74 (m, 4H); MS(APCI+)=505; Anal. calcd/found for C₁₈H₁₆F₃IN₄O₂ with 0.27 moles residual CH₂Cl₂: C 41.62/41.24, H 3.16/3.00, N 10.63/10.39, F 10.81/10.71. C26CPA1 IC₅₀=0.012 μM

### EXAMPLE 32

### 5-{5-[3,4-Difluoro-2-(2-fluoro-4-iodo-phenylamino)-phenyl]-[1,3,4]oxadiazol-2-ylamino}-pentan-1-ol

m.p.=119-121° C.; ¹NMR (400 MHz; CD₃OD) 7.56-7.60 (m, 1H), 7.47 (dd, 1H, J=10.5, 2.0 Hz), 7.37-7.48 (m, 1H), 7.00-7.06 (m, 1H), 6.68-6.74 (m, 1H), 3.54 (t, 2H, J=6.4 Hz), 3.31-3.32 (m, 2H), 1.62-1.69 (m, 2H), 1.53-1.60 (m, 2H), 1.40-1.48 (m, 2H); MS(APCI+)=519; Anal. calcd/found for C₁₉H₁₈F₃IN₄O₂:C 44.03/44.23, H 3.50/3.36, N 10.81/10.61, F 11.00/11.28. C26CPA1 IC₅₀=0.120 μM
Procedure for the synthesis of Examples 33-66

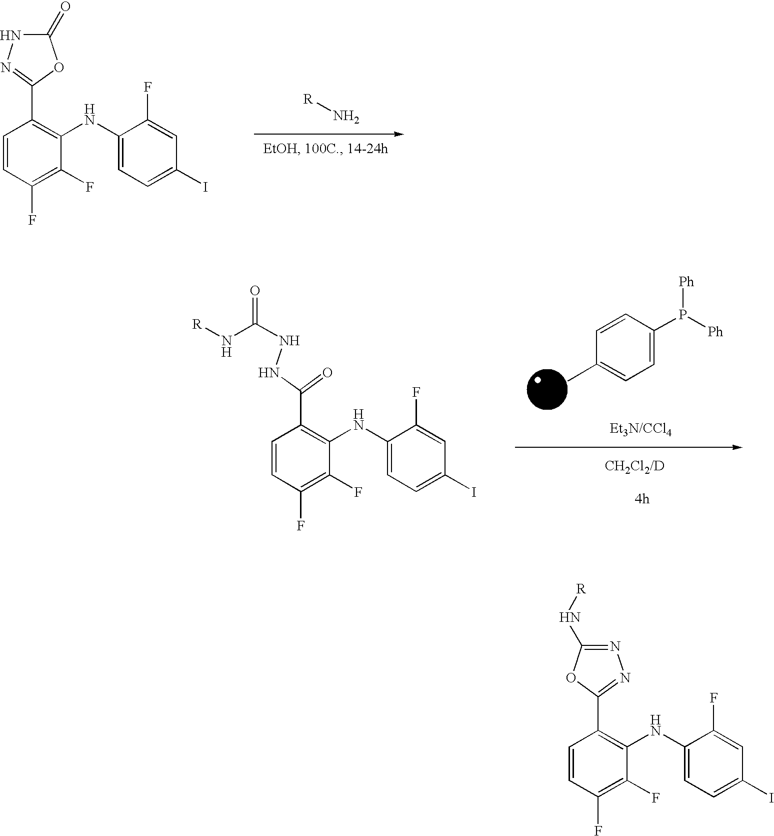

Examples 33-66 were prepared utilizing combinatorial synthetic methods as detailed below, by the combination of the respective amine with 3H-oxadiazol-2-one as prepared above.

General Procedure:

Step A:

5-[3,4-difluoro-2-(2-fluoro-4-iodo-phenylamino)-phenyl]-3H-[1,3,4]oxadiazol-2-one (0.022 g, 0.05 mmole) was dispensed into each 2 dram vial as a solution in TM and solvent evaporated. Ethanol (2 mL) is added followed by the appropriate amine (0.10 mmol). The vials were sealed with teflon caps and allowed to shake on an orbital shaker at 100° C. for 14-24 hours. The reaction mixtures were then concentrated.

Step B:

To each of the vials containing the product from Step A was added dichloromethane (2 mL), triphenylphosphine resin (0.087 g, 0.15 mmol, 1.73 mmol/g, Argonaut Technologies), triethylamine (0.056 mL, 0.40 mmol) and carbon tetrachloride (0.012 mL, 0.15 mmol). Each vial was sealed with a teflon-coated cap and shaken at 45° C. for 4 hours. The reactions were filtered through a SPEC 3A disc filter, washed with 2 mL of dichloromethane and concentrated. Purification was performed via HPLC using a YMC 30×100 mm ODS-A (C18) 5 micron column. Solvent system consisted of acetonitrile with 3% 1-propanol (A) and water with 3% 1-propanol (B) with a flow rate of 30 mL/min. Mobile phase was 20%A, 80% B from 0-1 minute, 20% A, 80% B to 100% A from 1-5.5 minutes, followed by 100% A from 5.5-10.0 minutes. The desired fractions were collected and dried in vacuo. Afforded the desired 5-[3,4-difluoro-2-(2-fluoro-4-iodo-phenylamino)-phenyl]-[1,3,4]oxadiazole-2-substituted amine.

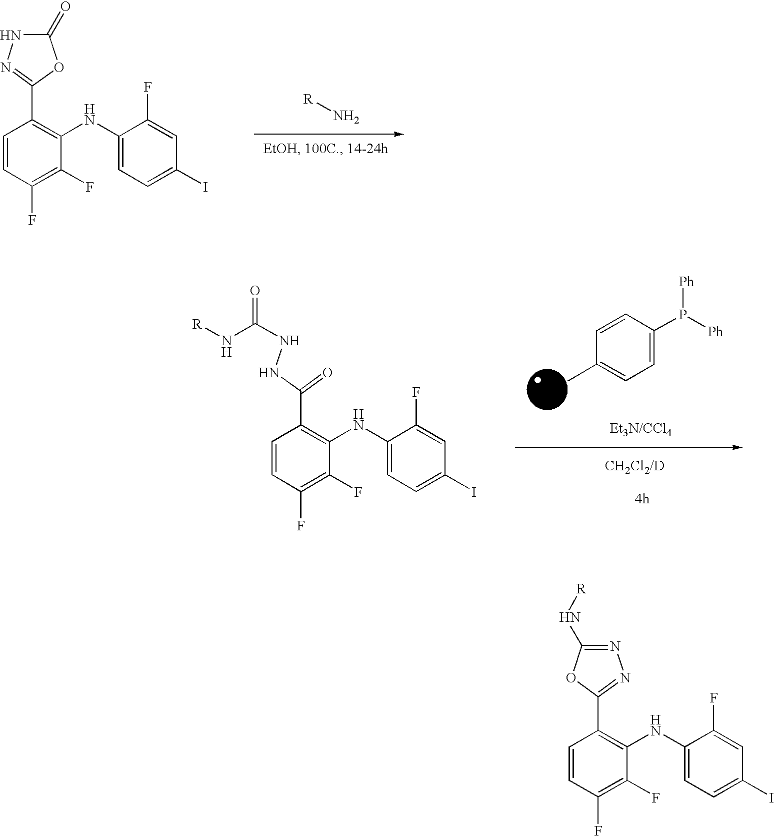

### EXAMPLE 33

### {5-[3,4-Difluoro-2-(2-fluoro-4-iodo-phenylamino)-phenyl]-[1,3,4]oxadiazol-2-yl}-(2-methoxy-ethyl)-amine

MS(APCI+)=491. C26CPA1% Inhibition @ 1 μM =57.7%. C26CPA1% Inhibition @ 0.1 μM=0%

### EXAMPLE 34

### {5-[3,4-Difluoro-2-(2-fluoro-4-iodo-phenylamino)-phenyl]-[1,3,4]oxadiazol-2-yl}-(2-phenoxy-ethyl)-amine

MS(APCI+)=553

C26CPA1% Inhibition @ 1 μM=69% C26CPA1% Inhibition @ 0.1 μM=0%

### EXAMPLE 35

### {5-[3,4-Difluoro-2-(2-fluoro-4-iodo-phenylamino)-phenyl]-[1,3,4]oxadiazol-2-yl}-(2-pyridin-4-yl-ethyl)-amine

MS(APCI+)=538

C26CPA1% Inhibition @ 1 μM=74.9%

C26CPA1% Inhibition @ 0.1 μM=0%

### EXAMPLE 36

### Butyl-{5-[3,4-difluoro-2-(2-fluoro-4-iodo-phenylamino)-phenyl]-[1,3,4]oxadiazol-2-yl}-amine

MS(APCI+)=489

C26CPA1% Inhibition @ 1 μM=67%

C26CPA1% Inhibition @ 0.1 μM=23.8%

### EXAMPLE 37

### {5-[3,4-Difluoro-2-(2-fluoro-4-iodo-phenylamino)-phenyl]-[1,3,4]oxadiazol-2-yl}-(5-methyl-furan-2-ylmethyl)-amine

MS(APCI+)=527

C26CPA1% Inhibition @ 1 μM=87.2%

C26CPA1% Inhibition @ 0.1 μM=18.4%

### EXAMPLE 38

### {5-[3,4-Difluoro-2-(2-fluoro-4-iodo-phenylamino)-phenyl]-[1,3,4]oxadiazol-2-yl}-(2-thiophen-2-yl-ethyl)-amine

MS(APCI+)=543

C26CPA1% Inhibition @ 1 μM=65.1%

C26CPA1% Inhibition @ 0.1 μM=0%

### EXAMPLE 39

### {5-[3,4-Difluoro-2-(2-fluoro-4-iodo-phenylamino)-phenyl]-[1,3,4]oxadiazol-2-yl}-[2-(3-methoxy-phenyl)-ethyl]-amine

MS(APCI+)=567

C26CPA1% Inhibition @ 1 μM=40.2%

C26CPA1% Inhibition @ 0.1 μM=0%

### EXAMPLE 40

### {5-[3,4-Difluoro-2-(2-fluoro-4-iodo-phenylamino)-phenyl]-[1,3,4]oxadiazol-2-yl}-[2-(3,4-dimethoxy-phenyl)-ethyl]-amine

MS(APCI+)=597

C26CPA1% Inhibition @ 1 μM=26.2%

C26CPA1% Inhibition @ 0.1 μM=0%

### EXAMPLE 41

### {5-[3,4-Difluoro-2-(2-fluoro-4-iodo-phenylamino)-phenyl]-[1,3,4]oxadiazol-2-yl}-[2-(2,5-dimethoxy-phenyl)-ethyl]-amine

MS(APCI+)=597

C26CPA1% Inhibition @ 1 μM=29.8%

C26CPA1% Inhibition @ 0.1 μM=0%

### EXAMPLE 42

### {5-[3,4-Difluoro-2-(2-fluoro-4-iodo-phenylamino)-phenyl]-[1,3,4]oxadiazol-2-yl}-[2-(3,4-dimethoxy-phenyl)-ethyl]-methyl-amine

MS(APCI+)=611

C26CPA1% Inhibition @ 1 μM=10% C26CPA1% Inhibition @ 0.1 μM=8.3%

### EXAMPLE 43

### {5-[3,4-Difluoro-2-(2-fluoro-4-iodo-phenylamino)-phenyl]-[1,3,4]oxadiazol-2-yl}-methyl-(2-pyridin-4-yl-ethyl)-amine

MS(APCI+)=552

C26CPA1% Inhibition @ 1 μM=31.7%

C26CPA1% Inhibition @ 0.1 μM=26.6%

### EXAMPLE 44

### {5-[3,4-Difluoro-2-(2-fluoro-4-iodo-phenylamino)-phenyl]-[1,3,4]oxadiazol-2-yl}-methyl-(2-pyridin-2-yl-ethyl)-amine

MS(APCI+)=552

C26CPA1% Inhibition @ 1 μM=19%

C26CPA1% Inhibition @ 0.1 μM=5.2%

### EXAMPLE 45

### {5-[3,4-Difluoro-2-(2-fluoro-4-iodo-phenylamino)-phenyl]-[1,3,4]oxadiazol-2-yl}-propyl-amine

MS(APCI+)=475

C26CPA1% Inhibition @ 1 μM=31.4%

C26CPA1% Inhibition @ 0.1 μM=3.1%

### EXAMPLE 46

### sec-Butyl-{5-[3,4-difluoro-2-(2-fluoro-4-iodo-phenylamino)-phenyl]-[1,3,4]oxadiazol-2-yl}-amine

MS(APCI+)=489

C26CPA1% Inhibition @ 1 μM=31.6%

C26CPA1% Inhibition @ 0.1 μM=31.2%

### EXAMPLE 47

### {5-[3,4-Difluoro-2-(2-fluoro-4-iodo-phenylamino)-phenyl]-[1,3,4]oxadiazol-2-yl}-(2,2-dimethyl-propyl)-amine

MS(APCI+)=503

C26CPA1% Inhibition @ 1 μM=43.2%

C26CPA1% Inhibition @ 0.1 μM=18.9%

### EXAMPLE 48

### {5-[3,4-Difluoro-2-(2-fluoro-4-iodo-phenylamino)-phenyl]-[1,3,4]oxadiazol-2-yl}-isobutyl-amine

MS(APCI+)=489

C26CPA1% Inhibition @ 1 μM=66.9%

C26CPA1% Inhibition @ 0.1 μM=31.7%

### EXAMPLE 49

### {5-[3,4-Difluoro-2-(2-fluoro-4-iodo-phenylamino)-phenyl]-[1,3,4]oxadiazol-2-yl}-(1,2,2-trimethyl-propyl)-amine

MS(APCI+)=517

C26CPA1% Inhibition @ 1 μM=0%

C26CPA1% Inhibition @ 0.1 μM=0%

### EXAMPLE 50

### {5-[3,4-Difluoro-2-(2-fluoro-4-iodo-phenylamino)-phenyl]-[1,3,4]oxadiazol-2-yl}-(2-methyl-butyl)-amine

MS(APCI+)=503

C26CPA1% Inhibition @ 1 μM=15.2%

C26CPA1% Inhibition @ 0.1 μM=0%

### EXAMPLE 51

### Bicyclo[2,2,1]hept-2-yl-{5-[3,4-difluoro-2-(2-fluoro-4-iodo-phenylamino)-phenyl]-[1,3,4]oxadiazol-2-yl}-amine

MS(APCI+)=527

C26CPA1% Inhibition @ 1 μM=96% C26CPA1% Inhibition @ 0.1 μM=13.1%

### EXAMPLE 52

### {5-[3,4-Difluoro-2-(2-fluoro-4-iodo-phenylamino)-phenyl]-[1,3,4]oxadiazol-2-yl}-[2-(1-methyl-1H-pyrrol-2-yl)-ethyl]-amine

MS(APCI+)=540

C26CPA1 IC₅₀=0.086 μM

### EXAMPLE 53

### {5-[3,4-Difluoro-2-(2-fluoro-4-iodo-phenylamino)-phenyl]-[1,3,4]oxadiazol-2-yl}-[2-(1-methyl-1H-imidazol-4-yl)-ethyl]-amine

MS(APCI+)=541

C26CPA1 IC₅₀=0.230 μM

### EXAMPLE 54

### {2,3-Difluoro-6-[5-(2-pyrrolidin-1-ylmethyl-pyrrolidin-1-yl)-[1,3,4]oxadiazol-2-yl]-phenyl}-(2-fluoro-4-iodo-phenyl)-amine

MS(APCI+)=570

C26CPA1% Inhibition @ 1 μM=30.8%

C26CPA1% Inhibition @ 0.1 μM=21.7%

### EXAMPLE 55

### {5-[3,4-Difluoro-2-(2-fluoro-4-iodo-phenylamino)-phenyl]-[1,3,4]oxadiazol-2-yl}-(2-methoxy-1-methyl-ethyl)-amine

MS(APCI+)=505

C26CPA1% Inhibition @ 1 μM=25.2%

C26CPA1% Inhibition @ 0.1 μM=23.9%

### EXAMPLE 56

### {5-[3,4-Difluoro-2-(2-fluoro-4-iodo-phenylamino)-phenyl]-[1,3,4]oxadiazol-2-yl}-(3-ethoxy-propyl)-amine

MS(APCI+)=519

C26CPA1 IC₅₀=0.044 μM

### EXAMPLE 57

### 3-{5-[3,4-Difluoro-2-(2-fluoro-4-iodo-phenylamino)-phenyl]-[1,3,4]oxadiazol-2-ylamino}-dihydro-furan-2-one

MS(APCI+)=517

C26CPA1% Inhibition @ 1 μM=51.8%

C26CPA1% Inhibition @ 0.1 μM=31.3%

### EXAMPLE 58

### 3-{5-[3,4-Difluoro-2-(2-fluoro-4-iodo-phenylamino)-phenyl]-[1,3,4]oxadiazol-2-ylamino}-propionic Acid Ethyl Ester

MS(APCI+)=533

C26CPA1% Inhibition @ 1 μM=17.8% C26CPA1% Inhibition @ 0.1 μM=0%

### EXAMPLE 59

### {5-[3,4-Difluoro-2-(2-fluoro-4-iodo-phenylamino)-phenyl]-[1,3,4]oxadiazol-2-yl}-[2-(2,6-dimethyl-phenoxy)-1-methyl-ethyl]-amine

MS(APCI+)=595

C26CPA1% Inhibition @ 1 μM=0%

C26CPA1% Inhibition @ 0.1 μM=0%

### EXAMPLE 60

### (1-{5-[3,4-Difluoro-2-(2-fluoro-4-iodo-phenylamino)-phenyl]-[1,3,4]oxadiazol-2-yl}-pyrrolidin-3-yl)-diethyl-amine

MS(APCI+)=558

C26CPA1% Inhibition @ 1 μM=68%

C26CPA1% Inhibition @ 0.1 μM=8.2%

### EXAMPLE 61

### {2,3-Difluoro-6-[5-(4-methyl-[1,4]diazepan-1-yl)-[1,3,4]oxadiazol-2-yl]-phenyl}-(2-fluoro-4-iodo-phenyl)-amine

MS(APCI+)=530

C26CPA1 IC₅₀=0.850 μM

### EXAMPLE 62

### {5-[3,4-Difluoro-2-(2-fluoro-4-iodo-phenylamino)-phenyl]-[1,3,4]oxadiazol-2-yl}-[2-(1-methyl-pyrrolidin-2-yl)-ethyl]-amine

MS(APCI+)=544

C26CPA1% Inhibition @ 1 μM=98.5% C26CPA1% Inhibition @ 0.1 μM=53.7%

### EXAMPLE 63

### {5-[3,4-Difluoro-2-(2-fluoro-4-iodo-phenylamino)-phenyl]-[1,3,4]oxadiazol-2-yl}-(4-pyrrolidin-1-yl-butyl)-amine

MS(APCI+)=558

C26CPA1 IC₅₀=0.023 μM

C26CPA1% Inhibition @ 0.1 μM=%

### EXAMPLE 64

### N*4*-{5-[3,4-Difluoro-2-(2-fluoro-4-iodo-phenylamino)-phenyl]-[1,3,4]oxadiazol-2-yl}-N*1* ,N*1*-diethyl-pentane-1,4-diamine

MS(APCI+)=574

C26CPA1% Inhibition @ 1 μM=18.2%

C26CPA1% Inhibition @ 0.1 μM=0%

### EXAMPLE 65

### {5-[3,4-Difluoro-2-(2-fluoro-4-iodo-phenylamino)-phenyl]-[1,3,4]oxadiazol-2-yl}-(tetrahydro-furan-2-ylmethyl)-amine

MS(APCI+)=5 17

C26CPA1% Inhibition @ 1 μM=12.7%

C26CPA1% Inhibition @ 0.1 μM=0%

### EXAMPLE 66

### {5-[3,4-Difluoro-2-(2-fluoro-4-iodo-phenylamino)-phenyl]-[1,3,4]oxadiazol-2-yl}-bis-(2-methoxy-ethyl)-amine

MS(APCI+)=549

C26CPA1% Inhibition @ 1 μM=12.5%

C26CPA1% Inhibition @ 0.1 μM=0%

### EXAMPLE 67

### N-{5-[2-(4-Ethyl-2-fluoro-phenylamino)-3,4-difluoro-phenyl]-[1,3,4]oxadiazol-2-yl}-N′-methyl-ethane-1,2-diamine

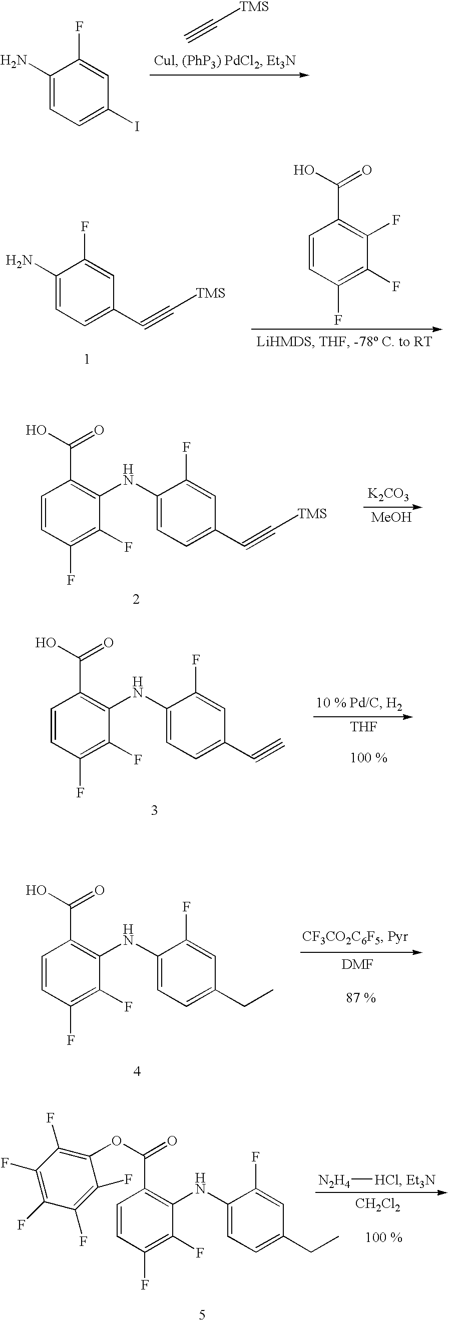

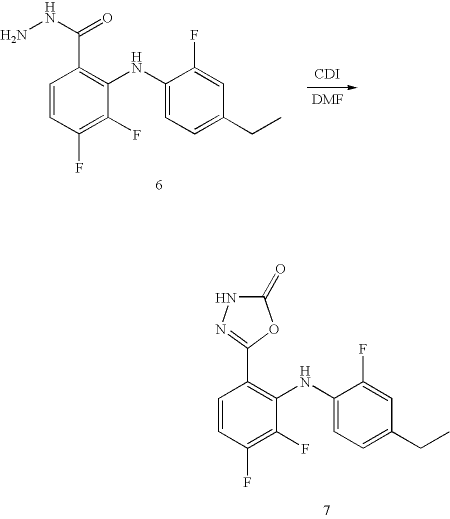

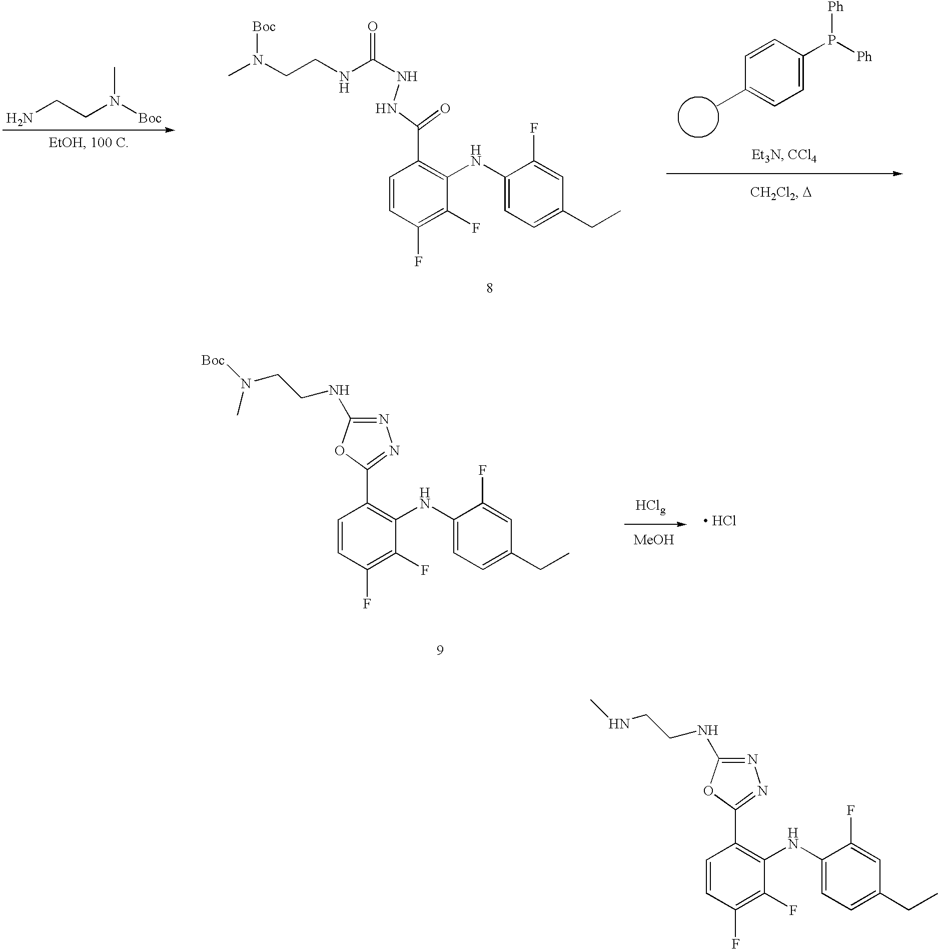

m.p.=154-157° C.; ¹NMR (400 MHz; CD₃OD) 7.57-7.61 (m, 1H), 6.87-6.99 (m, 4H), 3.68 (t, 2H, J=5.9 Hz), 3.3 (2H, under CD₃OD), 2.74 (s, 3H), 2.61 (q, 2H, J=7.6 Hz), 1.21 (t, 3H, J=7.6 Hz); MS(APCI+)=392; Anal. calcd/found for C₁₉H₂₀F₃IN₅O₁ with 2.29 moles of HCl: C 48.05/47.66, H 4.73/4.93, N 14.75/14.55, F 12.00/11.99.

C26CPA1 C₅₀=0.0160 μM

An alternative 4′ ethyl oxadiazole synthesis comprises utilizing the steps outlined above, with the use of an unprotected primary amine in the synthesis of compound 8, as depicted below:

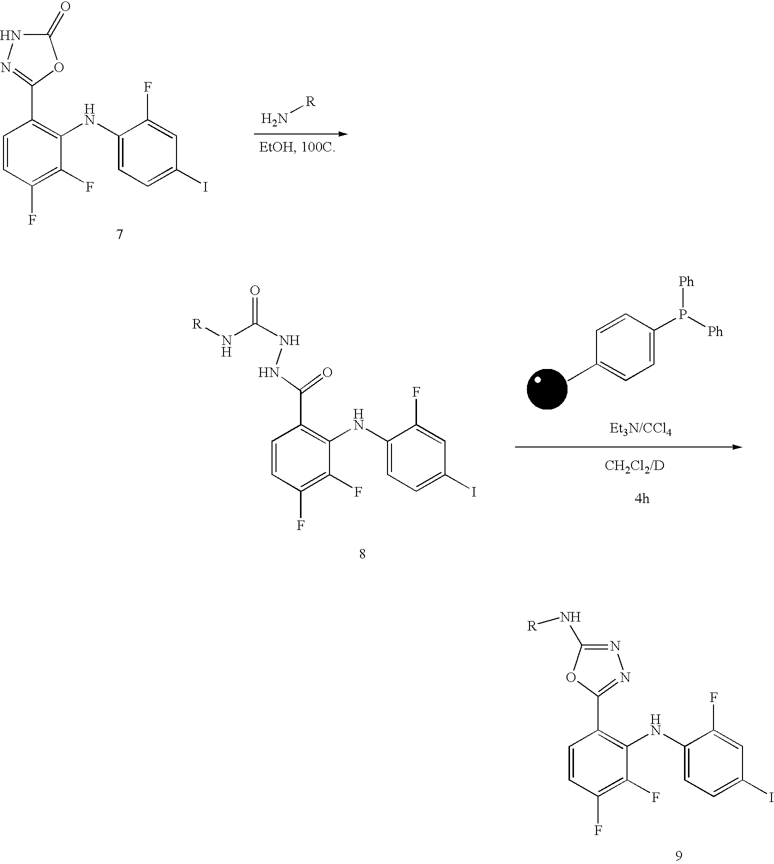

### EXAMPLE 68

### {5-[2-(4-Ethyl-2-fluoro-phenylamino)-3,4-difluoro-phenyl]-[1,3,4]oxadiazol-2-yl}-(2-morpholin-4-yl-ethyl)-amine

m.p.=233-238° C.; ¹NMR (400 MHz; DMSO-d₆) 10.86 (bs, 1H), 8.83 (bs, 1H), 8.33 (t, 1H, J=5.9 Hz), 7.52-7.56 (m, 1H), 7.15 (dd, 1H, J=16.6, 9.3 Hz), 7.07-7.10 (m, 1H), 6.91-6.98 (m, 2H), 3.91-3.94 (m, 2H), 3.73-3.79 (m, 2H), 3.64-3.69 (m, 2H), 3.46-3.49 (m, 2H), 3.32-3.37 (m, 2H), 3.07-3.10 (m, 2H), 2.54 (q, 2H, J=7.6 Hz), 1.13 (t, 3H, J=7.6 Hz); MS(APCI+)=448; Anal. calcd/found for C₁₉H₂₀F₃IN₅O₁ with 2.06 moles of HCl: C 50.57/50.20, H 5.03/4.91, N 13.40/13.06, F 10.91/10.91.

C26CPA1 IC₅₀=0.050 μM

### EXAMPLE 69

### 2-{5-[2-(4-Ethyl-2-fluoro-phenylamino)-3,4-difluoro-phenyl]-[1,3,4]oxadiazol-2-ylamino}-ethanol

The title compound is synthesized as described below:

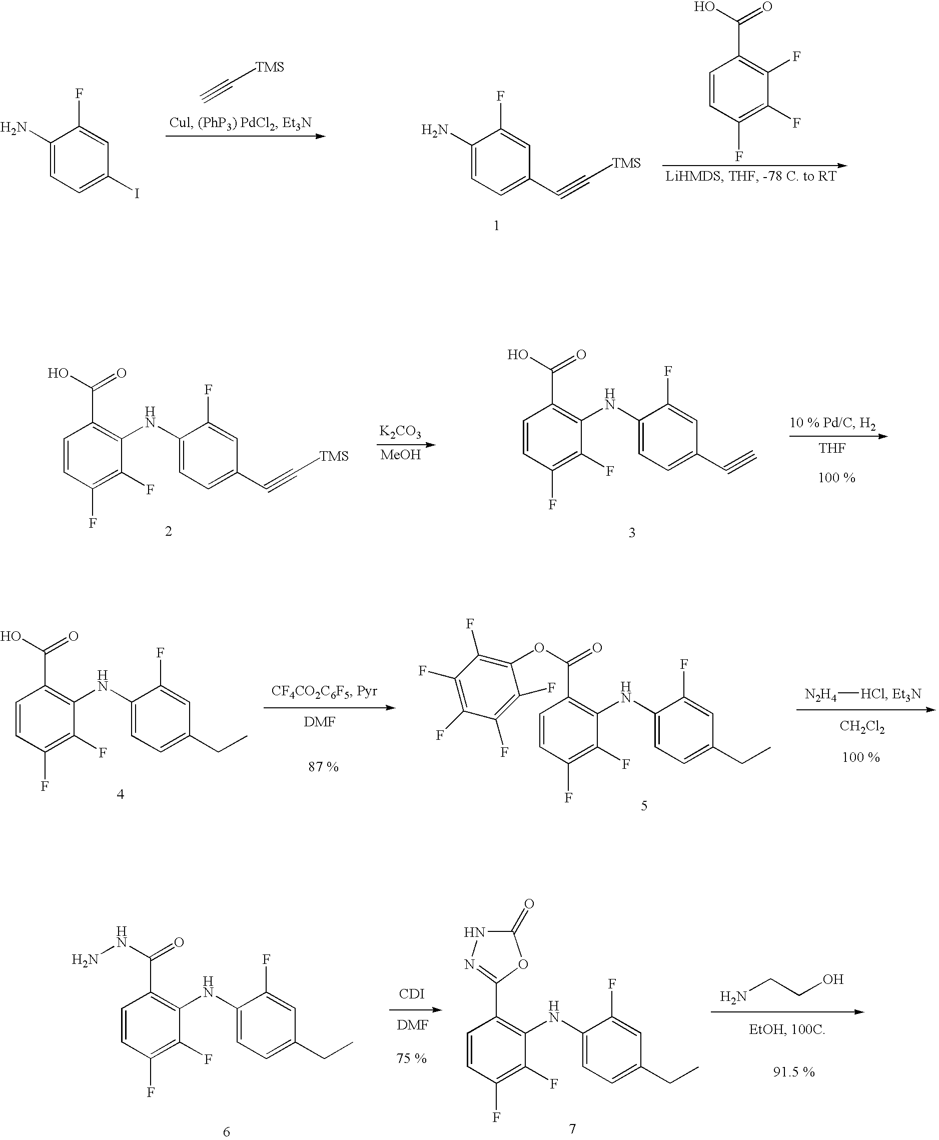

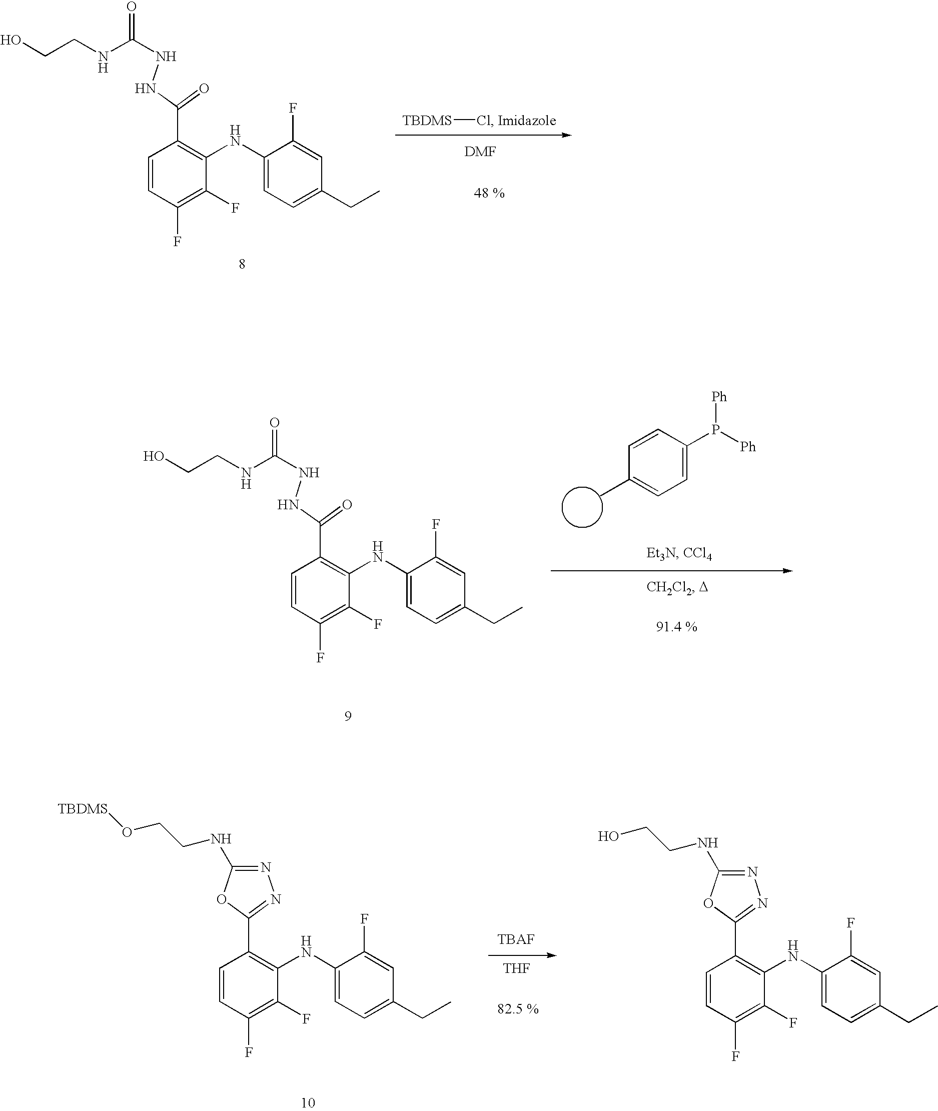

Step 1

2-Fluoro-4-iodoaniline (5.00 g, 21.1 mmol.), Cul (90 mg, 0.42 mmol) and (Ph₃P)₂PdCl₂ (300 mg, 0.42 mmol) were weighed into a flask which was sealed and flushed with nitrogen. A solution of TMS-acetylene (2.28 g, 23.2 mmol in TEA (20 mL) was added, then the entire mixture stirred 15 hours at RT. The reaction mixture was diluted with diethyl ether (200 mL), filtered through Celite, then all solvents removed under reduced pressure. The resulting dark brown oil was purified by filtration through a plug of silica (5% EtOAc/hexanes) to afford 2-fluoro-4[(trimethylsilyl)ethynyl]aniline as a pale brown oil which rapidly solidified to give a crystalline solid (3.85 g, 88%); mp (EtOAc/hexanes) 45-47° C.

Step 2

A mixture of 2-fluoro-4[(trimethylsilyl)ethynyl]aniline (3.85 g, 18.6 mmol) and 2,3,4-trifluorobenzoic acid (3.27 g, 18.6 mmol) was dissolved in dry THF (25 mL). The flask was fitted with a pressure-equalizing dropping funnel and the entire apparatus evacuated and flushed with nitrogen. The solution was then cooled to −78° C. and a solution of 1.06M LiHMDS (52.64 mL, 55.8 mmol) was added dropwise from the dropping funnel. Following this addition, the reaction mixture was allowed to warm to room temperature and stirred for a further 15 hours. The reaction solvent was removed under reduced pressure and the resulting residue partitioned between 1 M HCl (100 mL) and EtOAc (2×100 mL). The combined EtOAc fractions were then washed with water (100 mL) and brine (100 mL), dried over Na₂SO₄, and the EtOAc. removed under reduced pressure to afford a crude product which was purified by chromatography on flash silica (10% EtOAc/hexanes), affording 3,4-difluoro-2-{[2-fluoro-4(trimethylsilylethynyl)phenyl]amino} benzoic acid as a pale yellow solid (3.99 g, 59%); m.; (EtoAc/hexanes) 164-167° C.

Step 3

3,4-difluoro-2-{[2-fluoro-4(trimethylsilylethynyl)phenyl]amino} benzoic acid (3.99 g, 11.0 mmol) was dissolved in methanol (200 mL) to which was added K₂CO₃ (3.03 g, 22.0 mmol). This mixture was stirred at RT for 15 hours, then the reaction solvent removed under reduced pressure. The resulting residue was dissolved in water (50 mL), to which was added 1 M HCl until the pH=4. The resulting pale brown precipitate was collected by filtration and dried to afford 3,4-difluoro[(4-ethynyl-2-fluorophenyl)amino]benzoic acid (3.17 g, 99%); m.p. (EtoAc/hexanes) 160-162° C.

Step 4

To an Ace Glass pressure reaction vessel was added 2-(4-ethynyl-2-fluoro-phenylamino)-3,4-difluoro-benzoic acid (13.40 g, 46.01 mmol), 0.50 g of 10% Palladium (dry, unreduced) on Carbon and 100 mL tetrahydrofuran. The vessel was placed on a Parr shaking apparatus. The vessel was flushed three times with nitrogen followed by flushing five times with hydrogen. Following the flushing sequence the vessel was then pressurized to 50 psi with hydrogen. The reaction was than shaken for 18.8 hours then checked for completeness. Upon notification of the reaction being complete it was then filtered through a medium fritted filter funnel and returned to the chemist. The reaction mixture was concentrated in vacuo and the affording yellow-orange oil was partitioned between ethyl acetate and 0.1 M HCl aqueous solution. Organics were washed several times with 0.1 M HCl and twice with brine then collected, dried over Na₂SO₄, filtered and concentrated in vacuo to afford 2-(4-ethyl-2-fluoro-phenylamino)-3,4-difluoro-benzoic acid as a yellow solid (13.92 g, >100% yield due to remaining solvent).

Step 5

To a stirring solution of 2-(4-ethyl-2-fluoro-phenylamino)-3,4-difluoro-benzoic acid (3.06 g, 10.36 mmol) in DMF (25 mL) is added pyridine (0.92 mL, 11.40 mmol) and pentafluorophenyltrifluoro acetate (1.96 mL, 11.40 mmol) and allowed to stir at ambient temperature. After stirring for two hours, the reaction mixture was partitioned between ethyl acetate and water. The organic layer was washed with saturated NaHCO₃, twice with 1.0 M HCl solution and twice with brine. Organics were collected, dried over Na₂SO₄, filtered and concentrated in vacuo. The afforded orange oil was filtered through a plug of silica gel in 4:1 hexanes/ethyl acetate affording 2-(4-ethyl-2-fluoro-phenylamino)-3,4-difluoro-benzoic acid pentafluorophenyl ester 5 as an orange oil (4.17 g, 87.3%).

Step 6

To a stirring solution of hydrazine hydrochloride (0.68 g, 9.87 mmol) in dichloromethane (50 mL) was added triethylamine (2.63 mL, 18.84 mmol) and allowed to stir for 30 minutes. 2-(4-ethyl-2-fluoro-phenylamino)-3,4-difluoro-benzoic acid pentafluorophenyl ester (4.14 g, 8.79 mmol) was added and the mixture was allowed to stir an additional 5 hours. The reaction mixture was partitioned between dichloromethane and water. Organics were washed twice with water, twice with brine, twice with saturated NaHCO₃ and a final wash with brine. Organics were collected, dried over Na₂SO₄, filtered and concentrated in vacuo. Afforded 2-(4-Ethyl-2-fluoro-phenylamino)-3,4-difluoro-benzoic acid hydrazide 6 (3.07 g, >100% yield due to solvents remaining) as a yellow solid.

Step 7

To a stirring solution of 2-(4-Ethyl-2-fluoro-phenylamino)-3,4-difluoro-benzoic acid hydrazide (2.88 g, 9.137 mmol) in DMF (40 mL) was added carbonyldiimidazole (1.58 g, 9.783 mmol) and allowed to stir at ambient temperature. After 3 hours the reaction was partitioned between ethyl acetate and water. Organics were washed twice with water and twice with brine. Organics were collected, dried over Na₂SO₄, filtered and concentrated in vacuo. Afforded 5-[2-(4-ethyl-2-fluoro-phenylamino)-3,4-difluoro-phenyl]-3H-[1,3,4]oxadiazol-2-one (2.34 g, 74.9%) as a light yellow solid.

Step 8

To a stirring solution of 5-[2-(4-ethyl-2-fluoro-phenylamino)-3,4-difluoro-phenyl]-3H-[1,3,4]oxadiazol-2-one (0.503 g, 1.500 mmol) in ethanol (10 mL) was added ethanolamine (0.27 mL, 4.500 mmol) and heated to 100° C. After refluxing for 16 hours, the reaction mixture was partitioned between ethyl acetate and water. Organics were washed twice with saturated NaHCO₃ and twice with brine. Organics were collected, dried over Na₂SO₄, filtered and concentrated in vacuo to afford 4′-(2-amino-ethanol)-1′-[3,4-difluoro-2-(2-fluoro-4-ethyl-phenylamino)]-semicarbazole 8 as a light yellow solid (0.54 g, 91.5%).

Step 9

To a stirring solution of 4′-(2-amino-ethanol)-1′-[3,4-difluoro-2-(2-fluoro-4-ethyl-phenylamino)]-semicarbazole (0.54 g, 1.362 mmol) in DMF (10 mL) was added imidazole (0.090 g, 1.499 mmol) and tert-butyldimethylsilyl chloride (0.230 g, 1.499 mmol) and allowed to stir at ambient temperature. After stirring for three hours, the reaction mixture was partitioned between ethyl acetate and water. Organics were washed twice with 0.1 M HCl solution, twice with water and twice with brine. Organics were collected, dried over Na₂SO₄, filtered and concentrated in vacuo to afford a yellow glass (0.61 g). The yellow glass was purified via silica column chromatography in 2:1 hexanes/ethyl acetate to 1:1 hexanes/ethyl acetate to afford 4′-(2-(tert-butyl-dimethyl-silanyloxy)-ethylamine)-1′-[3,4-difluoro-2-(2-fluoro-4-ethyl-phenylamino)]-semicarbazole 9 (0.334 g, 48.1%) as a white foam.

Step 10

To a stirring solution of 4′-(2-(tert-butyl-dimethyl-silanyloxy)-ethylamine)-1′-[3,4-difluoro-2-(2-fluoro-4-ethyl-phenylamino)]-semicarbazole (0.33 g, 0.65 mmol) in dichloromethane (15 mL) was added resin bound triphenylphosphine (0.82 g, 1.29 mmol), triethylamine (0.72 mL, 5.17 mmol), and carbontetrachloride (0.16 mL, 1.94 mmol) and heated to reflux (oil bath temperature was set to 46° C.). After refluxing for three hours the reaction was filtered and the resin was rinsed several times with 5% methanol in dichloromethane. The filtrate was collected and concentrated in vacuo. The affording residue was partitioned between ethyl acetate and water. Organics were washed twice with water and twice with brine. Organics were collected, dried over Na₂SO₄, filtered and concentrated in vacuo to afford [2-(tert-butyl-dimethyl-silanyloxy)-ethyl]-{5-[2-(4ethyl-2-fluoro-phenylamino)-3,4-difluoro-phenyl]-[1,3,4]oxadiazol-2-yl}-amine 10 as a yellow foam (0.288 g, 90.4%).

Step 11

To a stirring solution of [2-(tert-butyl-dimethyl-silanyloxy)-ethyl]-{5-[2-(4ethyl-2-fluoro-phenylamino)-3,4-difluoro-phenyl]-[1,3,4]oxadiazol-2-yl}-amine (0.28 g, 0.57 mmol) in tetrahydrofuran at 0° C. was added acetic acid (0.033 mL, 0.57 mmol) and tetrabutylammonium fluoride solution in THF (1.0 M, 0.85 ml, 0.85 mmol). The ice bath was removed and the reaction was allowed to warm to ambient temperature and stirred for 4 hours. The reaction mixture was partitioned between ethyl acetate and water and the organic layer was washed twice with saturated NaHCO₃ and once with 1.0 M HCl solution. Organics were washed twice with brine then collected, dried over Na₂SO₄, filtered and concentrated in vacuo. The affording yellow foam was crystallized from dichloromethane and hexanes to afford 2-{5-[2-(4-ethyl-2-fluoro-phenylamino)-3,4-difluoro-phenyl]-[1,3,4]oxadiazol-2-ylamino}-ethanol (0.177 g, 82.5%) as a white solid. m.p.=100-103° C.; ¹NMR (400 MHz; CD₃OD) 7.55-7.59 (m, 1H), 6.87-6.98 (m, 4H), 3.71 (t, 2H, J=5.6 Hz), 3.42 (t, 2H, J=5.6 Hz), 2.61 (q, 2H, J=7.8 Hz), 1.21 (t, 3H, J=7.8 Hz); MS(APCI+)=379; Anal. calcd/found for C₁₈H₁₇F₃IN₄O₂: C 57.14/56.97, H 4.53/4.43, N 14.81/14.59, F 15.06/14.70. C26CPA 1 IC₅₀=0.100 μM

### EXAMPLE 70

### Allyl-{5-[2-(4-ethyl-2-fluoro-phenylamino)-3,4-difluoro-phenyl]-[1,3,4]oxadiazol-2-yl}-amine

m.p.=120-123° C.; ¹NMR (400 MHz; CD₃OD) 7.54-7.58 (m, 1H), 6.87-6.98 (m, 4H), 5.89-5.99 (m, 1H), 5.29 (ddt, 1H, J=17, 3.2, 1.5 Hz), 5.17 (ddt, 1H, J=10.3, 2.9, 1.5 Hz), 3.92-3.94 (m, 2H), 2.60 (q, 2H, J=15.1, 7.6), 1.21 (t, 3H, J=7.6 Hz); MS(APCI+)=375; Anal. calcd/found for C₁₈H₁₇F₃IN₄O₂ with 0.01 moles of residual CH₂Cl₂: C 60.85/60.54, H 4.57/4.34, N 14.93/14.54, F 15.19/14.70. C26CPA1 IC₅₀=μM

### EXAMPLE 71

### {5-[3,4-Difluoro-2-(2-fluoro-4-iodo-phenylamino)-phenyl]-[1,3,4]thiadiazol-2-yl}-ethyl-amine

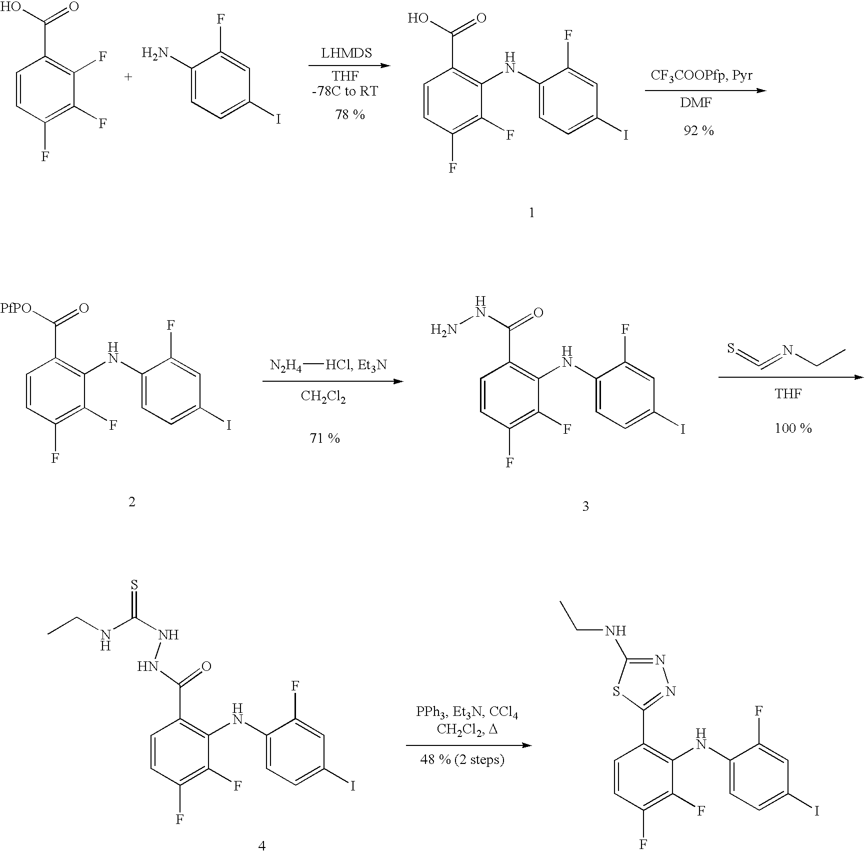

m.p.=238-239° C.; ¹NMR (400 MHz; DMSO-d₆) 8.64 (s, 1H), 7.77-7.86 (m, 2H), 7.56 (dd, 1H, J=11.0, 1.5 Hz), 7.39 (dt, 1H, J=17.6, 9.5 Hz), 7.26 (d, 1H, J=8.3 Hz), 6.34-6.38 (m, 1H), 3.2-3.3 (2H, under HDO), 1.12 (t, 3H, J=7.1);

MS(APCI+)=477; Anal. calcd/found for C₁₆H₁₂F₃IN₄S₁: C 40.35/40.28, H 2.54/2.32, N 11.76/11.54, F 11.97/12.05.

C26CPA1 IC₅₀=0.180 μM

### EXAMPLE 72

### {5-[2-(4-Ethyl-2-fluoro-phenylamino)-3,4-difluoro-phenyl]-1,3,4-oxadiazol-2-yl}-(3-pyrrolidin-1-yl-propyl)-amine

MS(APCI+)=446

### EXAMPLE 73

### N¹,N¹-Diethyl-N2-{5-[2-(4-ethyl-2-fluoro-phenylamino)-3,4-difluoro-phenyl]-1,3,4-oxadiazol-2-yl}-ethane-1,2-diamine

### EXAMPLE 74

### {5-[2-(4-Ethyl-2-fluoro-phenylamino)-3,4-difluoro-phenyl]-1,3,4-oxadiazol-2-yl}-isopropyl-amine

### EXAMPLE 75

### [2,3-Difluoro-6-(5-pyrrolidin-1-yl-1,3,4-oxadiazol-2-yl)-phenyl]-(4-ethyl-2-fluoro-phenyl)-amine

MS(APCI+)=389

C26CPA1% Inhibition @ 1 μM=39.1%

C26CPA1% Inhibition @ 1 μM=30.6%

### EXAMPLE 76

### N¹-{5-[2-(4-Ethyl-2-fluoro-phenylamino)-3,4-difluoro-phenyl]-1,3,4-oxadiazol-2-yl}-N>3-methyl-propane-1,3-diamine

### EXAMPLE 77

### {5-[2-(4-Ethyl-2-fluoro-phenylamino)-3,4-difluoro-phenyl]-1,3,4-oxadiazol-2-yl}-furan-2-ylmethyl-amine

### EXAMPLE 78

### (2,2-Dimethoxy-ethyl-{5-[2-(ethyl-2-fluoro-phenylamino)-3,4-difluoro-phenyl]-1,3,4-oxadiazol-2-yl}-amine

MS(APCI+)=423

### EXAMPLE 79

### {2,3-Difluoro-6-[5-(4-phenyl-Piperazin-1-yl)-1,3,4-oxadiazol-2-yl]-phenyl}-(4-ethyl-2-fluoro-phenyl)-amine

### EXAMPLE 80

### {5-[2-(4-Ethyl-2-fluoro-phenylamino)-3,4-difluoro-phenyl]-1,3,4-oxadiazol-2-yl}-(2-methoxy-ethyl)-amine

### EXAMPLE 81

### 6-{5-[2-(4-Ethyl-2-fluoro-phenylamino)-3,4-difluoro-phenyl]-1,3,4-oxadiazol-2-ylamino}-hexanenitrile

### EXAMPLE 82

### (3,3-Diethoxy-propyl)-{5-[2-(4-ethyl-2-fluoro-phenylamino)-3,4-difluoro-phenyl]-1,3,4-oxadiazol-2-yl}-amine

### EXAMPLE 83

### Diallyl-{5-[2-(4-ethyl-2-fluoro-phenylamino)-3,4-difluoro-phenyl]-1,3,4-oxadiazol-2-yl}-amine

MS(APCI+)=415

C26CPA1% Inhibition @ 1 μM=77.9%

C26CPA1% Inhibition @ 0.1 μM=65.6%

### EXAMPLE 84

### {5-[2-(4-Ethyl-2-fluoro-phenylamino)-3,4-difluoro-phenyl]-1,3,4-oxadiazol-2-yl}-(3-methylsulfanyl-propyl)-amine

### EXAMPLE 85

### 4-{5-[2-(4-Ethyl-2-fluoro-phenylamino)-3,4-difluoro-phenyl]-1,3,4-oxadiazol-2-ylamino}-isoxazolidin-3-one

### EXAMPLE 86

### N¹-Ethyl-N¹-{5-[2-(4-ethyl-2-fluoro-phenylamino)-3,4-difluoro-phenyl]-1,3,4-oxadiazol-2-yl}-N²,N²-dimethyl-ethane-1,2-diamine

MS(APCI+)=434

### EXAMPLE 87

### {2,3-Difluoro-6-[5-(4-methyl-perhydro-1,4-diazepin-1-yl)-1,3,4-oxadiazol-2-yl]-phenyl }-(4-ethyl-2-fluoro-phenyl)-amine

### EXAMPLE 88

### 1-{5-[2-(4-Ethyl-2-fluoro-phenylamino)-3,4-difluoro-phenyl]-1,3,4-oxadiazol-2-yl}-piperidine-3-carboxylic Acid Diethylamide

### EXAMPLE 89

### N-{5-[2-(4-Ethyl-2-fluoro-phenylamino)-3,4-difluoro-phenyl]-1,3,4-oxadiazol-2-yl}-O-(tetrahydro-pyran-2-yl)-hydroxylamine

### EXAMPLE 90

### {5-[2-(4-Ethyl-2-fluoro-phenylamino)-3,4-difluoro-phenyl]-1,3,4-oxadiazol-2-yl}-(3-morpholin-4-yl-propyl)-amine

### EXAMPLE 91

### {5-[2-(4-Ethyl-2-fluoro-phenylamino)-3,4-difluoro-phenyl]-1,3,4-oxadiazol-2-yl}-(4H-1,2,4-triazol-3-yl)-amine

### EXAMPLE 92

### N¹-{5-[2-(4-Ethyl-2-fluoro-phenylamino)-3,4-difluoro-phenyl]-1,3,4-oxadiazol-2-yl}-N2-methyl-ethane-1,2-diamine

### EXAMPLE 93

### N¹-{5-[2-(4-Ethyl-2-fluoro-phenylamino)-3,4-difluoro-phenyl]-1,3,4-oxadiazol-2-yl}-N1,N>3,N>3-trimethyl-propane-1.3-diamine

MS(APCI+)=434

C26CPA1% Inhibition @ 1 μm=31.9%

C26CPA1% Inhibition @ 0.1 μM=0%

### EXAMPLE 94

### {2,3-Difluoro-6-[5-(4-pyridin-2-yl-piperazin-1-yl)-1,3,4-oxadiazol-2-yl]-phenyl}-(4-ethyl-2-fluoro-phenyl)-amine

### EXAMPLE 95

### N²-{5-[2-(4-Ethyl-2-fluoro-phenylamino)-3,4-difluoro-phenyl]-1,3,4-oxadiazol-2-yl}-N1N1-dimethyl-propane-1,2-diamine

### EXAMPLE 96

### N¹-(3-Dimethylamino-propyl)-N¹-{5-[2-(4-ethyl-2-fluoro-phenylamino)-3,4-difluoro-phenyl]-1,3,4-oxadiazol-2-yl}-N>3,N>3-dimethyl-propane-1,3-diamine

MS(APCI+)=505

C26CPA1% Inhibition @ 1 μM=55%

C26CPA1% Inhibition @ 0.1 μM=54%

### EXAMPLE 97

### N¹-Cyclohexyl-N>3-{5-[2-(4-ethyl-2-fluoro-phenylamino)-3,4-difluoro-phenyl]-1,3,4-oxadiazol-2-yl}-propane-1,3-diamine

### EXAMPLE 98

### {5-[2-(4-Ethyl-2-fluoro-phenylamino)-3,4-difluoro-phenyl]-1,3,4-oxadiazol-2-yl}-(2-phenoxy-ethyl)-amine

### EXAMPLE 99

### {5-[2-(4-Ethyl-2-fluoro-phenylamino)-3,4-difluoro-phenyl]-1,3,4-oxadiazol-2-yl-(2-morpholin-4-yl-ethyl)-amine

### EXAMPLE 100

### Cyclopropylmethyl-{5-[2-(4-ethyl-2-fluoro-phenylamino)-3,4-difluoro-phenyl]-1,3,4-oxadiazol-2-yl}-propyl-amine

MS(APCI+)=431

C26CPA1% Inhibition@1 μM=40.5%

C26CPA1% Inhibition@0.1 μM=37.1%

### EXAMPLE 101

### (4-Ethyl-2-fluoro-phenyl)-{6-[5-(4-ethyl-piperazin-1-yl)-1,3,4-oxadiazol-2-yl]-2,3-difluoro-phenyl}-amine

MS(APCI+)=432

C26CPA1% Inhibition@1 μM=47.1%

C26CPA1% Inhibition@0.1 μM=41.2%

### EXAMPLE 102

### {5-[2-(4-Ethyl-2-fluoro-phenylamino)-3,4-difluoro-phenyl]-1,3,4-oxadiazol-2-yl}-methyl-(1-methyl-pyrrolidin-3-yl)-amine

MS(APCI+)=432

C26CPA1% Inhibition@1 μM=54.6%

C26CPA1% Inhibition@0.1 μM=42.4%

### EXAMPLE 103

### N¹,N¹-Diethyl-N2-(2-{5-[2-(4-ethyl-2-fluoro-phenylamino)-3,4-difluoro-phenyl]-1,3,4-oxadiazol-2-ylamino}-ethyl)-ethane-1,2-diamine

### EXAMPLE 104

### {5-[2-(4-Ethyl-2-fluoro-phenylamino)-3,4-difluoro-phenyl]-1,3,4-oxadiazol-2-yl}-thiophen-2-ylmethyl-amine

### EXAMPLE 105

### {2,3-Difluoro-6-[5-(4-pyrrolidin-1-yl-piperidin-1-yl)-1,3,4-oxadiazol-2-yl]-phenyl}-(4-ethyl-2-fluoro-phenyl)-amine

### EXAMPLE 106

### {5-[2-(4-Ethyl-2-fluoro-phenylamino)-3,4-difluoro-phenyl]-1,3,4-oxadiazol-2-yl}-(tetrahydro-pyran-2-ylmethyl)-amine

### EXAMPLE 107

### {5-[2-(4-Ethyl-2-fluoro-phenylamino)-3,4-difluoro-phenyl]-1,3,4-oxadiazol-2-yl}-(2-pyridin-4-yl-ethyl)-amine

### EXAMPLE 108

### {6-[5-(4-Benzyl-perhydro-1,4-diazepin-1-yl)-1,3,4-oxadiazol-2-yl]-2,3-difluoro-phenyl}-(4-ethyl-2-fluoro-phenyl)-amine

### EXAMPLE 109

### {5-[2-(4-Ethyl-2-fluoro-phenylamino)-3,4-difluoro-phenyl]-1,3,4-oxadiazol-2-yl}-pyridin-2-ylmethyl-amine

### EXAMPLE 110

### {5-[2-(4-Ethyl-2-fluoro-phenylamino)-3,4-difluoro-phenyl]-1,3,4-oxadiazol-2-yl}-(5-methyl-furan-2-ylmethyl)-amine

### EXAMPLE 111

### (2-Ethoxy-benzyl)-{5-[2-(4-ethyl-2-fluoro-phenylamino)-3,4-difluoro-phenyl]-1,3,4-oxadiazol-2-yl}-amine

### EXAMPLE 112

### {2,3-Difluoro-6-[5-(4-methyl-piperazin-1-yl)-1,3,4-oxadiazol-2-yl]-phenyl}-(4-ethyl-2-fluoro-phenyl)-amine

MS(APCI+)=418

C26CPA1% Inhibition@1 μM=84%

C26CPA1% Inhibition@0.1 μM=62.6%

### EXAMPLE 113

### {5-[2-(4-Ethyl-2-fluoro-phenylamino)-3,4-difluoro-phenyl]-1,3,4-oxadiazol-2-yl}-pyridin-3-ylmethyl-amine

### EXAMPLE 114

### {5-[2-(4-Ethyl-2-fluoro-phenylamino)-3,4-difluoro-phenyl]-1,3,4-oxadiazol-2-yl}-(2-thiophen-2-yl-ethyl)-amine

MS(APCI+)=445

C26CPA1% Inhibition@1 μM=46.3%

C26CPA1% Inhibition@0.1 μM=35.4%

### EXAMPLE 115

### {5-[2-(4-Ethyl-2-fluoro-phenylamino)-3,4-difluoro-phenyl]-1,3,4-oxadiazol-2-yl}-(2-pyridin-3-yl-ethyl)-amine

### EXAMPLE 116

### {5-[2-(4-Ethyl-2-fluoro-phenylamino)-3,4-difluoro-phenyl]-1,3,4-oxadiazol-2-yl}-pyridin-4-ylmethyl-amine

### EXAMPLE 117

### {5-[2-(4-Ethyl-2-fluoro-phenylamino)-3,4-difluoro-phenyl]-1,3,4-oxadiazol-2-yl}-(2-pyridin-2-yl-ethyl)-amine

### EXAMPLE 118

### N-(2-{5-[2-(4-Ethyl-2-fluoro-phenylamino)-3,4-difluoro-phenyl]-1,3,4-oxadiazol-2-ylamino}-ethyl)-acetamide

### EXAMPLE 119

### {5-[2-(4-Ethyl-2-fluoro-phenylamino)-3,4-difluoro-phenyl]-1,3,4-oxadiazol-2-yl}-(6-methoxy-pyridin-3-yl)-amine

### EXAMPLE 120

### {2,3-Difluoro-6-[5-(2-pyridin-4-yl-pyrrolidin-1-yl)-1,3,4-oxadiazol-2-yl]-phenyl}-(4-ethyl-2-fluoro-phenyl)-amine

### EXAMPLE 121

### {5-[2-(4-Ethyl-2-fluoro-phenylamino)-3,4-difluoro-phenyl]-1,3,4-oxadiazol-2-yl}-(2-methanesulfonyl-ethyl)-methyl-amine

MS(APCI+)=455

C26CPA1% Inhibition@1 μM=26.9%

C26CPA1% Inhibition@0.1 μM=0%

### EXAMPLE 122

### N²-{5-[2-(4-Ethyl-2-fluoro-phenylamino)-3,4-difluoro-phenyl]-1,3,4-oxadiazol-2-yl}-N¹,N¹-dimethyl-1-phenyl-ethane-1,2-diamine

### EXAMPLE 123

### {2,3-Difluoro-6-[5-(4-pyridin-2-ylmethyl-piperazin-1-yl)-1,3,4-oxadiazol-2-yl]-phenyl}-(4-ethyl-2-fluoro-phenyl)-amine

MS(APCI+)=495

C26CPA1% Inhibition@1 μM=0%

C26CPA1% Inhibition@0.1 μM=0%

### EXAMPLE 124

### {5-[2-(4-Ethyl-2-fluoro-phenylamino)-3,4-difluoro-phenyl]-1,3,4-oxadiazol-2-yl}-(2-morpholin-4-yl-1-phenyl-ethyl)-amine

### EXAMPLE 125

### {2,3-Difluoro-6-[5-(3-pyridin-4-yl-pyrrolidin-1-yl)-1,3,4-oxadiazol-2-yl]-phenyl}-(4-ethyl-2-fluoro-phenyl)-amine

### EXAMPLE 126

### {5-[2-(4-Ethyl-2-fluoro-phenylamino)-3,4-difluoro-phenyl]-1,3,4-oxadiazol-2-yl}-methyl-(2-pyridin-4-yl-ethyl)-amine

### EXAMPLE 127

### {5-[2-(4-Ethyl-2-fluoro-phenylamino)-3,4-difluoro-phenyl]-1,3,4-oxadiazol-2-yl}-methyl-(2-pyridin-2-yl-ethyl)-amine

MS(APCI+)=454

C26CPA1% Inhibition@1 μM=0%

C26CPA1% Inhibition@0.1 μM=0%

### EXAMPLE 128

### {5-[2-(4-Ethyl-2-fluoro-phenylamino)-3,4-difluoro-phenyl]-1,3,4-oxadiazol-2-yl}-(2-morpholin-4-yl-2-pyridin-3-yl-ethyl)-amine

MS(APCI+)=525

C26CPA1% Inhibition@1 μM=0%

C26CPA1% Inhibition@0.1 μM=0%

### EXAMPLE 129

### N¹-{5-[2-(4-Ethyl-2-fluoro-phenylamino)-3,4-difluoro-phenyl]-1,3,4-oxadiazol-2-yl}-N²,N²-dimethyl-1-phenyl-ethane-1,2-diamine

### EXAMPLE 130

### {5-[2-(4-Ethyl-2-fluoro-phenylamino)-3,4-difluoro-phenyl]-1,3,4-oxadiazol-2-yl}-(3-imidazol-1-yl-propyl)-amine

MS(APCI+)=443

### EXAMPLE 131

### {5-[2-(4-Ethyl-2-fluoro-phenylamino)-3,4-difluoro-phenyl]-1,3,4-oxadiazol-2-yl}-methyl-(tetrahydro-pyran-2-ylmethyl)-amine

### EXAMPLE 132

### {5-[2-(4-Ethyl-2-fluoro-phenylamino)-3,4-difluoro-phenyl]-1,3,4-oxadiazol-2-yl}-(2-furan-2-yl-ethyl)-amine

MS(APCI+)=429

### EXAMPLE 133

### {5-[2-(4-Ethyl-2-fluoro-phenylamino)-3,4-difluoro-phenyl]-1,3,4-oxadiazol-2-yl}-(2-thiophen-2-yl-thiazol-4-ylmethyl)-amine

### EXAMPLE 134

### {5-[2-(4-Ethyl-2-fluoro-phenylamino)-3,4-difluoro-phenyl]-1,3,4-oxadiazol-2-yl}-(5-methyl-isoxazol-3-ylmethyl)-amine

### EXAMPLE 135

### {5-[2-(4-Ethyl-2-fluoro-phenylamino)-3,4-difluoro-phenyl]-1,3,4-oxadiazol-2-yl}-[2-(4-methyl-1H-imidazol-2-yl)-ethyl]-amine

### EXAMPLE 136

### {5-[2-(4-Ethyl-2-fluoro-phenylamino)-3,4-difluoro-phenyl]-1,3,4-oxadiazol-2-yl }-[2-(5-pyridin-4-yl-2H-1,2,4-triazol-3-yl)-ethyl]-amine

### EXAMPLE 137

### {5-[2-(4-Ethyl-2-fluoro-phenylamino)-3,4-difluoro-phenyl]-1,3,4-oxadiazol-2-yl}-(1-methyl-2-thiophen-3-yl-ethyl)-amine

MS(APCI+)=459

C26CPA1% Inhibition@1 μM=85.3%

C26CPA1% Inhibition@0.1 μM=72.1%

### EXAMPLE 138

### {5-[2-(4-Ethyl-2-fluoro-phenylamino)-3,4-difluoro-phenyl]-1,3,4-oxadiazol-2-yl}-(1H-tetrazol-5-ylmethyl)-amine

### EXAMPLE 139

### {5-[2-(4-Ethyl-2-fluoro-phenylamino)-3,4-difluoro-phenyl]-1,3,4-oxadiazol-2-yl}-(3-phenyl-1H-pyrazol-4-ylmethyl)-amine

### EXAMPLE 140—METHOD A

### 2-{5-[2-(4-ethynyl-2-fluoro-phenylamino)-3,4-difluoro-phenyl]-[1,3,4]oxadiazol-2-ylamino}-ethanol

To a stirring solution comprised of 2-{5-[3,4-difluoro-2-(2-fluoro-4-iodo-phenylamino)-phenyl]-[1,3,4]oxadiazol-2-ylamino}-ethanol (0.330 g, 0.693 mmole), cuprous iodide (0.02 g, 0.1 mmole), dichlorobis(triphenylphosphine)-palladium(II) (0.029 g, 0.041 mmole), and triethylamine (0.25 mL, 1.8 mmole) in tetrahydrofuran (10 mL) was added ethynyl-trimethylsilane (0.12 mL, 0.85 mmole). The reaction mixture was stirred for two hours at ambient temperature. After two hours, a second portion 0.12 mL portion of ethylnyl-trimethylsilane was added, and the reaction was stirred for an additional thirty minutes. The mixture was partitioned between very dilute aqueous hydrochloric acid and ether. The ether phase was dried (MgSO₄) and concentrated to 0.6 g of a sticky semisolid that was purified by flash chromatography. Elution with a gradient (100% dichloromethane to 5% methanol over 36 minutes) removed solvent-front impurities. The isolated material was carried on directly to the next step. The purified intermediate 2-{5-[3,4-difluoro-2-(2-fluoro-4-trimethylsilanylethynyl-phenylamino)-phenyl]-[1,3,4]oxadiazol-2-ylamino}1-ethanol was dissolved in methanol (20 mL) and to the solution was added cesium fluoride (5 g, 33 mmole) and the reaction mixture was stirred for fifteen hours at ambient temperature under a nitrogen atmosphere. The mixture was concentrated in vacuo to a crude solid that was partitioned between water and ether. The ether layer was died (MgSO₄) and concentrated in vacuo to a foam. Crystallization from chloroform afforded 0.17 g (65.4% yield over two steps) of the pale yellow globular crystalline solid product; mp 112-115° C.; ¹H-NMR (400 MHz; DMSO) δ 8.83 (s, 1H), 7.94 (t, 1H, J=5.9 Hz), 7.52 (m, 1H), 7.34 (dd, 1H, J=12.0, 1.7 Hz), 7.27 (m, 1H), 7.14 (dd, 1H, J=8.4, 1.5 Hz), 6.86 (m, 1H), 4.73 (t, 1H, J=5.6 Hz), 4.11 (s, 1H), 3.48 (dt, 2H, J=5.9, 5.6 Hz), 3.22 (dt, 2H, J=5.9, 5.9 Hz); MS (APCI+) 375.1 (M+1, 100); (APCI−) 373.1 (M-1, 61), 271.1 (100); IR 3302, 3198, 2107, 1615, 1504, 1480 cm⁻¹; % C (calculated for C₁₈H₁₃F₃N₄O₂ with 0.18 mole equiv. of CHCl₃/found) 55.17/54.84, % H 3.36/2.98, % N 14.16/13.86, % F 14.40/14.00.

### EXAMPLE 140—METHOD B

### 2-{5-[2-(4-Ethynyl-2-fluoro-phenylamino)-3,4-difluoro-phenyl]-[1,3,4]oxadiazol-2-ylamino}-ethanol

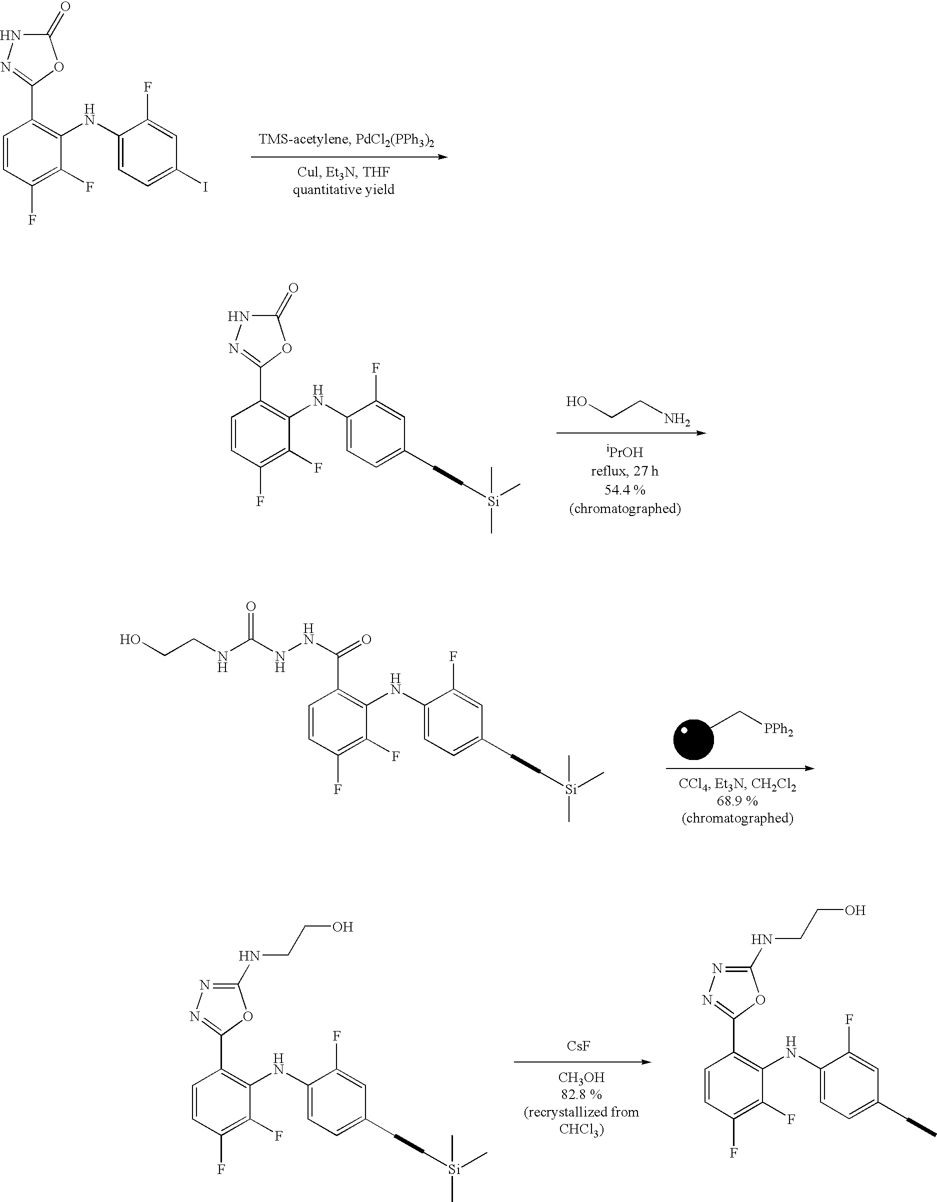

Step 1

5-[3,4-Difluoro-2-(2-fluoro-4-iodo-phenylamino)-phenyl]-3H-[1,3,4]oxadiazol-2-one (10.0 g, 23.1 mmol) was dissolved in tetrahydrofuran (200 mL). To this solution was added, triethylamine (7 mL), (trimethylsilyl)acetylene (7 mL, 50 mmol), dichlorobis(triphenylphosphine)-palladium(II) (0.84 g, 1.2 mmol), and copper(I) iodide (0.24 g, 1.3 mmol). The resultant reaction mixture was stirred 17 h at ambient temperature and was concentrated in vacuo. The residue was dissolved in diethyl ether (500 mL) and washed with 5% aqueous hydrochloric acid (250 mL) and water (200 mL with water wash pH˜5 after aqueous layer separated). The organic phase was dried over magnesium sulfate and concentrated in vacuo. Chromatography on silica gel (0%→50% acetone in hexanes) afforded 5-[3,4-difluoro-2-(2-fluoro-4-trimethylsilanylethynyl-phenylamino)-phenyl]-3H-[1,3,4]oxadiazol-2-one (9.4 g, 100% yield) as a dark foam.

Step 2

A solution of 5-[3,4-difluoro-2-(2-fluoro-4-trimethylsilanylethynyl-phenylamino)-phenyl]-3H-[1,3,4]oxadiazol-2-one (8.9 g, 21 mmol) in isopropanol (250 mL) was treated with ethanolamine (1.65 g, 27 mmol) and the stirring mixture was brought to reflux for 23 hours. The reaction mixture was concentrated in vacuo to a dark viscous residue (12.3 g). The crude product was purified by flash chromatography. Elution with methanol (0%-8%) in dichloromethane afforded 3,4-difluoro-2-(2-fluoro-4-trimethylsilanylethynyl-phenylamino)-benzoic acid N′-(4-hydroxyethylaminocarbonyl)-hydrazide (5.3 g, 54% yield) as a tan amorphous solid.

Step 3

A solution comprised of 3,4-difluoro-2-(2-fluoro-4-trimethylsilanylethynyl-phenylamino)-benzoic acid N′-(4-hydroxyethylaminocarbonyl)-hydrazide (4.7 g, 10 mmol), triethylamine (2.80 mL, 20 mmol), carbon tetrachloride (1.93 mL, 20 mmol), and polystyrene-bound triphenylphosphine resin (15.0 g) in dichloromethane (250 mL) was brought to reflux for 1.5 hours. The mixture was vacuum filtered and the polymer resin was washed with 25% methanol in dichloromethane (1 L). The filtrate and wash were combined and concentrated in vacuo to afford an orange-brown foam (6.4 g) that was purified by flash chromatography. Elution with methanol (0-5%) in dichloromethane afforded 2-{5-[3,4-difluoro-2-(2-fluoro-4-trimethylsilanylethynyl-phenylamino)-phenyl]-[1,3,4]oxadiazol-2-ylamino}-ethanol (3.1 g, 69% yield) as a pale yellow amorphous foam.

Step 4

A solution of 2-{5-[3,4-difluoro-2-(2-fluoro-4-trimethylsilanylethynyl-phenylamino)-phenyl]-[1,3,4]oxadiazol-2-ylamino}-ethanol (3.0 g, 6.7 mmol) in methanol (200 mL) was treated with cesium fluoride (7.3 g, 48 mmol). The reaction mixture was stirred under a nitrogen atmosphere at ambient temperature for 20 hours and was concentrated in vacuo. The concentrate was taken up into diethyl ether (300 mL) and the ether layer was washed with water (300 mL), was dried over magnesium sulfate, and was concentrated in vacuo to a white amorphous solid. The amorphous product was recrystallized from chloroform to afford 2-{5-[2-(4-ethynyl-2-fluoro-phenylamino)-3,4-difluoro-phenyl]-[1,3,4]oxadiazol-2-ylamino}-ethanol as white fluffy crystals (2.2 g, 87% yield); mp 115.9-116.5° C.; ¹H NMR (400 MHz, DMSO-d₆) δ 8.83 (s, 1 H), 7.94 (t, J=5.9 Hz, 1 H), 7.52 (m, 1 H), 7.34 (dd, J=12.0, 1.7 Hz, 1 H), 7.27 (m, 1 H), 7.14 (dd, J=8.4, 1.5 Hz, 1 H), 6.86 (m, 1 H), 4.73 (t, J=5.6 Hz, 1 H), 4.11 (s, 1 H), 3.48 (dt, J=5.9, 5.6 Hz, 2 H), 3.22 (dt, J=5.9, 5.9 Hz, 2 H); MS(APCI+) 375.1 (M+1, 100), MS(APCI−) 373.1 (M-1, 61). C26ELSA IC₅₀=0.003 μM

### EXAMPLE 141

### [2,3-Difluoro-6-(5-methyl-4H-[1,2,4]triazol-3-yl)-phenyl]-(4-ethyl-2-fluoro-phenyl)-amine

Step 1

2-(4-Ethyl-2-fluoro-phenylamino)-3,4-difluoro-benzoic Acid Hydrazide

To a stirring solution of 2-(4-Ethyl-2-fluoro-phenylamino)-3,4-difluoro-benzoic acid (3.46 g, 11.7 mmol) in THF (40 mL) was added 4-methyl morpholine (1.67 mL, 15.2 mmol). This was cooled to 10° C. and isobutyl chloroformate 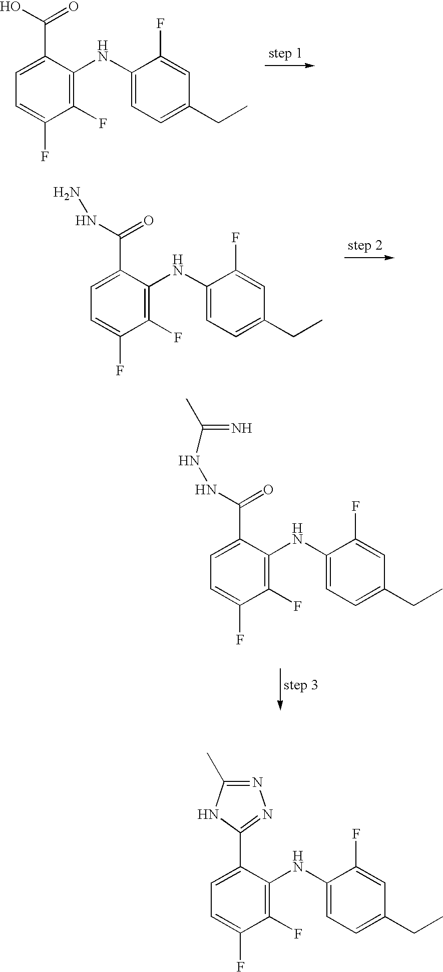
(2.17 mL, 16.7 mmol) added. A white precipitate in a yellow solution was formed. This was stirred at 10° C. for 10 minutes and filtered under reduced pressure. The filtrate was added to a solution of hydrazine (2.01 mL, 58.6 mmol) in THF (40 ml) at 0° C. and stirred for 45 minutes. The reaction mixture was poured into ethyl acetate (250 mL) washed successively with ammonium chloride (100 mL) and brine (100 mL) . The organic layer was separated dried (MgSO₄) and the solvent removed in vacuo to give 2-(4-Ethyl-2-fluoro-phenylamino)-3,4-difluoro-benzoic acid hydrazide as a yellow oil this solidified on drying to give a yellow solid.

Yield=3.33 g, 92%

MS(APCI+)=310 (MH+)

m.p.=97.9-98.5° C.

Step 2

2-(4-Ethyl-2-fluoro-phenylamino)-3,4-difluoro-benzoic Acid N′-(1-imino-ethyl)-hydrazide

To a stirred solution of 2-(4-Ethyl-2-fluoro-phenylamino)-3,4-difluoro-benzoic acid hydrazide (1 g, 3.24 mmol) in THF (20 mL) at 0° C. was added ethyl acetimidate hydrochloride salt (480 mg, 3.88 mmol) and triethyl amine (0.587 mL, 4.21 mmol). This was stirred at 0° C. for 1.5 h. followed by stirring at room temperature for 48 h. The reaction mixture was poured into water, neutralized with dilute HCL aq. and extracted with ethyl acetate (250 mL). The organic layer was dried (MgSO₄) and the solvent removed in vacuo. The residue was purified by chromatography using 5% methanol in dichloromethane to give 2-(4-Ethyl-2-fluoro-phenylamino)-3,4-difluoro-benzoic acid N′-(1-imino-ethyl)-hydrazide as a pale pink solid. Yield=715 mg, 63%

MS(APCI+)=351 (MH+)

Step 3

[2,3-Difluoro-6-(5-methyl-4H-[1,2,4]triazol-3-yl)-phenyl]-(4-ethyl-2-fluoro-phenyl)-amine

A solution of 2-(4-Ethyl-2-fluoro-phenylamino)-3,4-difluoro-benzoic acid N′-(1-imino-ethyl)-hydrazide in xylene (15 mL) was heated in a sealed tube at 120° C. for 12 hours. The solvent was removed in vacuo and the residue was purified by chromatography using 5% methanol in dichloromethane to give [2,3-Difluoro-6-(5-methyl-4H-[1,2,4]triazol-3-yl)-phenyl]-(4-ethyl-2-fluoro-phenyl)-amine as a white solid.

Yield=136 mg, 20%

MS(APCI+)=333 (MH+)

¹NMR (400 MHz; DMSO-d₆) 8.74(1H, s), 7.66(1H, m), 7.18-7.12(1H, m), 7.05(1H, dd, J=12.4 Hz, 1.7 Hz), 6.98-6.87(2H, m) 2.51(5H, m) 1.11 (3H, t J=5.76)

### EXAMPLE 142

### (4-Ethyl-2-fluoro-phenyl)-[6-(5-ethyl-[1,3,4]oxadiazol-2-yl)-2,3-difluoro-phenyl]-amine

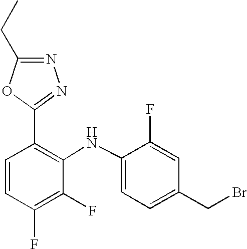

To a stirred solution of 2-(4-Ethyl-2-fluoro-phenylamino)-3,4-difluoro-benzoic acid hydrazide (800 mg, 2.59 mmol) in dioxane (5 mL) was added triethylorthopropionate (1.56 mL, 7.77 mmol) and methanesulfonic acid (0.038 mL, 0.52 mmol). This was heated at 110° C. for 20 minutes. The reaction mixture was poured into water and extracted with ethyl acetate (100 mL). The organic layer was dried (MgSO₄) and the solvent removed in vacuo. The residue was purified by chromatography using 5% methanol in dichloromethane to give (4-Ethyl-2-fluoro-phenyl)-[6-(5-ethyl-[1,3,4]oxadiazol-2-yl)-2,3-difluoro-phenyl]-amine as a white solid.

Yield=378 mg, 42%

MS(APCI+)=348 (MH+)

m.p.=82-83° C.

Anal. calcd/found for C₁₈H₁₆F₃N₃O: C 62.24/62.23, H 4.64/4.56, N 12.10/11.91

¹NMR (400 MHz; DMSO-d₆) 8.73(1H, s), 7.68(1H, m), 7.18-7.12(1H, m), 7.05(1H, dd, J=12.2 Hz, 1.7 Hz), 6.95-6.87(2H, m) 2.85(2H, q J=7.6 Hz), 2.51(2H, q J=7.6 Hz), 1.11 (6H, m)

### EXAMPLE 143

### [6-(5-Ethyl-[1,3,4]oxadiazol-2-yl)-2,3-difluoro-phenyl]-(2-fluoro-4-iodo-phenyl)-amine

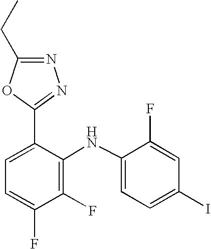

To a stirred solution of 3,4-Difluoro-2-(2-fluoro-4-iodo-phenylamino)-benzoic acid hydrazide (800 mg, 1.96 mmol) in dioxane (5 mL) was added triethylorthopropionate (1.19 mL, 5.89 mmol) and methanesulfonic acid (0.029 mL, 0.39 mmol). This was heated at 110° C. for 20 minutes. The reaction mixture was poured into water and extracted with ethyl acetate. The organic layer was dried (MgSO₄) and the solvent removed in vacuo. The residue was purified by chromatography using 5% methanol in dichloromethane to give [6-(5-Ethyl-[1,3,4]oxadiazol-2-yl)-2,3-difluoro-phenyl]-(2-fluoro-4-iodo-phenyl)-amine as a white solid. Yield=576 mg, 66%. MS(APCI+)=446 (MH+). m.p.=97-98° C. Anal. calcd/found for C₁₆H₁₁F₃IN₃O: C 43.17/43.36, H 2.49/2.22, N 9.44/9.28 C26ELSA % Inhibition@5 μM=88%

### EXAMPLE 144

### [6-(5-Ethyl-[1,3,4]oxadiazol-2-yl)-2,3-difluoro-phenyl]-(4-ethynyl-2-fluoro-phenyl)-amine

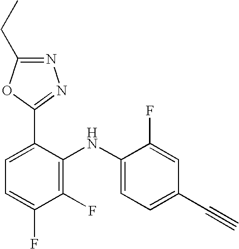

To a stirred solution of 2-(4-Ethynyl-2-fluoro-phenylamino)-3,4-difluoro-benzoic acid hydrazide (800 mg, 2.62 mmol) in dioxane (5 mL) was added triethylorthopropionate (1.65 mL, 8.24 mmol) and methanesulfonic acid (0.040 mL, 0.55 mmol). This was heated at 110° C. for 20 minutes. The reaction mixture was poured into water and extracted with ethyl acetate. The organic layer was dried (MgSO₄) and the solvent removed in vacuo. The residue was purified by chromatography using 5% methanol in dichloromethane to give [6-(5-Ethyl-[1,3,4]oxadiazol-2-yl)-2,3-difluoro-phenyl]-(4-ethynyl-2-fluoro-phenyl)-amine as a white solid. Yield=370 mg, 41%. MS(APCI+)=344 (MH+) Anal. calcd/found for C₁₈H₁₂F₃N₃O: C 62.98/62.69, H 3.52/3.53, N 12.24/12.06 C26ELSA % Inhibition@5 μM=43%

### EXAMPLE 145

### {6-[5-(3-Bromo-propyl)-[1,3,4]oxadiazol-2-yl]-2,3-difluoro-phenyl}-(4-ethyl-2-fluoro-phenyl)-amine

C26ELSA % Inhibition@5 μM=13%

### EXAMPLE 146

### {2,3-Difluoro-6-[5-(3-morpholin-4-yl-propyl)-[1, 3,4]oxadiazol-2-yl]-phenyl}-(4-ethyl-2-fluoro-phenyl)-amine

To a stirred solution of 2-(4-Ethyl-2-fluoro-phenylamino)-3,4-difluoro-benzoic acid hydrazide (400 mg, 1.29 mmol) in dioxane (5 mL) was added trimethyl 4-bromo orthobutyrate (0.671 mL, 3.87 mmol) and methanesulfonic acid (0.019 mL, 0.26 mmol). This was heated at 110° C. for 20 minutes. Analysis by Mass spectrum revealed formation of {6-[5-(3-Bromo-propyl)-[1,3,4]oxadiazol-2-yl]-2,3-difluoro-phenyl}-(4-ethyl-2-fluoro-phenyl)-amine. Morpholine (0.338 mL, 3.87 mmol) was added and the reaction heated at reflux for an additional hour. The reaction mixture was poured into water and extracted with ethyl acetate. The organic layer was dried (MgSO₄) and the solvent removed in vacuo. The residue was purified by chromatography using 5%-10% methanol in dichloromethane to give fraction 1 {6-[5-(3-Bromo-propyl)-[1,3,4]oxadiazol-2-yl]-2,3-difluoro-phenyl}-(4-ethyl-2-fluoro-phenyl)-amine as a clear oil. Yield=215 mg, 38% MS(APCI+)=441 (MH+. ¹NMR (400 MHz; DMSO-d₆) 8.71(1H, s), 7.69(1H, m), 7.18-7.12(1H, m), 7.05(1H, dd, J=12.2 Hz, 1.7 Hz), 6.95-6.87(2H, m), 3.59 (2H, t, J=6.6 Hz), 3.00 (2H, t, J=7.3 Hz), 2.51(2H, q J=7.6 Hz), 2.18, (2H, quintet, J=6.8), 1.11(3H, t, J=7.6 Hz).

The third fraction was {2,3-Difluoro-6-[5-(3-morpholin-4-yl-propyl)-[1,3,4]oxadiazol-2-yl]-phenyl}-(4-ethyl-2-fluoro-phenyl)-amine

Yield=348 mg, 60%. MS(APCI+)=447 (MH+) C26ELSA % Inhibition@5 μM=6%

### EXAMPLE 147

### 3-(4-{2,3-Difluoro-6-[5-(2-morpholin-4-yl-ethylamino)-[1,3,4]oxadiazol-2-yl]-phenylamino}-3-fluoro-phenyl)-propan-1-ol

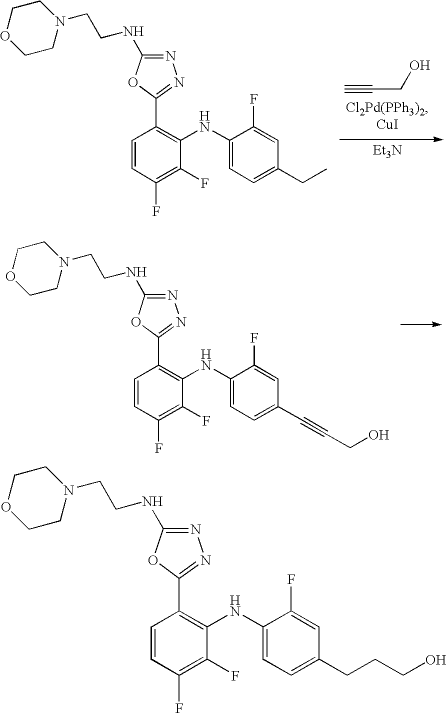

Step 1

To a stirring solution of {5-[3,4-Difluoro-2-(2-fluoro-4-iodo-phenylamino)-phenyl]-[1,3,4]oxadiazol-2-yl}-(2-morpholin-4-yl-ethyl)-amine (409 mg, 0.753 mmol), dichlorobis(triphenylphosphine)palladium(II) (14 mg, 0.02 mmol), and copper iodide (5.7 mg, 0.03 mmol) in triethylamine (8 mL) was added propargyl alcohol (0.052 mL, 0.89 mmol). The resultant mixture was stirred 18 h at ambient temperature and 5 h at 50-60° C. The solvent was removed in vacuo and the residue was diluted with ethyl acetate (50 mL) and washed with water (2×20 mL) and saturated brine (2×20 mL). The organics were dried over magnesium sulfate, concentrated under reduced pressure, and purified by silica gel chromatography to afford 3-(4-{2,3-Difluoro-6-[5-(2-morpholin-4-yl-ethylamino)-[1,3,4]oxadiazol-2-yl]-phenylamino}-3-fluoro-phenyl)-prop-2-yl-1-ol (198 mg, 56% yield).

Step 2

A solution of 3-(4-{2,3-Difluoro-6-[5-(2-morpholin-4-yl-ethylamino)-[1,3,4]oxadiazol-2-yl]-phenylamino}-3-fluoro-phenyl)-prop-2-yl-1-ol (198 mg, 0.42 mmol) in anhydrous tetrahydrofuran (16 mL) was hydrogenated (6900 psi) over 10% palladium/carbon (50 mg) for 8 h. The reaction mixture was filtered and concentrated to an oil that solidified upon standing. Recrystallization from ether-dichloromethane afforded 3-(4-{2,3-Difluoro-6-[5-(2-morpholin-4-yl-ethylamino)-[1,3,4]oxadiazol-2-yl]-phenylamino}-3-fluoro-phenyl)-propan-1-ol (130 mg, 65% yield). An analytical sample was prepared by recrystallization from dichloromethane-heptane: m. p. 102-106° C.; ¹H NMR (400 MHz, CDCl₃) δ 8.95 (s, 1 H), 7.44 (ddd, J=9.0, 5.4, 2.2 Hz, 1 H), 6.96-6.84 (m, 3 H), 6.74 (dt, J=6.8, 9.2 Hz, 1 H), 5.45 (t, J=5.0 Hz, 1 H), 3.72 (t, J=4.6 Hz, 4 H), 3.66 (t, J=6.2 Hz, 2 H), 3.48 (dt, J=6.1, 5.4 Hz, 2 H), 2.70-2.60 (m, 4 H), 2.49 (apparent t, J=4.4 Hz, 4 H), 1.86 m (2 H). Anal. Cald/Found for C₂₃H₂₆F₃N₅O₃: C, 57.86/58.00; H, 5.49/5.35; N, 14.67/14.58.

C26ELSA IC₅₀=34.0 μM

### EXAMPLE 148

### {5-[2-(4-Ethynyl-2-fluoro-phenylamino)-3,4-difluoro-phenyl]-[1,3,4]oxadiazol-2-yl}-(2-morpholin-4-yl-ethyl)-amine

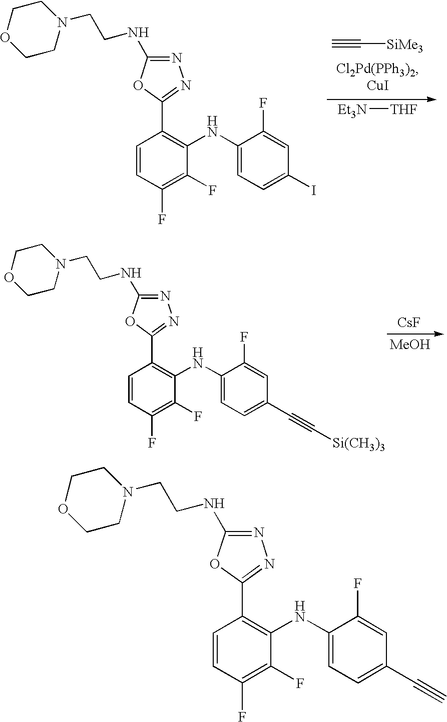

Step 1

To a stirring solution of {5-[3,4-Difluoro-2-(2-fluoro-4-iodo-phenylamino)-phenyl]-[1,3,4]oxadiazol-2-yl}-(2-morpholin-4-yl-ethyl)-amine (296 mg, 0.543 mmol) in tetrahydrofuran-triethylamine (1:3, 8 mL) was added (trimethylsilyl)acetylene (0.150 mL, 1.06 mmol), dichlorobis(triphenylphosphine)palladium(II) (20 mg, 0.028 mmol), and copper iodide (10 mg, 0.053 mmol). The resultant mixture was stirred 5 h at ambient temperature, diluted with ethyl acetate (50 mL) and washed with water (2×20 mL) and saturated brine (2×20 mL). The organics were dried over magnesium sulfate, concentrated under reduced pressure, and purified by silica gel chromatography. Elution with dichloromethane-methanol (9:1) afforded {5-[3,4-Difluoro-2-(2-fluoro-4-trimethylsilanylethynyl-phenylamino)-phenyl]-[1,3,4]oxadiazol-2-yl)-(2-morpholin-4-yl-ethyl)-amine (230 mg, 82% yield) as a brown-colored solid.

Step 2

A solution of {5-[3,4-Difluoro-2-(2-fluoro-4-trimethylsilanylethynyl-phenylamino)-phenyl]-[1,3,4]oxadiazol-2-yl}-(2-morpholin-4-yl-ethyl)-amine (129 mg, 0.25 mmol) in anhydrous methanol (4 mL) was treated with cesium fluoride (115 mg, 0.75 mmol). The resultant solution was stirred 48 h at ambient temperature. The reaction mixture was diluted with 60 mL of ethyl acetate and was washed with water (2×10 mL) and saturated brine (20 mL). The organics were dried over magnesium sulfate, concentrated in vacuo and chromatographed on silica gel. Gradient elution with 3-8% methanol in dichloromethane afforded {5-[2-(4-Ethynyl-2-fluoro-phenylamino)-3,4-difluoro-phenyl]-[1,3,4]oxadiazol-2-yl)}-(2-morpholin-4-yl-ethyl)-amine (92 mg, 83% yield) as a foam that solidified on standing. Recrystallization from ethyl acetate-hexanes afforded a white solid: m.p. 146-148° C.; ¹H NMR (400 MHz, CDCl₃) □ 9.03 (s, 1 H), 7.46 (m, 1 H), 7.21-7.13 (m, 2 H), 6.88-6.79 (m, 2 H), 5.53 (br s, 1 H), 3.70 (apparent t, J=4.3 Hz, 4 H), 3.48 (dt, J=5.9, 5.4 Hz, 2 H), 3.02 (s, 1 H), 2.64-2.60 (m, 4 H), 2.48 (m, 4 H). Anal. Cald/Found for C₂₂H₂₀F₃N₅O₂: C, 59.59/59.54; H, 4.55/4.50; N, 15.79/15.61.

C26ELSA IC₅₀=0.063 μM

### EXAMPLE 149

### 1-(4-{2,3-Difluoro-6-[5-(2-morpholin-4-yl-ethylamino)-[1,3,4]oxadiazol-2-yl]-phenylamino}-3-fluoro-phenyl)-ethanone

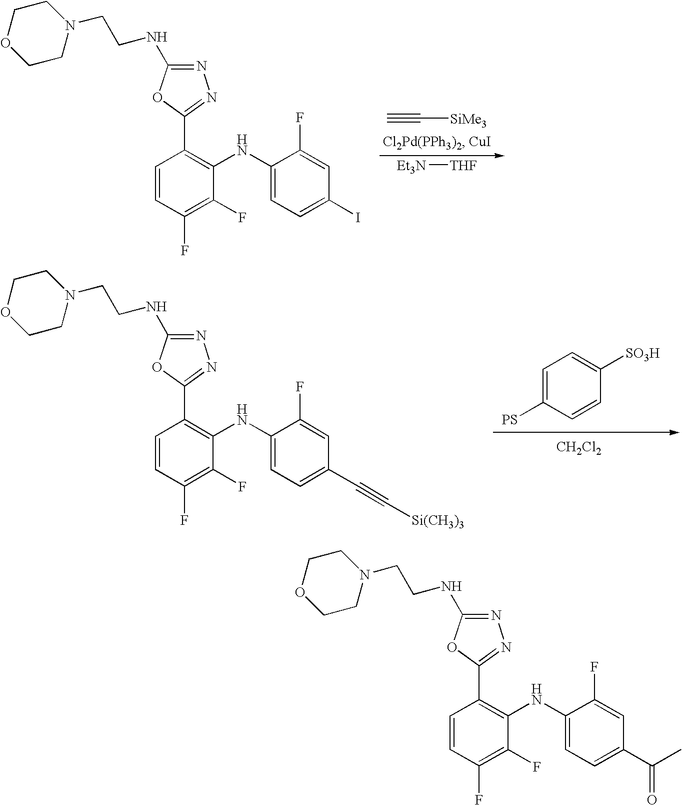

Step 1

To a stirring solution of {5-[3,4-Difluoro-2-(2-fluoro-4-iodo-phenylamino)-phenyl]-[1,3,4]oxadiazol-2-yl)-(2-morpholin-4-yl-ethyl)-amine (296 mg, 0.543 mmol) in tetrahydrofuran-triethylamine (1:3, 8 mL) was added (trimethylsilyl)acetylene (0.150 mL, 1.06 mmol), dichlorobis(triphenylphosphine) palladium(II) (20 mg, 0.028 mmol), and copper iodide (10 mg, 0.053 mmol). The resultant mixture was stirred 5 h at ambient temperature, diluted with ethyl acetate (50 mL) and washed with water (2×20 mL) and saturated brine (2×20 mL). The organics were dried over magnesium sulfate, concentrated under reduced pressure, and purified by silica gel chromatography. Elution with dichloromethane-methanol (9:1) afforded {5-[3,4-Difluoro-2-(2-fluoro-4-trimethylsilanylethynyl-phenylamino)-phenyl]-[1,3,4]oxadiazol-2-yl}-(2-morpholin-4-yl-ethyl)-amine (230 mg, 82% yield) as a brown-colored solid.

Step 2

A solution of {5-[3,4-Difluoro-2-(2-fluoro-4-trimethylsilanylethynyl-phenylamino)-phenyl]-[1,3,4]oxadiazol-2-yl}-(2-morpholin-4-yl-ethyl)-amine (72 mg, 0.14 mmol) in dichloromethane (3 mL) was treated with polymer-supported toluenesulfonic acid (0.58 g, 1.5 mmol/g). The resultant mixture was stirred at ambient temperature for 24 h, then heated at reflux for 7 h. The cooled reaction mixture was filtered and the product was removed form the resin with a solution comprised of 30% aqueous ammonium hydroxide/methanol/dichloromethane (1:10:100, 50 mL). The filtrate was concentrated under reduced pressure and purified by silica gel chromatography. Elution with 10% methanol in dichloromethane afforded 1-(4-{2,3-Difluoro-6-[5-(2-morpholin-4-yl-ethylamino)-[1,3,4]oxadiazol-2-yl]-phenylamino}-3-fluoro-phenyl)-ethanone (53 mg, 82% yield) as a white foam: ¹H NMR (400 MHz, CDCl₃) δ 9.15 (s, 1 H), 7.72-6.63 (m, 2 H), 7.52 (m, 1 H), 6.95 (dt, J=7.0, 9.0 Hz, 1 H), 6.87 (dt, J=7.1, 8.0 Hz, 1 H), 5.49 (br s, 1 H), 3.72 (br s, 4 H), 3.49 (apparent q, J=5.6 Hz, 2 H), 2.64 (t, J=5.4 Hz, 2 H), 2.54 (s, 3 H), 2.50 (br s, 4 H); MS(APCI+)=462.1, MS(APCI−)=460.1. C26ELSA % Inhib@5 μM=14%

C26ELSA % Inhib@0.5 μM=2%

### EXAMPLE 150

### 3-{5-[3,4-Difluoro-2-(2-fluoro-4-iodo-phenylamino)-phenyl]-[1,3,4]oxadiazol-2-ylamino}-propane-1,2 (R)-diol

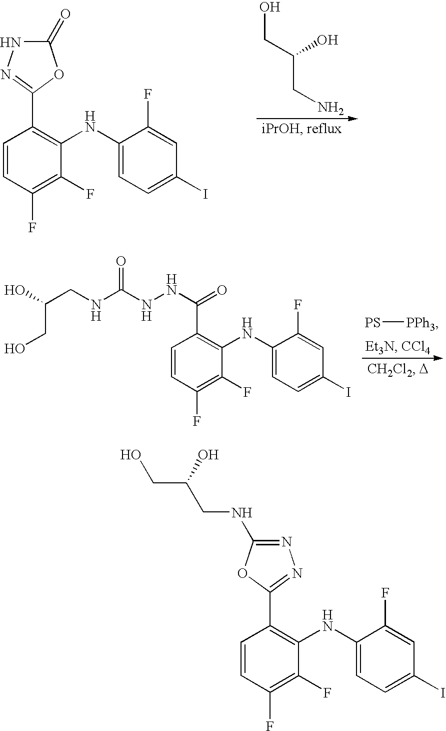 Step 1

5-[3,4-Difluoro-2-(2-fluoro-4-iodo-phenylamino)-phenyl]-3H-[1,3,4]oxadiazol-2-one (16.0 g, 37 mmol) and (R)-(+)-3-amino-1,2-propanediol (4.25 g, 46.7 mmol) were combined in isopropanol (150 mL) and heated to reflux under a nitrogen atmosphere. After 18 h, the reaction mixture was cooled to ambient temperature and concentrated to about ¼ volume. The crude reaction mixture was diluted with an approximately equal volume of ether, and crystallization was induced by scratching with a glass rod. The crystals were filtered, washed with ether, and dried in vacuo to afford the urea product as a crystalline solid (16.37 g, 84% yield).

Step 2

The urea from Step 1 (16.37 g, 31.2 mmol) was combined with PS-triphenylphosphine (Argonaut Technologies, 53.6 mmol P) and triethylamine (7.6 mL, 54 mmol) in dichloromethane (300 mL). Carbon tetrachloride (5.2 mL, 54 mmol) was added and the reaction mixture was heated at reflux for 2.5 h under a nitrogen atmosphere. The reaction mixture was filtered, and the resin was washed with dichloromethane (500 mL) and ethyl acetate (400 mL). The combined filtrate was washed with water (3×100 mL) and saturated brine (100 mL), was dried over magnesium sulfate and was concentrated in vacuo to an oily foam (fraction A). The resin was further washed with dichloromethane-methanol (3:1, 1 L) and this filtrate was combined with fraction A and concentrated in vacuo. The product began to crystallize upon concentration and was isolated by filtration. Two additional crops of product were obtained by further concentration of the mother liquors and collection of the solids to afford a white powder (10.56 g). Concentration of the mother liquors afforded an additional 5.97 g of product. Recrystallization of the solid (10.56 g) from methanol afforded analytically pure 3-{5-[3,4-Difluoro-2-(2-fluoro-4-iodo-phenylamino)-phenyl]-[1,3,4]oxadiazol-2-ylamino}-propane-1,2 (R)-diol (8.05 g): m.p. 174-176° C.; ¹H NMR (400 MHz, DMSO-d₆) δ 8.84 (s, 1 H), 7.94 (t, J=5.9 Hz, 1 H), 7.65 (dd, J=10.7, 1.5 Hz, 1 H), 7.56 (m, 1 H), 7.42 (d, J=8.3 Hz, 1 H), 7.27 (apparent q, J=9.0 Hz, 1 H), 6.80 (m, 1 H), 4.86 (d, J=5.1 Hz, 1 H), 4.62 (t, J=5.6 Hz, 1 H), 3.66 (m, 1 H), 3.36 (m, 3 H), 3.14 (m, 1 H). Anal. Cald/Found for C₁₇H₁₄F₃IN₄O₃: C, 40.34/40.39; H, 2.79/2.44; N, 11.07/10.93; F, 11.07/11.47; I, 25.07/24.78. [α]=+8.9° (DMSO, c=6.3).

C26ELSA IC₅₀=0.092 μM

### EXAMPLE 151

### 3-{5-[2-(4-Ethynyl-2-fluoro-phenylamino)-3,4-difluoro-phenyl]-[1,3,4]oxadiazol-2-ylamino}-propane-1,2(R)-diol

Step 1

3-{5-[3,4-Difluoro-2-(2-fluoro-4-iodo-phenylamino)-phenyl]-[1,3,4]oxadiazol-2-ylamino}-propane-1,2 (R)-diol (1.92 g, 3.79 mmol) was dissolved in tetrahydrofuran (25 mL). To this solution was added, triethylamine (25 mL), (trimethylsilyl)acetylene (1.07 mL, 7.57 mmol), dichlorobis(triphenylphosphine)-palladium(II) (137 mg, 0.195 mmol), and copper(I) iodide (102 mg, 0.53 mmol). The resultant reaction mixture was stirred 6 h at ambient temperature, filtered through a pad of Celite. The Filtered salts were thoroughly washed with tetrahydrofuran and the filtrate was concentrated in vacuo. The residue was diluted with ethyl acetate (200 mL) and washed with water (2×50 mL) and saturated brine (50 mL), dried over magnesium sulfate and concentrated in vacuo. Chromatography on silica gel (10%→30% methanol in dichloromethane) afforded 3-{5-[3,4-Difluoro-2-(2-fluoro-4-trimethylsilanylethynyl-phenylamino)-phenyl]-[1,3,4]oxadiazol-2-ylamino}-propane-1,2(R)-diol (1.44 g, 80% yield) as a brown solid.

Step 2

A solution of 3-{5-[3,4-difluoro-2-(2-fluoro-4-trimethylsilanylethynyl-phenylamino)-phenyl]-[1,3,4]oxadiazol-2-ylamino}-propane-1,2(R)-diol (1.44 g, 3.02 mmol) in methanol (30 mL) was treated with cesium fluoride (1.35 g, 8.89 mmol) and glacial acetic acid (0.19 mL, 3.17 mmol). The resultant solution was stirred at ambient temperature for 18 h. Water (60 mL) was added and the resultant suspension was stirred vigorously for 5 h. The solid was collected by filtration and washed with water-methanol (2:1, 90 mL). The brown solid was dissolved in acetone and chromatographed on silica gel. Elution with methanol-dichloromethane (10%-30%) afforded 3-{5-[2-(4-Ethynyl-2-fluoro-phenylamino)-3,4-difluoro-phenyl]-[1,3,4]oxadiazol-2-ylamino}-propane-1,2(R)-diol (775 mg, 63% yield) as a cream-colored solid: ¹H NMR (400 MHz, DMSO-d₆) δ 8.91 (s, 1 H), 7.94 (t, J=5.9 Hz, 1 H), 7.58 (ddd, J=9.0, 5.7, 1.9 Hz, 1 H), 7.40 (dd, J=12.0, 1.7 Hz, 1 H), 7.33 (dt, J=7.3, 9.3 Hz, 1 H), 7.21 (dd, J=8.5, 1.7 Hz, 1 H), 6.93 (td, J=8.8, 5.4 Hz, 1 H), 4.86 (d, J=5.1 Hz, 1 H), 4.63 (t, J=5.7 Hz, 1 H), 4.17 (s, 1 H), 3.64 (m, 1 H), 3.36 (m, 3 H), 3.15 (m, 1 H);

MS(APCI+)=405.1, MS(APCI−)=403.1.

C26ELSA IC₅₀=0.12 μM

### EXAMPLE 152

### 3-{5-[2-(4-Ethyl-2-fluoro-phenylamino)-3,4-difluoro-phenyl]-[1,3,4]oxadiazol-2-ylamino}-propane-1,2(R)-diol

A solution of 3-{5-[2-(4-Ethynyl-2-fluoro-phenylamino)-3,4-difluoro-phenyl]-[1,3,4]oxadiazol-2-ylamino}-propane-1,2(R)-diol (0.38 g, 0.94 mmol) in ethanol-tetrahydrofuran (1:1, 50 mL) was hydrogenated (4295 psi) over Raney Nickel (0.65 g) for 40 h. The reaction mixture was filtered and the filtrate was concentrated to a brown oil that was chromatographed on silica gel. Elution with methanol-dichloromethane (5→25%) afforded 3-{5-[2-(4-Ethyl-2-fluoro-phenylamino)-3,4-difluoro-phenyl]-[1,3,4]oxadiazol-2-ylamino}-propane-1,2(R)-diol (251 mg, 65% yield) as a white solid. Recrystallization from acetone-hexanes afforded analytically pure material: m.p. 150-152° C. (dec); ¹H NMR (400 MHz, DMSO-d₆) δ 8.88 (s, 1 H), 7.94 (t, J=5.3 Hz, 1 H), 7.53 (ddd, J=9.0, 5.7, 1.9 Hz, 1 H), 7.17 (dt, J=7.1, 9.4 Hz, 1 H), 7.12 (br d, J=11.5 Hz, 1 H), 7.02-6.93 (m, 2 H), 4.87 (d, J=4.9 Hz, 1 H), 4.63(t, J=5.6 Hz, 1 H), 3.67 (m, 1 H), 3.37 (m, 3 H), 3.17 (m, 1 H), 2.56 (q, J=7.6 Hz, 2 H), 1.17 (t, J=7.6 Hz, 3 H). Anal. Cald/Found for C₁₉H₁₉F₃N₄O₃: C, 55.88/55.84; H, 4.69/4.32; N, 13.72/13.64.

C26ELSA IC₅₀=2.4 μM

### EXAMPLE 153

### 3-{5-[3,4-Difluoro-2-(2-fluoro-4-iodo-phenylamino)-phenyl]-[1,3,4]oxadiazol-2-ylamino}-propane-1,2 (S)-diol

3-{5-[3,4-Difluoro-2-(2-fluoro-4-iodo-phenylamino)-phenyl]-[1,3,4]oxadiazol-2-ylamino}-propane-1,2 (S)-diol (9.99 g) was prepared from 5-[3,4-Difluoro-2-(2-fluoro-4-iodo-phenylamino)-phenyl]-3H-[1,3,4]oxadiazol-2-one (15.4 g, 35.5 mmol) and (S)-(−)-3-amino-1,2-propanediol (4.05 g, 44.5 mmol) by the procedure described for 3-{5-[3,4-Difluoro-2-(2-fluoro-4-iodo-phenylamino)-phenyl]-[1,3,4]oxadiazol-2-ylamino}-propane-1,2 (R)-diol. A white solid: ¹H NMR (400 MHz, DMSO-d₆) δ 8.84 (s, 1 H), 7.94 (t, J=5.9 Hz, 1 H), 7.65 (dd, J=10.7, 1.5 Hz, 1 H), 7.56 (m, 1 H), 7.42 (d, J=8.3 Hz, 1 H), 7.27 (apparent q, J=9.0 Hz, 1 H), 6.80 (m, 1 H), 4.86 (d, J=5.1 Hz, 1 H), 4.62 (t, J 5.6 Hz, 1 H), 3.66 (m, 1 H), 3.36 (m, 3 H), 3.14 (m, 1 H).

### EXAMPLE 154

### 2-{5-[3,4-Difluoro-2-(2-fluoro-4-iodo-phenylamino)-phenyl]-[1,3,4]oxadiazol-2-ylamino}-propane-1,3-diol

Step 1

5-[3,4-Difluoro-2-(2-fluoro-4-iodo-phenylamino)-phenyl]-3H-[1,3,4]oxadiazol-2-one (15.2 g, 35.1 mmol) and 2-amino-1,3-propanediol (4.13 g, 45.3 mmol) were combined in isopropanol (150 mL) and heated to reflux under a nitrogen atmosphere. After 5 days, the reaction mixture was cooled to ambient temperature and concentrated to about ⅓ volume. Crystallization was induced by scratching with a glass rod. The crystals were filtered, washed with isopropanol, and dried in vacuo to afford the urea product as a crystalline solid (16.00 g, 87% yield).

Step 2

The urea from Step 1 (16.00 g, 30.5 mmol) was combined with PS-triphenylphosphine (Argonaut Technologies, 46.5 mmol P) and triethylamine (7.3 mL, 52 mmol) in dichloromethane (300 mL). Carbon tetrachloride (5.0 mL, 52 mmol) was added and the reaction mixture was heated at reflux for 8 h under a nitrogen atmosphere. Additional portions of PS-triphenylphoshine (8.97 g, 15.2 mmol P), carbon tetrachloride (2.0 mL, 21 mmol), and triethylamine (2.5 mL, 17.8 mmol) were added and the reaction mixture was heated at reflux an additional 5 h. The reaction mixture was filtered, and the resin was washed with dichloromethane (500 mL). The combined filtrate was washed with water (2×100 mL) and saturated brine (100 mL), was dried over magnesium sulfate and was concentrated in vacuo to a foam (fraction A). The resin was further washed with tetrahydrofuran (1 L) and tetrahydrofuran-methanol (3:1, 1 L) and this filtrate was concentrated in vacuo and combined with fraction A. The product was obtained by chromatography on silica gel. Elution with 5-25% methanol in dichloromethane afforded pure 2-{5-[3,4-Difluoro-2-(2-fluoro-4-iodo-phenylamino)-phenyl]-[1,3,4]oxadiazol-2-ylamino}-propane-1,3-diol (10.75 g, 70% yield) as an off-white foam. Crystallization from dichloromethane and drying in vacuo@40° C. afforded an analytically pure white solid: m.p. 110-114° C. (dec); ¹H NMR (400 MHz, DMSO-d₆) δ 8.84 (s, 1 H), 7.79 (d, J=7.6 Hz, 1 H), 7.64 (dd, J=10.7, 1.9 Hz, 1 H), 7.54 (ddd, J=9.0, 5.6, 1.8 Hz, 1 H), 7.41 (br d, J=8.4 Hz, 1 H), 7.30 (td, J=9.5, 6.9 Hz, 1 H), 6.80 (dt, J=5.1, 8.8 Hz, 1 H), 4.73 (t, J=5.4 Hz, 2 H), 3.52 (m, 5 H). Anal. Cald/Found for C₁₇H₁₄F₃IN₄O₃: C, 40.34/40.17; H, 2.79/2.44; N, 11.07/10.98.

C26ELSA IC₅₀=0.11 μM

### EXAMPLE 155

### 2-{5-[2-(4-Ethynyl-2-fluoro-phenylamino)-3,4-difluoro-phenyl]-[1,3,4]oxadiazol-2-ylamino}-propane-1,3-diol

Step 1

A solution of 2-{5-[3,4-Difluoro-2-(2-fluoro-4-iodo-phenylamino)-phenyl]-[1,3,4]oxadiazol-2-ylamino}-propane-1,3-diol (3.5 g, 6.91 mmol) in tetrahydrofuran-triethylamine (1:1, 40 mL) was added (trimethylsilyl)acetylene (1.95 mL, 13.8 mmol), dichlorobis(triphenylphosphine)-palladium(II) (239 mg, 0.340 mmol), and copper(I) iodide (163 mg, 0.856 mmol). The resultant reaction mixture was stirred 4 h at ambient temperature, filtered through a pad of Celite. The filtered salts were washed with ethyl acetate (300 mL) and the combined filtrate was washed with water (2×100 mL) and saturated brine (100 mL), dried over magnesium sulfate and concentrated in vacuo. Chromatography on silica gel (5%→25% methanol in dichloromethane) afforded 2-{5-[3,4-Difluoro-2-(2-fluoro-4-trimethylsilanylethynyl-phenylamino)-phenyl]-[1,3,4]oxadiazol-2-ylamino}-propane-1,3-diol (2.74 g, 83% yield) as a brown foam.

Step 2

A solution of 2-{5-[3,4-Difluoro-2-(2-fluoro-4-trimethylsilanylethynyl-phenylamino)-phenyl]-[1,3,4]oxadiazol-2-ylamino}-propane-1,3-diol (2.74 g, 5.75 mmol) in methanol (50 mL) was treated with cesium fluoride (2.69 g, 17.7 mmol) and glacial acetic acid (0.35 mL, 6.13 mmol). The resultant solution was stirred at ambient temperature for 15 h. The reaction mixture was concentrated in vacuo and partitioned between ethyl acetate (200 mL) and water (100 mL). The organics were washed with saturated aqueous sodium bicarbonate (50 mL) and saturated brine (50 mL), dried over magnesium sulfate, concentrated under reduced pressure and chromatographed on silica gel. Elution with 5→25% methanol in dichloromethane afforded a brown oil upon concentration. Addition of dichloromethane (30 mL) and scratching with a glass rod induced crystallization. The straw-colored crystals were collected and dried in vacuo@40° C. to afford 2-{5-[2-(4-ethynyl-2-fluoro-phenylamino)-3,4-difluoro-phenyl]-[1,3,4]oxadiazol-2-ylamino}-propane-1,3-diol: ¹H NMR (400 MHz, DMSO-d₆) δ 8.92 (s, 1 H), 7.81 (d, J=7.3 Hz, 1 H), 7.57 (ddd, J=9.0, 5.7, 1.8 Hz, 1 H), 7.40 (dd, J=12.0, 1.7 Hz, 1 H), 7.33 (td, J=9.4, 7.3 Hz, 1 H), 7.20 (dd, J=8.5, 1.6 Hz, 1 H), 6.93 (td, J=8.8, 5.4 Hz, 1 H), 4.74 (t, J=5.4 Hz, 2 H), 4.17 (s, 1 H), 3.53 (m, 5 H). Anal. Cald/Found for C₁₉H₁₅F₃N₄O₃—(CH₂Cl₂)_{0.2}: C, 54.73/54.34; H, 3.68/3.30; N, 13.30/12.99. MS(APCI+)=405.1, MS(APCI−)=403.1.

### EXAMPLE 156

### {5-[3,4-Difluoro-2-(2-fluoro-4-vinyl-phenylamino)-phenyl]-[1,3,4]oxadiazol-2-yl}-(2-morpholin-4-yl-ethyl)-amine

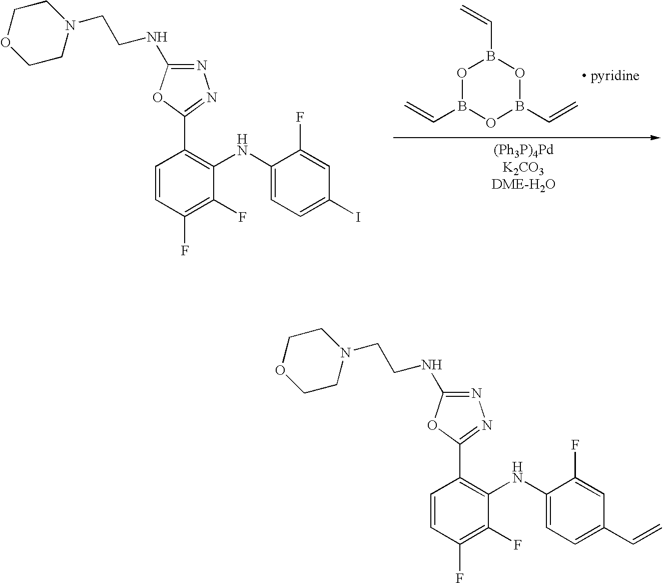

To a stirring solution of {5-[3,4-Difluoro-2-(2-fluoro-4-iodo-phenylamino)-phenyl]-[1,3,4]oxadiazol-2-yl}-(2-morpholin-4-yl-ethyl)-amine (2.80 g, 5.13 mmol) and 2,4,6-Trivinyl-cyclotriboroxane pyridine complex (1.12 g, 4.65 mmol) in dimethoxyethane (30 mL) and water (10 mL) was added potassium carbonate (699 mg, 5.06 mmol) and tetrakis(triphenylphosphine)palladium (260 mg, 0.23 mmol). The reaction mixture was heated at reflux for 2 h, diluted with ethyl acetate (100 mL) and washed with water (2×20 mL) and saturated brine (2×20 mL). The organics were dried over magnesium sulfate, concentrated under reduced pressure, and purified by silica gel chromatography. Gradient elution with dichloromethane-15% methanol/dichloromethane afforded the product as a pale yellow foam (1.55 g) contaminated with ca. 0.15 mol % triphenylphosphine oxide and 10 mol % 2,4,6-Trivinyl-cyclotriboroxane pyridine complex. An analytically pure sample of {5-[3,4-Difluoro-2-(2-fluoro-4-vinyl-phenylamino)-phenyl]-[1,3,4]oxadiazol-2-yl}-(2-morpholin-4-yl-ethyl)-amine was prepared by dissolution of the above mixture in tetrahydrofuran-water (1:1, 9 mL) and treatment with sodium perborate monohydrate (160 mg, 1.61 mmol) for 24 h. The reaction mixture was diluted with water and extracted with dichloromethane (3×25 mL) and ethyl acetate (3×25 mL). The combined extracts were dried over magnesium sulfate and concentrated in vacuo. Silica gel chromatography afforded a white solid (120 mg). Recrystallization from ethyl acetate afforded analytically pure {5-[3,4-Difluoro-2-(2-fluoro-4-vinyl-phenylamino)-phenyl]-[1,3,4]oxadiazol-2-yl}-(2-morpholin-4-yl-ethyl)amine: m. p. 174-177° C.; ¹H NMR (400 MHz, CDCl₃) δ 9.03 (s, 1 H), 7.48 (ddd, J=9.0, 5.5, 2.1 Hz, 1 H), 7.17 (dd, J=12.0, 1.9 Hz, 1 H), 7.07 (dd, J=8.3, 1.7 Hz, 1 H), 6.93 (td, J=8.3, 5.1 Hz, 1 H), 6.81 (dt, 7.1, 9.0 Hz, 1 H), 6.63 (dd, J=17.6, 11.0 Hz, 1 H), 5.65 (d, J=17.6 Hz, 1 H), 5.46 (t, J=5.0 Hz, 1 H), 5.21 (d, J=11.0 Hz, 1 H), 3.73 (apparent t, J=4.6 Hz, 4 H), 3.50 (dt, J=6.1, 5.3 Hz, 2 H), 2.65 (m, 2 H), 2.51 (apparent t, J=4.4 Hz, 4 H). Anal. Cald/Found for C₂₂H₂₂F₃N₅O₂: C, 59.32/59.11; H, 4.98/4.90; N, 15.72/15.50.

C26ELSA IC₅₀=0.71 μM

### EXAMPLE 157

### 2-(4-{2,3-difluoro-6-[5-(2-morpholin-4-yl-ethylamino)-[1,3,4]oxadiazol-2-yl]-phenylamino}-3-fluoro-phenyl)-ethanol

Step 1

To a stirring solution of {5-[3,4-Difluoro-2-(2-fluoro-4-iodo-phenylamino)-phenyl]-[1,3,4]oxadiazol-2-yl}-(2-morpholin-4-yl-ethyl)-amine (2.80 g, 5.13 mmol) and 2,4,6-Trivinyl-cyclotriboroxane pyridine complex (1.12 g, 4.65 mmol) in dimethoxyethane (30 mL) and water (10 mL) was added potassium carbonate (699 mg, 5.06 mmol) and tetrakis(triphenylphosphine)palladium (260 mg, 0.23 mmol). The reaction mixture was heated at reflux for 2 h, diluted with ethyl acetate (100 mL) and washed with water (2×20 mL) and saturated brine (2×20 mL). The organics were dried over magnesium sulfate, concentrated under reduced pressure, and purified by silica gel chromatography. Gradient elution with dichloromethane-15% methanol/dichloromethane afforded {5-[3,4-difluoro-2-(2-fluoro-4-vinyl-phenylamino)-phenyl]-[1,3,4]oxadiazol-2-yl}-(2-morphlin-4-yl-ethyl)-amine as a pale yellow foam (1.55 g) contaminated with ca. 0.15 mol % triphenylphosphine oxide and 10 mol % 2,4,6-Trivinyl-cyclotriboroxane pyridine complex.

Step 2

To a 0° C. solution of the impure product of step 1, {5-[3,4-difluoro-2-(2-fluoro-4-vinyl-phenylamino)-phenyl]-[1,3,4]oxadiazol-2-yl}-(2-morphlin-4-yl-ethyl)-amine (1.19 g, 2.67 mmol) in THF was added 1M borane-THF (9 mL, 9 mmol). Allowed reaction solution to warm to room temperature over 23 h. Added sodium borate monohydrate (3.0 g, 26.7 mmol) to the reaction solution. After stirring for 63 h at room temperature, water (6 mL) was added to the reaction solution. Filtered the mixture, rinsing the filter cake with water and ethyl acetate. Extracted the aqueous layer with ethyl acetate. Neutralized the aqueous layer to pH 7 and extracted with ethyl acetate. The extracts were combined and the solvent was removed in vacuo to obtain a foam. Chromatographed crude material on silica gel, eluting with a gradient of methylene chloride to 10% methanol in methylene chloride. Combined fractions and removed the solvent in vacuo to obtain a 0.874 g of a mixture containing triphenyphospine oxide and 2-(4-{2,3-difluoro-6-[5-(2-morpholin-4-yl-ethylamino)-[1,3,4]oxadiazol-2-yl]-phenylamino}-3-fluoro-phenyl)-ethanol and 1-(4-{2,3-difluoro-6-[5-(2-morpholin-4-yl-ethylamino)-[1,3,4]oxadiazol-2-yl]-phenylamino}-3-fluoro-phenyl)-ethanol.

Step 3

The mixture of 2-(4-{2,3-difluoro-6-[5-(2-morpholin-4-yl-ethylamino)-[1,3,4]oxadiazol-2-yl]-phenylamino}-3-fluoro-phenyl)-ethanol and 1-(4-{2,3-difluoro-6-[5-(2-morpholin-4-yl-ethylamino)-[1,3,4]oxadiazol-2-yl]-phenylamino}-3-fluoro-phenyl)-ethanol (contains triphenylphosphine) prepared in step two was dissolved in dimethylformamide, cooled to 0° C., and treated with imidazole (2 eq) and triisopropylsilylchloride (2 eq). The reaction mixture was allowed to warm to ambient temperature over 15 h. Partitioned reaction mixture between saturated aqueous sodium bicarbonate and ethyl acetate. Extracted the aqueous layer with ethyl acetate. Combined the ethyl acetate extracts and washed them with brine. The extracts were dried over magnesium sulfate, filtered and concentrated in vacuo to obtain an oil. Chromatographed crude oil on silica gel using a gradient of 5% methanol in methylene chloride to 10% methanol in methylene chloride over 40 min. Combined fractions and removed the solvent in vacuo. Further chromatographed the obtained oil on silica gel using 3% methanol in methylene chloride. Combined fractions and removed the solvent in vacuo to obtain a mixture of (5-{3,4-difluoro-2-[2-fluoro-4-(2-tripropylsilanyloxy-ethyl)-phenylamino]-phenyl}-[1,3,4]oxadiazol-2-yl)-2-morpholin-4-yl-ethyl)-amine and (5-{3,4-Difluoro-2-[2-fluoro-4-(1-tripropylsilanyloxy-ethyl)-phenylamino]-phenyl}-[1,3,4]oxadiazol-2-yl)-)2-morphlin-4-yl-ethyl)-amine.

Step 4

To a solution of (5-{3,4-difluoro-2-[2-fluoro-4-(2-tripropylsilanyloxy-ethyl)-phenylamino]-phenyl}-[1,3,4]oxadiazol-2-yl)-2-morpholin-4-yl-ethyl)-amine and (5-{3,4-Difluoro-2-[2-fluoro-4-(1-tripropylsilanyloxy-ethyl)-phenylamino]-phenyl}-[1,3,4]oxadiazol-2-yl)-)2-morphlin-4-yl-ethyl)-amine in methylene chloride was added triethylsilane (20 eq) and triflic acid (5 eq). After 64 h of stirring at room temperature, the solvent was removed in vacuo to obtain a residue. The residue was dissolved in ethyl acetate and washed with water and brine. Dried the ethyl acetate extracts over magnesium sulfate. Filtered the mixture and removed the ethyl acetate in vacuo to obtain an oil. Chromatographed the obtained oil on silica gel using 5% acetone in hexanes for 5 min., 40% acetone in hexanes for 36 min., a gradient of 60% acetone in hexanes to 80% acetone in hexanes over 10 min, 80% acetone in hexanes over 16 min. Combined fractions and removed the solvent in vacuo to obtain a mixture of 2-(4-(2,3-difluoro-6-[5-(2-morpholin-4-yl-ethylamino)-[1,3,4]oxadiazol-2-yl]-phenylamino}-3-fluoro-phenyl)-ethanol and 1-(4-{2,3-difluoro-6-[5-(2-morpholin-4-yl-ethylamino)-[1,3,4]oxadiazol-2-yl]-phenylamino}-3-fluoro-phenyl)-ethanol. Resubmitted the mixture to triethylsilane (20 eq) and triflic acid (5 eq). After 17 h of stirring the solvent was removed in vacuo. Chromatographed residue on silica gel eluting with 5% methanol in methylene chloride. Combined fractions and removed the solvent in vacuo to obtain a gum. Dried gum under vacuum at 80° C. for c.a. 60 h to isolate 2-(4-{2,3-difluoro-6-[5-(2-morpholin-4-yl-ethylamino)-[1,3,4]oxadiazol-2-yl]-phenylamino}-3-fluoro-phenyl)-ethanol as a solid: ¹H-NMR (CDCl₃, 400 Hz): δ 8.96 (1H, NH, br. s.), 7.45 (1H, ArH, m), 6.94 (3H, ArH, m), 6.76 (1H, ArH, m), 5.51 (1H, NH/OH, v. br. s.), 3.85 (2H, CH₂CH₂OH, t, J=6.34 Hz), 3.74 (4H, NCH₂, br. t., J=4.14 Hz), 3.50 (2H, HNCH₂CH₂, q, J=11.22, 5.42 Hz), 2.83 (2H, HNCH₂CH₂, t, J=6.58 Hz), 2.66 (2H, CH₂CH₂OH, br. t., J=5.369 Hz), 2.52 (4H, OCH₂, br. m.); ¹⁹F-NMR (CDCl₃, 376 Hz): δ−127.38 (1F, t, J=10.11 Hz), −133.10 (1F, t, J=0.027 Hz), −145.02 (1F, d, J=0.047); MS (APCI+)=464.4, MS(APCI−)=462.2. HPLC (Alitech Alltima C18, 3 μm column, 254 nM, mobile phase: 90/10 AcCN/water to 10/90 AcCN water in 10 minutes): 5.697 min, 96.15%. C26ELSA % Inhib@5 μM=33% C26ELSA % Inhib@0.5 μM=31%

### EXAMPLE 158

### 2-{5-[3,4-Difluoro-2-(2-fluoro-4-iodo-phenylamino)-phenyl]-[1,3,4]oxadiazol-2-ylsulfanyl}-ethanol

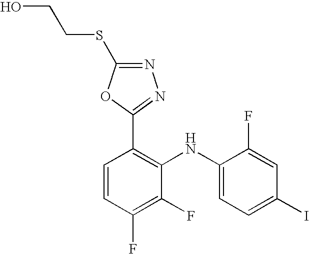

### EXAMPLE 159

### [6-(5-Allylsulfanyl-[1,3,4]oxadiazol-2-yl)-2,3-difluoro-phenyl]-(2-fluoro-4-iodo-phenyl)-amine

### EXAMPLE 160

### 2-{5-[3,4-Difluoro-2-(2-fluoro-4-methylsulfanyl-phenylamino)-phenyl]-[1,3,4]oxadiazol-2-ylamino}-propane-1,3-diol

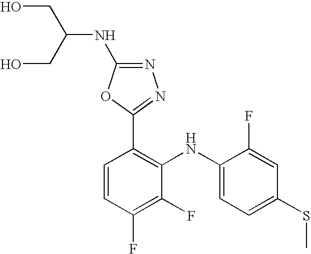

### EXAMPLE 161

### 2-{5-[2-(4-Ethyl-2-fluoro-phenylamino)-3,4-difluoro-phenyl]-[1,3,4]oxadiazol-2-ylamino}-propane-1,3-diol

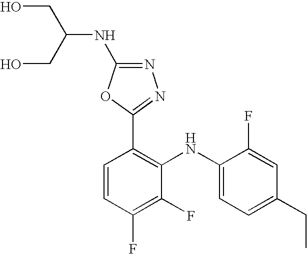

### EXAMPLE 162

### 3-{5-[3,4-Difluoro-2-(2-fluoro-4-methylsulfanyl-phenylamino)-phenyl]-[1,3,4]oxadiazol-2-ylamino}-propane-1,2(R)-diol

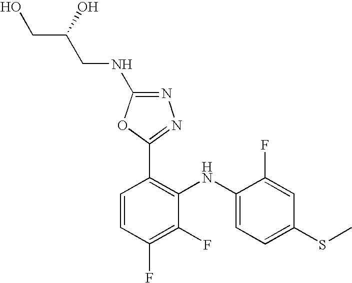

### EXAMPLE 163

### 3-{5-[3,4-Difluoro-2-(2-fluoro-4-methylsulfanyl-phenylamino)-phenyl}-[1,3,4]oxadiazol-2-ylamino}-propane-1,2(S)-diol

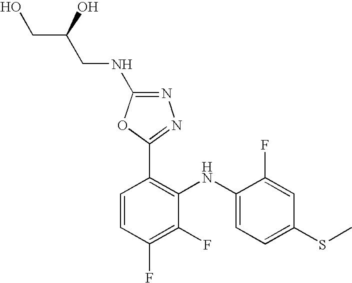

### EXAMPLE 164

### 3-{5-[2-(4-Ethyl-2-fluoro-phenylamino)-3,4-difluoro-phenyl]-[1,3,4]oxadiazol-2-ylamino}-propane-1,2(S)-diol

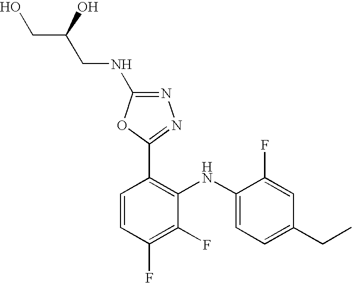

### EXAMPLE 165

### 3-{5-[2-(4-Ethynyl-2-fluoro-phenylamino)-3,4-difluoro-phenyl]-[1,3,4]oxadiazol-2-ylamino}-propane-1,2(S)-diol

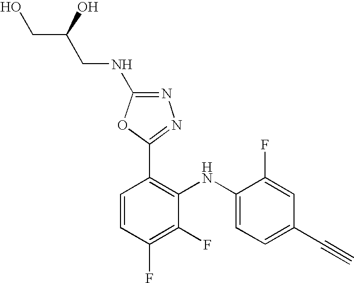

### EXAMPLE 166

### 2-{5-[3,4-Difluoro-2-(2-fluoro-4-methylsulfanyl-phenylamino)-phenyl]-[1,3,4]oxadiazol-2-ylamino}-ethanol

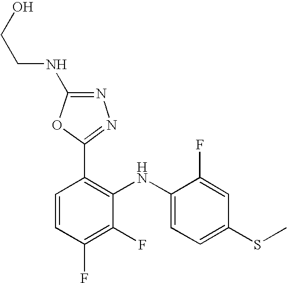

### EXAMPLE 167

### 2-(4-{2,3-difluoro-6-[5-(2-methylamino-ethylamino)-1[1,3,4]oxadiazol-2-yl]-phenylamino}-3-fluoro-phenyl)-ethanol

Step 1

Compound 6 from Example 18, (2-{5-[3,4-Difluoro-2-(2-fluoro-4-iodo-phenylamino)-phenyl]-[1,3,4]oxadiazol-2-ylamino}-ethyl)-methyl-carbamic acid tert-butyl ester, 2.71 g, 4.6 mmol) in dimethoxyethane (36 mL) was stirred under nitrogen with tetrakis(triphenylphosphine)palladium(0) (265 mg, 5%) for 20 minutes. At that time potassium carbonate (635 mg, 4.6 mmol) , water (12 mL), and vinylboronic acid pryidine complex (1.1 g, 4.6 mmol) was added. The solution was heated to reflux for 2 hours, and the reaction appeared complete by MS. After cooling to room temperature the reaction was poured into ethyl acetate washed with sodium bicarbonate solution, and dried over magnesium sulfate providing an orange oil. Chromatography on silica gel eluting with 20% ethyl acetate in dichloromethane provided product as a colorless foam (1.58 g, 70%).

Step 2

Compound from above (1.58 g, 3.22 mmol) in dry tetrahydrofuran at 0° C. was added borane-tetradydrofuran complex (2.0 equivalents). After stirring 1.5 hours, sodium perborate (1.92 g, 19.3 mmol) slurried in water (15 mL) was added and the reaction was stirred for 3 hours. The reaction mixture was poured into water, extracted with ethyl acetate and the organic phase was washed with brine and dried over magnesium sulfate to provide a yellow foam. Chromatography on silica gel eluting with 30% ethyl acetate in dichloromethane gave product as a colorless solid (1.09 g, 69%).

Step 3

Compound from above (1.09 g) in dichloromethane was treated with HCl gas. After standing for 1 hour and oily precipitate forms. The solution was evaporated and then triturated with methanol/acetonitrile and evaporated to give a light yellow solid (800 mg). 1H NMR (DMSO) 8.94(s, 1 H), 8.78(brs 2H), 8.25(t, 1H), 7.54(m, 1H), 7.17(m, 1H), 7.10(d, 1H), 6.93(s, 2H), 3.55(m, 4H), 3.12(m, 2H), 2.64(t, 1H), 2.55(t, 3H); Analysis calculated for C19H₂₀N₅O2F₃ corrects for 1.85 HCl, 0.80 water C 46.63/46.63, H 4.83/4.83, N 14.31/14.31.

C26CPA1 IC₅₀=0.91 μM

### EXAMPLE 168

### N-(2-{5-[2-(4-Ethyl-2-fluoro-phenylamino)-3,4-difluoro-phenyl]-[1,3,4]oxadiazol-2-ylamino}-ethyl)-N-methyl-acetamide

Step 1

N-{5-[3,4-Difluoro-2-(2-fluoro-4-iodo-phenylamino)-phenyl]-[1,3,4]oxadiazol-2-yl}-N′-methyl-ethane-1,2-diamine as a hydrochloride salt (1.0 g, 1.78 mmol) in dichloromethane (10 mL) was treated with triethylamine (792 uL, 5.7 mmol) and acetic anhydride (167 uL, 1.78 mmol). The reaction was stirred 16 hours at room temperature, diluted with additional solvent, and then washed with water and then sodium bicarbonate solution. To provide a colorless foam (712 mg, 76%) that was sufficiently pure for further use.

Step 2

Product from step 1 (712 mg, 1.3 mmol) in dimethoxyethane (9 mL) was treated with tetrakis(triphenylphosphine)palladium(0) (77 mg, 5%) for 30 minutes. At this time, potassium carbonate (185 mg, 1.3 mmol), vinyl boronic acid pryidine complex (322 mg, 1.3 mmol) and water (3 mL) was added and heated to reflux for 1.5 hours. At this time the reaction was judged complete, and was evaporated to dryness. The residue was chromatographed on silica gel eluting with 4% methanol in dichloromethane to give product as a colorless oil (450 mg, 80%) that was used directly in the next step.

Step 3

Product from step 3 (450 mg, 1.0 mmol) in tetrahydrofuran (16 mL) was treated with 5% Pd on C (50 mg) and place under 50 psi hydrogen pressure. When the pressure drop was complete, the catalyst was removed by filtration providing product as a colorless foam from dicholromethane (383 mg, 85%). 1H NMR (DMSO) 8.93(s, 1H), 7.42(m, 1H), 6.97-6.68(m, 4H), 6.06(t, 1H), 3.65(m, 2H), 3.58(m, 2H), 3.06(s, 3H), 2.59(q, 2H), 2.10(s, 3H), 1.20(t, 3H); Analysis corrects for 0.12 dicholromethane, C 57.27/57.07, 4.95/5.27, 15.76/15.37.

### EXAMPLE 169

### 2-(5-{3,4-difluoro-2-[2-fluoro-4-(2-hydroxy-ethyl)phenylamino]-phenyl}-[1,3,4]oxadiazol-2-ylamino)ethanol

Step 1

[2-(tert-butyl-dimethyl-silanyloxy)-ethyl]-{5-[3,4-difluoro-2-(2-fluoro-4-iodo-phenylamino)-phenyl]-[1,3,4]oxadiazol-2-yl}-amine (1.26 g, 2.1 mmol in dimethoxyethane (18 mL) was treated with tetrakis(triphenylphosphine)palladium(0) (123 mg, 5%) for 30 minutes. At this time, potassium carbonate (295 mg, 2.1 mmol), vinyl boronic acid pryidine complex (513 mg, 2.1 mmol) and water (6 mL) was added and heated to reflux for 1.5 hours. At this time the reaction was judged complete, cooled to room temperature and poured into water. Product was extracted with ethyl acetate, the organic layer was washed with sodium bicarbonate solution and then brine. After drying over magnesium sulfate the solvent was removed under reduced pressure to give an oil. Chromatography in silica gel 4:1 hexane/ethyl acetate provided a light yellow oil that solidified upon standing (780 mg, 75%).

Step 2

Compound from above (780 mg, 1.6 mmol) in dry tetrahydrofuran at 0° C. was added borane-tetradydrofuran complex (2.0 equivalents). After stirring 1.5 hours, sodium perborate (952 mg, 9.6 mmol) slurried in water (10 mL) was added and the reaction was stirred for 3 hours. The reaction mixture was poured into water, extracted with ethyl acetate and the organic phase was washed with brine and dried over magnesium sulfate to provide a yellow foam. Chromatography on silica gel eluting with 25% ethyl acetate in dichloromethane gave product as a colorless solid (448 mg, 60%).

Step 3

Compound from above (448 mg, 0.88 mmol) in tetrahydrofuran (10 mL) was treated with acetic acid (50 uL, 0.88 mmol) and then tetrabutylammonium fluoride (1.3 mL of 1.0M solution in tetrahydrofuran) for 5 hours. The reaction was poured into ethylacetate, washed with water and then brine. After drying over magnesium sulfate the solvent was evaporated to give a colorless foam. Trituration of the foam with 1:1 dichloromethane/hexane provided a colorless solid that was collected by filtration (275 mg, 79%). 1HNMR (DMSO) 8.83(s, 1H), 7.95(t, 1H), 7.52(m, 1H), 7.15(m, 1H), 7.10(d, 1H), 6.93(s, 2H), 4.75(t, 1H), 4.61(t, 1H), 3.55(m, 4H), 3.24(m, 2H), 2.67(t, 2H). Analysis corrects for 0.59 water C53.08/53.38, H 4.13/4.52, 13.61/13.84. C26ELSA IC₅₀=5.1 μM

### EXAMPLE 170

### Acetic Acid 2-{5-[2-(4-ethyl-2-fluoro-phenylamino)-3,4-difluoro-phenyl]-[1,3,4]oxadiazol-2-ylamino]-ethyl Ester

T a solution comprised of 2-{5-[2-(4-ethyl-2-fluoro-phenylamino)-3,4-difluoro-phenyl]-[1,3,4]oxadiazol-2-ylamino}-ethanol (0.25 g), pyridine, acetic anhydride, and dichloromethane (5 mL) was added several grains of 4-dimethylaminopyridine. The reaction mixture was stirred under an argon atmosphere for 44 minutes. The reaction mixture was quenced with 10% aqueous hydrochloric acid. The mixture was then diluted with dichloromethane (350 mL) and water (50 mL). The organic phase was separated and dried over magnesium sulfate. Thin layer chromatography analysis of the dried dichloromethane filtrate showed a single product spot (R_{f}=0.61, 9:1 v/v dichloromethane:methanol; UV detection: R_{f} for starting material=0.41). The filtrate was concentrated under reduced pressure (40° C.) to give a clear viscous film. Reconstitution in dichloromethane, transfer to a reaction vial, and reconcentration under reduced pressure gave a white amorphous solid. This crude product was dissolved in absolute ethanol (5 mL) over a steam bath, and the solution was boiled down to minimal volume (˜2 mL) and was cooled over an ice bath for about one hour. No crystallization ensued. Hexanes (˜3 mL) were added to the ethanol solution until slightly turbid. Within about one minute, small white crystals began to form suspended in solution and adhered to the insides of the vessel. The precipitate was collected by vacuum filtration and was dried with suction to afford the white microneedles (0.1459 g).

As used herein, the terms “patient” or “recipient” refer to any warm-blooded animal, preferably a mammal such as, but not limited to, a human, horse, dog, cat, guinea pig, or mouse. Preferably, the patient is human.

The terms “treat”, “treating”, or “treatment” for purposes of the present invention refer to delay of onset, prophylaxis or prevention, amelioration, inhibition, or elimination of a named condition, or the diminution of its physiological symptoms or manifestations, in a patient or recipient once the condition has been established. A therapeutically or pharmaceutically effective amount of a compound of this invention or other pharmaceutically useful agent will be understood to be an amount of the compound or compounds in question which will bring about the before mentioned delay of onset, prophylaxis or prevention, amelioration, inhibition, or elimination of a named condition, or the diminution of its physiological symptoms or manifestations.

Selective MEK 1 or MEK 2 inhibitors are those compounds that inhibit the MEK 1 or MEK 2 enzymes, respectively, without substantially inhibiting other enzymes such as MKK3, PKC, Cdk2A, phosphorylase kinase, EGF, and PDGF receptor kinases, and C-src. In general, a selective MEK 1 or MEK 2 inhibitor has an IC₅₀ for MEK 1 or MEK 2 that is at least one-fiftieth (1/50) that of its IC₅₀ for one of the above-named other enzymes. Preferably, a selective inhibitor has an IC₅₀ that is at least 1/100, more preferably 1/500, and even more preferably 1/1000, 1/5000, or less than that of its IC₅₀ or one or more of the above-named enzymes.

The disclosed compositions are useful as both prophylactic and therapeutic treatments for diseases or conditions related to the hyperactivity of MEK, as well as diseases or conditions modulated by the MEK cascade. Examples include, but are not limited to, stroke, septic shock, heart failure, osteoarthritis, rheumatoid arthritis, organ transplant rejection, and a variety of tumors such as ovarian, lung, pancreatic, brain, prostatic, and colorectal.

The invention further relates to a method for treating proliferative diseases, such as cancer, restenosis, psoriasis, autoimmune disease, and atherosclerosis. Other aspects of the invention include methods for treating MEK-related (including ras-related) cancers, whether solid or hematopoietic. Examples of cancers include brain, breast, lung, such as non-small cell lung, ovarian, pancreatic, prostate, renal, colorectal, cervical, acute leukemia, and gastric cancer. Further aspects of the invention include methods for treating or reducing the symptoms of xenograft (cell(s), skin, limb, organ or bone marrow transplant) rejection, osteoarthritis, rheumatoid arthritis, cystic fibrosis, complications of diabetes (including diabetic retinopathy and diabetic nephropathy), hepatomegaly, cardiomegaly, stroke (such as acute focal ischemic stroke and global cerebral ischemia), heart failure, septic shock, asthma, Alzheimer's disease, and chronic or neuropathic pain. Compounds of the invention are also useful as antiviral agents for treating viral infections such as HIV, hepatitis (B) virus (HBV), human papilloma virus (HPV), cytomegalovirus (CMV), and Epstein-Barr virus (EBV). These methods include the step of administering to a patient in need of such treatment, or suffering from such a disease or condition, a therapeutically effective amount of a disclosed compound or pharmaceutical composition thereof.

The term “chronic pain” for purposes of the present invention includes, but is not limited to, neuropathic pain, idiopathic pain, and pain associated with chronic alcoholism, vitamin deficiency, uremia, or hypothyroidism. Chronic pain is associated with numerous conditions including, but not limited to, inflammation, arthritis, and post-operative pain.

As used herein, the term “neuropathic pain” is associated with numerous conditions which include, but are not limited to, inflammation, postoperative pain, phantom limb pain, burn pain, gout, trigeminal neuralgia, acute herpetic and postherpetic pain, causalgia, diabetic neuropathy, plexus avulsion, neuroma, vasculitis, viral infection (including herpes viral infection, varicella zoster infection, and HIV infection), crush injury, constriction injury, tissue injury, limb amputation, arthritis pain, hypothyroidism, uremia, chronic alcoholism, post-operative pain, arthritis, back pain, and vitamin deficiencies and nerve injury between the peripheral nervous system and the central nervous system.

The invention also features methods of combination therapy, such as a method for treating cancer, wherein the method further includes providing radiation therapy or chemotherapy, for example, with mitotic inhibitors such as a taxane or a vinca alkaloid. Examples of mitotic inhibitors include paclitaxel, docetaxel, vincristine, vinblastine, vinorelbine, and vinflunine. Other therapeutic combinations include a MEK inhibitor of the invention and an anticancer agent such as cisplatin, 5-fluorouracil or 5-fluoro-2-4(1H,3H)-pyrimidinedione (5FU), flutamide, and gemcitabine.

The chemotherapy or radiation therapy may be administered before, concurrently, or after the administration of a disclosed compound according to the needs of the patient.

Cancers which may be inhibited, treated or controlled with the compounds, methods and pharmaceutical formulations herein include, but are not limited to, cancers of the breast, prostate, testicular, lung, ovarian, uterine, kidney, bladder, colon, rectum, stomach, pancreatic, hepatic, melanoma, esophageal, brain, Kaposi's sarcoma, squamous cell carcinomas, oral carcinomas, leukemias, gliomas and lymphomas.

A further embodiment of this invention is a method of treating subjects suffering from diseases caused by cellular proliferation. The method entails inhibiting proliferation of tumorigenic cells of epithelial origin and vascular smooth muscle proliferation, and/or cellular migration by administering a therapeutically effective amount of a compound of this invention to a subject in need of treatment.

A further embodiment of this invention is a method of treating subjects suffering from diseases caused by DNA tumor viruses such as herpes viruses.

The compounds of this invention may also be used in therapeutic combinations with inhibitors of cyclin-dependent kinases (CDK). These include synthetic CDK inhibitors, such as purines, alkaloids, indirubins, flavonoids, paullones, butyrolactone I and hymenialdisine.

Examples of purines which may be used in pharmaceutical combinations and regimens of this invention include olomoucine, roscovitine, CVT-313, isopentyl-adenine, purvalanol B and 6-Cyclohexylmethoxy-9H-purin-2-ylamine, also known as NU-2058. Useful alkaloid CDK inhibitors include staurosporine, UCN-01 and CPG 41 251. Indirubins include indirubin and its analogues, including indirubin-5-sulphonic acid, 5-chloro-indirubin and indirubin-3′-monoxime. Useful Flavonoids include flavopiridol, its deschloro derivative, L86-8276, and thioflavopiridol. Also useful is genistein, a naturally occurring isoflavone.

The compounds herein may also be used in drug regimens with taxanes, such as paclitaxel and docetaxel.

For indications in the treatment of bladder cancer, the compounds of this invention may be used in regimens with agents such as PACIS® (BCG,live—BioChem Pharma Inc.) and VALSTAR® (valrubicin—Anthra Pharmaceuticals). Brain cancer, including recurrent glioblastoma multiforme, combinations may include GLIADEL® (carmustine wafer for implantation), sponsored by Guilford Pharmaceuticals Incorporated.

Breast cancer drugs which may be used in combinations of this invention include ADRIAMYCIN® (doxorubicin), AREDIA® (pamidronate disodium for injection—Ciba Geigy Corporation Pharmaceuticals Division), ARIMIDEX® (anastrozole—AstraZeneca Pharmaceuticals), AROMASIN® (exemestane—Pharmacia & Upjohn Company, CYTOXAN® (cyclophosphamide), ELLENCE® (epirubicin hydrochloride—Pharmacia & Upjohn), FARESTON® (toremifene citrate—Orion Corporation), FEMARA® (letrozole—Novartis Pharmaceuticals Company), GEMZAR® (gemcitabine), HERCEPTIN® (trastuzumab—Genentech, Inc.), MEGACE® (megestrol), NAVELBINE® (vinorelbine), NOLVADEX® (tamoxifen citrate—AstraZeneca Pharmaceuticals), TAXOL® (paclitaxel—Bristol-Myers Squibb), TAXOTERE® (docetaxel—Aventis, Inc.), XELODA® (capecitabine—Roche), and ZOLADEX® (goserelin acetate).

The compounds of this invention can also be used in advance of, in combination with, or following chemotherapy combinations or regimens known in the art. Examples of chemotherapy combinations utilized in treatment or inhibition of breast cancer include cyclophosphamide (CYTOXAN®), methotrexate (AMETHOPTERIN®, MEXATE®, or FOLEX®), and fluorouracil (Fluorouracil, 5-Fu, OR ADRUCIL®). This combination therapy is often called “CMF”. Another related regimen is the administration of doxorubicin (ADRIAMYCIN®), followed by the “CMF” therapy. The regimen referred to as “CAF” comprises combinations of cyclophosphamide, doxorubicin, and fluorouracil. Combinations of doxorubicin (ADRIAMYCIN®) and cyclophosphamide are called “AC”. Another conventional therapeutic breast cancer combination is the AC regiment, doxorubicin (ADRIAMYCIN®) and cyclophosphamide, combined with paclitaxel (TAXOL®). Another conventional regimen of treatment is the combination of cyclophosphamide (CYTOXAN®), epirubicin (ELLENCE®) and fluorouracil.

Combination therapies for colon and rectal cancer may include an effective amount of a compound of this invention and CAMPTOSAR® (irinotecan hydrochloride) injection, available from Pharmacia & Upjohn.

Head and neck cancers, including moderate to severe xerostomia, may be treated with a compound of this invention and ETHYOL® (amifostine) for Injection, available from US Bioscience. Regimens for treatment or amelioration of Kaposi's Sarcoma include compounds of this invention and PANRETIN® (Alitretinoin gel 0.1%—Ligand Pharmaceuticals), DAUNOXOME® (daunorubicin citrate liposome—NeXstar), TAXOL® (paclitaxel for Injection—Bristol Myers Squibb Co. Pharmaceutical Research Institute).

Leukemia regimens can include combinations with BUSULFEX® (busulfan—Orphan Medical Inc), CAMPATH® (alemtuzumab—from Millennium and ILEX Partners, LP) Daunorubicin HCL (Bedford Laboratories, Div. Ben Venue Laboratories, Inc.), Elliotts B Solution (calcium chloride, dextrose, magnesium sulfate, potassium chloride, sodium bicarbonate, sodium chloride, sodium phosphate, dibasic) for injection, sponsored by Orphan Medical Incorporated, GLEEVEC® (imatinib mesylate) from Novartis Pharmaceuticals Corporation, NEUPOGEN® (filgrastim) by Amgen, Inc., MYLOTARG® (gemtuzumab ozogamicin) for injection, available from Wyeth, and TRISENOX® (arsenic trioxide) from Cell Therapeutics, Inc.

Lung cancer regimens include combinations of agents of the present invention and ETHYOL® (amifostine—Alza), ETOPOPHOS® (etoposide phosphate—Bristol-Myers Squibb), GEMZAR® (gemcitabine HCL for injection—Eli Lilly & Co.) HYCAMTIN® (topotecan hydrochloride for injection—GlaxoSmithKline), TAXOL® (paclitaxel for Injection—Bristol Myers Squibb Co. Pharmaceutical Research Institute), TAXOTERE® (docetaxel—available from Aventis Pharmaceuticals).

Combination treatments for lymphoma, such as meningeal leukemia or lymphocytic lymphoma, may be include Elliotts B Solution (calcium chloride, dextrose, magnesium sulfate, potassium chloride, sodium bicarbonate, sodium chloride, sodium phosphate, dibasic for injection—Orphan Medical Incorporated) in mixes with methotrexate sodium and/or cytarabine for intrathecal administration. Also useful are Intron A (interferon alfa-2a—Schering Corp.), RITUXAN® (rituximab) sponsored by Genentech, Inc., ONTAK® (denileukin diftitox), marketed by Ligand Pharmaceuticals and manufactured by Seragen, Inc., for the treatment of persistent or recurrent cutaneous t-cell lymphoma, (CTCL), a rare slow-growing form of non-Hodgkin's lymphoma, in which malignant cells express the CD25 component of the IL-2 receptor. The compounds of this invention may also be used in regimens with TARGRETIN® (bexarotene) capsules, from Ligand Pharmaceuticals Inc., for treatment of cutaneous manifestations of cutaneous T-cell lymphoma, particularly in patients who are refractory to at least one prior systemic therapy, or with UVADEX® (methoxsalen sterile solution, 20 mcg/mL), available from Therakos, Inc., for palliative treatment of skin manifestations of cutaneous T-cell lymphoma that have been unresponsive to other treatments.

In combinations for the treatment or inhibition of melonoma the compounds may be combined in regimens with PROLEUKIN® (aldesleukin) from Chiron Corporation, particularly for treatment of adults with metastatic melanoma and for metastatic renal cell carcinoma patients.

The compounds herein may be used in regimens with DepoCyt® (cytarabine liposomal injection, 10 mg/mL), by DepoTech Corporation, for treatment of lymphomatous meningitis or other forms of neoplastic meningitis associated with solid tumors, lymphoma or leukemia.

DOSTINEX® (cabergoline) Tablets, from Pharmacia & Upjohn Company, may be combined with compounds herein for the treatment of hyperprolactinemic disorders, either idiopathic or due to pituitary adenomas.

For inhibition or treatment of ovarian cancers, the compound herein may be combined with DOXIL® (doxorubicin HCL liposome injection), from Alza Corporation, HYCAMTIN® (topotecan HCL), from SmithKline Beecham, or TAXOL® (paclitaxel) from Bristol-Myers Squibb Company.

In regimens for pancreatic cancers, combinations herein may include GEMZAR® (gemcitabine HCL), available from Eli Lilly & Co. For prostate cancers, combination can include LUPRON DEPOT® (leuprolide acetate) for Injection, sponsored by TAP Holdings Incorporated, NILANDRON® (nilutamide) Tablets, sponsored by GH Besselaar Associates Incorporated, NOVANTRONE® (mitoxantrone hydrochloride) for Injection, Immunex Corporation, TRELSTAR DEPOT® (triptorelin palmoate) for injectable suspension, from Debio Recherche Pharmaceutique S.A., VIADUR® (leuprolide acetate implant), from Alza Corporation, ZOLADEX® (goserelin acetate implant) by Zeneca Pharmaceuticals, or the Urowave Microwave Thermotherapy System by Dornier Medical Systems, Inc., which is a non-surgical treatment alternative to transurethral resection of the prostate.

The compounds of this invention may also be used prior to, in conjunction with or following regimens of chemotherapeutic alkylating agents. Useful alkylating agents include those known in the art including bis(chlorophenyl)amines such as cyclophosphamide, mechloroethamine, chlorambucil or melphalan; nitrosureas such as carmustine, lomustine or semustine; aziridines such as thiotepa or triethylenemelamine; alkylsulfonates, such as busulfan; or other alkylation agents, including procarbazine, dacarbazine, hexamethylmelamine and cisplatin.

The compounds of this invention may also be used in pharmaceutical combinations and regimens and other treatment methods for restinosis. The compounds herein may be used with brachytherapy (gamma or beta radiation), sonotherapy, cryotherapy, endothelial cell implantations or nitric oxide treatments for restinosis. They may also be administered in conjunction with vascular stents used following angioplasty, including biodegradable stents, and drug-coated or other drug-eluting or DNA-coated stents. Examples of compounds which may be used in drug-containing stents include dexamethasone, Actinomycin-D, rapamycin, sirolimus or paclitaxel.

Anti-platelet drugs which may be used along with compounds of this invention in treating, inhibiting or delaying onset of restinosis, optionally along with drug-eluding stents, are the platelet glycoprotein Ilb/IIIa inhibitors, such as abciximab, eptifabatide, Integrelin, lamifiban and tirofiban. Other useful anti-platelet agents include aspirin, cilostazol, ticlopidine, clopdogrel, sulfinpyrazone, dipyridamole, and Ridogrel.

Those skilled in the art will be able to determine, according to known methods, the appropriate pharmaceutically and therapeutically effective amount or dosage of a compound of the present invention to administer to a patient, taking into account factors such as age, weight, general health, the compound administered, the route of administration, the type of pain or condition requiring treatment, and the presence of other medications. In general, a pharmaceutically or a therapeutically-effective amount will be between about 0.1 and about 1000 mg/kg per day, preferably between about 1 and about 300 mg/kg body weight, and daily dosages will be between about 10 and about 5000 mg for an adult subject of normal weight. Commercially available capsules or other formulations (such as liquids and film-coated tablets) of 100, 200, 300, or 400 mg can be administered according to the disclosed methods.

The compounds of the present invention are preferably formulated prior to administration. Therefore, another aspect of the present invention is a pharmaceutical composition comprising a compound of Formula I and a pharmaceutically acceptable carrier. In making the compositions of the present invention, the active ingredient, such as a compound of Formula I, will usually be mixed with a carrier, or diluted by a carrier or enclosed within a carrier. Dosage unit forms or pharmaceutical compositions include tablets, capsules, pills, powders, granules, aqueous and nonaqueous oral solutions and suspensions, and parenteral solutions packaged in containers adapted for subdivision into individual doses.

Dosage unit forms can be adapted for various methods of administration, including controlled release formulations, such as subcutaneous implants. Administration methods include oral, rectal, parenteral (intravenous, intramuscular, subcutaneous), intracisternal, intravaginal, intraperitoneal, intravesical, local (drops, powders, ointments, gels, or cream), and by inhalation (a buccal or nasal spray).

Parenteral formulations include pharmaceutically acceptable aqueous or nonaqueous solutions, dispersion, suspensions, emulsions, and sterile powders for the preparation thereof. Examples of carriers include water, ethanol, polyols (propylene glycol, polyethylene glycol), vegetable oils, and injectable organic esters such as ethyl oleate. Fluidity can be maintained by the use of a coating such as lecithin, a surfactant, or maintaining appropriate particle size. Carriers for solid dosage forms include (a) fillers or extenders, (b) binders, (c) humectants, (d) disintegrating agents, (e) solution retarders, (f) absorption accelerators, (g) adsorbents, (h) lubricants, (i) buffering agents, and (j) propellants. Compositions may also contain adjuvants such as preserving, wetting, emulsifying, and dispensing agents; antimicrobial agents such as parabens, chlorobutanol, phenol, and sorbic acid; isotonic agents such as a sugar or sodium chloride; absorption-prolonging agents such as aluminum monostearate and gelatin; and absorption-enhancing agents.

1. A compound of the formula: 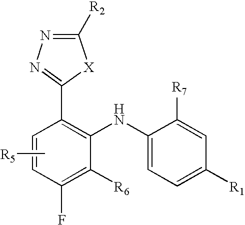
wherein:
X is NH, O or S;
R₁ is selected from halogen, —C═CH₂, —O—C₁-C₆ alkyl, —C═CH—(CH₂)ₘ—O—C₁-C₆ alkyl, C═C—(CH₂)ₘ—O—C₁-C₆ alkyl, —C═C—(CH₂)ₘ—NH—C₁-C₆ alkyl, —C≡CH, —C≡C—(CH₂)ₘ—OH, C≡C—(CH₂)ₘ—NH₂,—COOH, —(CH₂)ₘ—COOH, —CONH₂, —COO—C₁-C₆ alkyl, —(CH₂)ₘ—CONH₂, —C(O)(C₁-C₆ alkyl), —C(O)NHSO₂H, (CH₂)ₘ—C(O)NHSO₂H, —C(O)NHSO₂—(C₁-C₃ alkyl), —(CH₂)ₘ—C(O)NHSO₂—(C₁-C₃ alkyl), or C₁-C₆ alkyl, —S—C₁-C₆ alkyl or alkenyl, with the alkyl, —S-alkyl and alkenyl chains in each of these R₁ moieties being optionally substituted by from 1 to 3 OH groups and/or by from 1 to 5 fluorine atoms;
R₂ is —R₃, —O—R₃, —S—R₃ or a moiety selected from the group of NH₂, NHR₃, N(C₁-C₃ alkyl)-R₃, —NH—(CH₂)ₙ—O—(C₁-C₃ alkyl), —NH—(CH₂)ₙ—O—C(O)—(C₁-C₃ alkyl) or —N((CH₂)ₙ—O—(C₁-C₃ alkyl))₂, or a moiety selected from the group of: 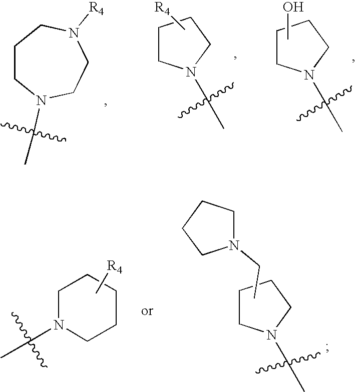
R₃ is a moiety selected from:
a) C₁-C₈ alkyl, C₁-C₈ alkenyl, the alkyl and alkenyl groups each being optionally substituted by from 1 to 4 OH or halogen groups;
b) —(CH₂)ₙ—NH₂, —(CH₂)ₙ—NH(C₁-C₃ alkyl), —(CH₂)ₙ—N(C₁-C₃ alkyl)₂, —(CH₂)ₙ—O—(C₁-C₃ alkyl), —(CH₂)ₘ—C(O)—O—(C₁-C₃ alkyl), or (CH₂)ₙ—N(C₁-C₃ alkyl)-C(O)—(C₁-C₃ alkyl);
c) phenyl, —(CH₂)ₘ-phenyl, —(CH₂)ₘ—O-phenyl, the phenyl ring of each being optionally substituted by 1 or 2 groups selected from C₁-C₃ alkoxy, NH₂, NH(C₁-C₃ alkyl), N(C₁-C₃ alkyl)₂; or
d) a moiety selected from the group of: 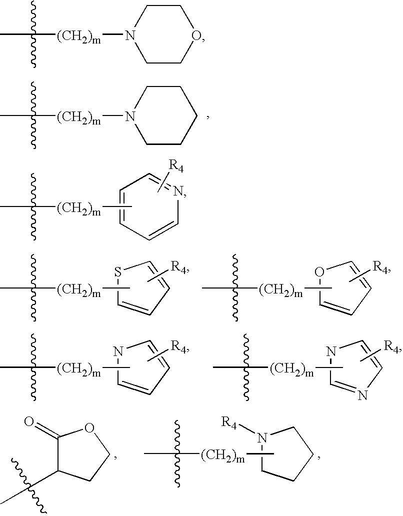
R₄ is selected from H or C₁-C₃ alkyl, optionally substituted by OH;
R₅ is selected from H or halogen;
R₆ is selected from H or F;
R₇ is selected from F, CH₂F, CHF₂, or —CF₃;
n in each instance is independently selected as an integer of from 1 to 6
m in each instance is independently selected as an integer of from 1 to 4;
or a pharmaceutically acceptable salt or ester form thereof.

2. A compound of claim 1 of the formula: 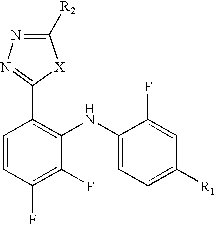
wherein:
R₁ is selected from I, Br or C₁-C₃ alkyl;
R₂ is R₃, —O—R₃, —S—R₃ or a moiety selected from the group of NH₂, NHR₃, N(C₁-C₃ alkyl)-R₃, —NH—(CH₂)ₙ—O—(C₁-C₃ alkyl), —NH—(CH₂)ₙ—O—C(O)—(C₁-C₃ alkyl) or —N((CH₂)ₙ—O—(C₁-C₃ alkyl))₂;
R₃ is a moiety selected from:
a) C₁-C₈ alkyl, C₁-C₈ alkenyl, the alkyl and alkenyl groups each being optionally substituted by from 1 to 4 OH groups;
b) —(CH₂)ₙ—NH₂, —(CH₂)ₙ—NH(C₁-C₃ alkyl), —(CH₂)ₙ—N(C₁-C₃ alkyl)₂, —(CH₂)ₙ—O—(C₁-C₃ alkyl), —(CH₂)ₙ—C(O)—O—(C₁-C₃ alkyl),
c) phenyl, —(CH₂)ₘ-phenyl, —(CH₂)ₘ—O-phenyl, the phenyl ring of each being optionally substituted by 1 or 2 groups selected from C₁-C₃ alkoxy, NH₂, NH(C₁-C₃ alkyl), N(C₁-C₃ alkyl)₂; or
d) a moiety selected from the group of: 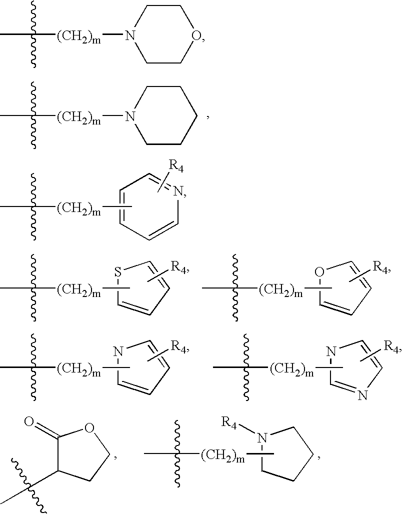
R₄ is selected from H or C₁-C₃ alkyl, optionally substituted by OH;
n is an integer of from 1 to 6
m is an integer of from 1 to 4;
or a pharmaceutically acceptable salt form thereof.

3. A compound of claim I selected from the group of:
5-[3,4-Difluoro-2-(2-fluoro-4-iodo-phenylamino)-phenyl]-[1,3,4]oxadiazol-2-ylamine;
5-[5-Chloro-3,4-difluoro-2-(2-fluoro-4-iodo-phenylamino)-phenyl]-[1,3,4]oxadiazol-2-ylamine;
5-[2-(4-Bromo-2-fluoro-phenylamino)-3,4-difluoro-phenyl]-[1,3,4]oxadiazol-2-ylamine;
{5-[3,4-Difluoro-2-(2-fluoro-4-iodo-phenylamino)-phenyl]-[1,3,4]oxadiazol-2-yl}-ethyl-amine;
Allyl-{5-[3,4-difluoro-2-(2-fluoro-4-iodo-phenylamino)-phenyl]-[1,3,4]oxadiazol-2-yl}-amine;
{5-[3,4-Difluoro-2-(2-fluoro-4-iodo-phenylamino)-phenyl]-[1,3,4]oxadiazol-2-yl}-(2,4-dimethoxy-phenyl)-amine;
N-{5-[3,4-Difluoro-2-(2-fluoro-4-iodo-phenylamino)-phenyl]-[1,3,4]oxadiazol-2-yl}-N′,N′-dimethyl-benzene-1,4-diamine;
3-{5-[3,4-Difluoro-2-(2-fluoro-4-iodo-phenylamino)-phenyl]-[1,3,4]oxadiazol-2-ylamino}-propane-1,2-diol;
{5-[3,4-Difluoro-2-(2-fluoro-4-iodo-phenylamino)-phenyl]-[1,3,4]oxadiazol-2-yl}-(3-morpholin-4-yl-propyl)-amine hydrochloride salt;
N-{5-[3,4-Difluoro-2-(2-fluoro-4-iodo-phenylamino)-phenyl]-[1,3,4]oxadiazol-2-yl}-N′,N′-diethyl-propane-1,3-diamine;
or a pharmaceutically acceptable salt form thereof.

4. A compound of claim 1 selected from the group of:
N′1′-{5-[3,4-Difluoro-2-(2-fluoro-4-iodo-phenylamino)-phenyl]-[1,3,4]oxadiazol-2-yl}-ethane-1,2-diamine as a trifluoro-acetic acid;
N-{5-[3,4-Difluoro-2-(2-fluoro-4-iodo-phenylamino)-phenyl]-[1,3,4]oxadiazol-2-yl}-N,N′,N′-trimethyl-propane-1,3-diamine;
{5-[3,4-Difluoro-2-(2-fluoro-4-iodo-phenylamino)-phenyl]-[1,3,4]oxadiazol-2-yl}-(2-piperidin-1-yl-ethyl)-amine;
N′1′-{5-[3,4-Difluoro-2-(2-fluoro-4-iodo-phenylamino)-phenyl]-[1,3,4]oxadiazol-2-yl}-butane-1,4-diamine;
N′1′-{5-[3,4-Difluoro-2-(2-fluoro-4-iodo-phenylamino)-phenyl]-[1,3,4]oxadiazol-2-yl}-propane-1,3-diamine;
N-{5-[3,4-Difluoro-2-(2-fluoro-4-iodo-phenylamino)-phenyl]-[1,3,4]oxadiazol-2-yl}-N′,N′-dimethyl-propane-1,3-diamine;
N-{5-[3,4-Difluoro-2-(2-fluoro-4-iodo-phenylamino)-phenyl]-[1,3,4]oxadiazol-2-yl}-N′,N′-dimethyl-ethane-1,2-diamine;
N-{5-[3,4-Difluoro-2-(2-fluoro-4-iodo-phenylamino)-phenyl]-[1,3,4]oxadiazol-2-yl}-N′-methyl-ethane-1,2-diamine;
N-{5-[3,4-Difluoro-2-(2-fluoro-4-iodo-phenylamino)-phenyl]-[1,3,4]oxadiazol-2-yl}-2,2,N′,N′-tetramethyl-propane-1,3-diamine;
{5-[3,4-Difluoro-2-(2-fluoro-4-iodo-phenylamino)-phenyl]-[1,3,4]oxadiazol-2-yl}-(3-methoxy-propyl)-amine;
or a pharmaceutically acceptable salt form thereof.

5. A compound of claim 1 selected from the group of:
{5-[3,4-Difluoro-2-(2-fluoro-4-iodo-phenylamino)-phenyl]-[1,3,4]oxadiazol-2-yl}-(2-morpholin-4-yl-ethyl)-amine;
(2,3-Difluoro-6-{5-[4-(2-methoxy-ethyl)-piperazin-1-yl]-[1,3,4]oxadiazol-2-yl}-phenyl)-(2-fluoro-4-iodo-phenyl)-amine;
5-{5-[3,4-Difluoro-2-(2-fluoro-4-iodo-phenylamino)-phenyl]-[1,3,4]oxadiazol-2-ylamino}-2,2-dimethyl-pentan-1-ol;
1-{5-[3,4-Difluoro-2-(2-fluoro-4-iodo-phenylamino)-phenyl]-[1,3,4]oxadiazol-2-yl}-pyrrolidin-3-ol;
(1-{5-[3,4-Difluoro-2-(2-fluoro-4-iodo-phenylamino)-phenyl]-[1,3,4]oxadiazol-2-yl}-piperidin-4-yl)-methanol;
2-(1-{5-[3,4-Difluoro-2-(2-fluoro-4-iodo-phenylamino)-phenyl]-[1,3,4]oxadiazol-2-yl}-piperidin-4-yl)-ethanol;
4-({5-[3,4-Difluoro-2-(2-fluoro-4-iodo-phenylamino)-phenyl]-[1,3,4]oxadiazol-2-yl}-ethyl-amino)-butan-1-ol;
1-{5-[3,4-Difluoro-2-(2-fluoro-4-iodo-phenylamino)-phenyl]-[1,3,4]oxadiazol-2-yl}-piperidin-4-ol
2-{5-[3,4-Difluoro-2-(2-fluoro-4-iodo-phenylamino)-phenyl]-[1,3,4]oxadiazol-2-ylamino}-ethanol;
3-{5-[3,4-Difluoro-2-(2-fluoro-4-iodo-phenylamino)-phenyl]-[1,3,4]oxadiazol-2-ylamino}-propan-1-ol;
or a pharmaceutically acceptable salt form thereof.

6. A compound of claim 1 selected from the group of:
4-{5-[3,4-Difluoro-2-(2-fluoro-4-iodo-phenylamino)-phenyl]-[1,3,4]oxadiazol-2-ylamino}-butan-1-ol;
5-{5-[3,4-Difluoro-2-(2-fluoro-4-iodo-phenylamino)-phenyl]-[1,3,4]oxadiazol-2-ylamino}-pentan-1-ol;
{5-[3,4-Difluoro-2-(2-fluoro-4-iodo-phenylamino)-phenyl]-[1,3,4]oxadiazol-2-yl}-(2-methoxy-ethyl)-amine;
{5-[3,4-Difluoro-2-(2-fluoro-4-iodo-phenylamino)-phenyl]-[1,3,4]oxadiazol-2-yl}-(2-phenoxy-ethyl)-amine;
{5-[3,4-Difluoro-2-(2-fluoro-4-iodo-phenylamino)-phenyl]-[1,3,4]oxadiazol-2-yl}-(2-pyridin-4-yl-ethyl)-amine;
Butyl-{5-[3,4-difluoro-2-(2-fluoro-4-iodo-phenylamino)-phenyl]-[1,3,4]oxadiazol-2-yl}-amine;
{5-[3,4-Difluoro-2-(2-fluoro-4-iodo-phenylamino)-phenyl]-[1,3,4]oxadiazol-2-yl}-(5-methyl-furan-2-ylmethyl)-amine;
{5-[3,4-Difluoro-2-(2-fluoro-4-iodo-phenylamino)-phenyl]-[1,3,4]oxadiazol-2-yl}-(2-thiophen-2-yl-ethyl)-amine;
{5-[3,4-Difluoro-2-(2-fluoro-4-iodo-phenylamino)-phenyl]-[1,3,4]oxadiazol-2-yl}-[2-(3-methoxy-phenyl)-ethyl]-amine;
{5-[3,4-Difluoro-2-(2-fluoro-4-iodo-phenylamino)-phenyl]-[1,3,4]oxadiazol-2-yl}-[2-(3,4-dimethoxy-phenyl)-ethyl]-amine;
or a pharmaceutically acceptable salt form thereof.

7. A compound of claim 1 selected from the group of:
{5-[3,4-Difluoro-2-(2-fluoro-4-iodo-phenylamino)-phenyl]-[1,3,4]oxadiazol-2-yl}-[2-(2,5-dimethoxy-phenyl)-ethyl]-amine;
{5-[3,4-Difluoro-2-(2-fluoro-4-iodo-phenylamino)-phenyl]-[1,3,4]oxadiazol-2-yl}-[2-(3,4-dimethoxy-phenyl)-ethyl]-methyl-amine;
{5-[3,4-Difluoro-2-(2-fluoro-4-iodo-phenylamino)-phenyl]-[1,3,4]oxadiazol-2-yl}-methyl-(2-pyridin-4-yl-ethyl)-amine;
{5-[3,4-Difluoro-2-(2-fluoro-4-iodo-phenylamino)-phenyl]-[1,3,4]oxadiazol-2-yl}-methyl-(2-pyridin-2-yl-ethyl)-amine;
{5-[3,4-Difluoro-2-(2-fluoro-4-iodo-phenylamino)-phenyl]-[1,3,4]oxadiazol-2-yl}-propyl-amine;
sec-Butyl-{5-[3,4-difluoro-2-(2-fluoro-4-iodo-phenylamino)-phenyl]-[1,3,4]oxadiazol-2-yl}-amine;
{5-[3,4-Difluoro-2-(2-fluoro-4-iodo-phenylamino)-phenyl]-[1,3,4]oxadiazol-2-yl}-(2,2-dimethyl-propyl)-amine;
{5-[3,4-Difluoro-2-(2-fluoro-4-iodo-phenylamino)-phenyl]-[1,3,4]oxadiazol-2-yl}-isobutyl-amine;
{5-[3,4-Difluoro-2-(2-fluoro-4-iodo-phenylamino)-phenyl]-[1,3,4]oxadiazol-2-yl}-(1,2,2-trimethyl-propyl)-amine;
{5-[3,4-Difluoro-2-(2-fluoro-4-iodo-phenylamino)-phenyl]-[1,3,4]oxadiazol-2-yl}-(2-methyl-butyl)-amine;
or a pharmaceutically acceptable salt form thereof.

8. A compound of claim 1 selected from the group of:
Bicyclo[2.2.1]hept-2-yl-{5-[3,4-difluoro-2-(2-fluoro-4-iodo-phenylamino)-phenyl]-[1,3,4]oxadiazol-2-yl}-amine;
{5-[3,4-Difluoro-2-(2-fluoro-4-iodo-phenylamino)-phenyl]-[1,3,4]oxadiazol-2-yl)}-[2-(1-methyl-1H-pyrrol-2-yl)-ethyl]-amine;
{5-[3,4-Difluoro-2-(2-fluoro-4-iodo-phenylamino)-phenyl]-[1,3,4]oxadiazol-2-yl}-[2-(1-methyl-1H-imidazol-4-yl)-ethyl]-amine;
{2,3-Difluoro-6-[5-(2-pyrrolidin-1-ylmethyl-pyrrolidin-1-yl)-[1,3,4]oxadiazol-2-yl]-phenyl}-(2-fluoro-4-iodo-phenyl)-amine;
{5-[3,4-Difluoro-2-(2-fluoro-4-iodo-phenylamino)-phenyl]-[1,3,4]oxadiazol-2-yl}-(2-methoxy-1-methyl-ethyl)-amine;
{5-[3,4-Difluoro-2-(2-fluoro-4-iodo-phenylamino)-phenyl]-[1,3,4]oxadiazol-2-yl}-(3-ethoxy-propyl)-amine;
3-{5-[3,4-Difluoro-2-(2-fluoro-4-iodo-phenylamino)-phenyl]-[1,3,4]oxadiazol-2-ylamino}-dihydro-furan-2-one;
3-5-[3,4-Difluoro-2-(2-fluoro-4-iodo-phenylamino)-phenyl]-[1,3,4]oxadiazol-2-ylamino}-propionic acid;
{5-[3,4-Difluoro-2-(2-fluoro-4-iodo-phenylamino)-phenyl]-[1,3,4]oxadiazol-2-yl}-[2-(2,6-dimethyl-phenoxy)-1-methyl-ethyl]-amine;
(1-{5-[3,4-Difluoro-2-(2-fluoro-4-iodo-phenylamino)-phenyl]-[1,3,4]oxadiazol-2-yl}-pyrrolidin-3-yl)-diethyl-amine;
or a pharmaceutically acceptable salt or ester form thereof.

9. A compound of claim 1 selected from the group of:
{2,3-Difluoro-6-[5-(4-methyl-[1,4]diazepan-1-yl)-[1,3,4]oxadiazol-2-yl]-phenyl}-(2-fluoro-4-iodo-phenyl)-amine;
{5-[3,4-Difluoro-2-(2-fluoro-4-iodo-phenylamino)-phenyl]-[1,3,4]oxadiazol-2-yl}-[2-(1-methyl-pyrrolidin-2-yl)-ethyl]-amine;
{5-[3,4-Difluoro-2-(2-fluoro-4-iodo-phenylamino)-phenyl]-[1,3,4]oxadiazol-2-yl}-(4-pyrrolidin-1-yl-butyl)-amine;
N*4*-{5-[3,4-Difluoro-2-(2-fluoro-4-iodo-phenylamino)-phenyl]-[1,3,4]oxadiazol-2-yl}-N*1*,N*1*-diethyl-pentane-1,4-diamine;
{5-[3,4-Difluoro-2-(2-fluoro-4-iodo-phenylamino)-phenyl]-[1,3,4]oxadiazol-2-yl}-(tetrahydro-furan-2-ylmethyl)-amine;
{5-[3,4-Difluoro-2-(2-fluoro-4-iodo-phenylamino)-phenyl]-[1,3,4]oxadiazol-2-yl}-bis-(2-methoxy-ethyl)-amine;
N-{5-[2-(4-Ethyl-2-fluoro-phenylamino)-3,4-difluoro-phenyl]-[1,3,4]oxadiazol-2-yl}-N′-methyl-ethane-1,2-diamine;
{5-[2-(4-Ethyl-2-fluoro-phenylamino)-3,4-difluoro-phenyl]-[1,3,4]oxadiazol-2-yl}-(2-morpholin-4-yl-ethyl)-amine;
2-{5-[2-(4-Ethyl-2-fluoro-phenylamino)-3,4-difluoro-phenyl]-[1,3,4]oxadiazol-2-ylamino}-ethanol;
Allyl-{5-[2-(4-ethyl-2-fluoro-phenylamino)-3,4-difluoro-phenyl]-[1,3,4]oxadiazol-2-yl}-amine;
or a pharmaceutically acceptable salt form thereof.

10. A compound of claim 1 selected from the group of:
{5-[3,4-Difluoro-2-(2-fluoro-4-iodo-phenylamino)-phenyl]-[1,3,4]thiadiazol-2-yl}-ethyl-amine;
{5-[2-(4-Ethyl-2-fluoro-phenylamino)-3,4-difluoro-phenyl]-1,3,4-oxadiazol-2-yl}-(3-pyrrolidin-1-yl-propyl)-amine;
N¹,N¹-Diethyl-N2-{5-[2-(4-ethyl-2-fluoro-phenylamino)-3,4-difluoro-phenyl]-1,3,4-oxadiazol-2-yl}-ethane-1,2-diamine;
{5-[2-(4-Ethyl-2-fluoro-phenylamino)-3,4-difluoro-phenyl]-1,3,4-oxadiazol-2-yl}-isopropyl-amine;
[2,3-Difluoro-6-(5-pyrrolidin-1-yl-1,3,4-oxadiazol-2-yl)-phenyl]-(4-ethyl-2-fluoro-phenyl)-amine;
N¹-{5-[2-(4-Ethyl-2-fluoro-phenylamino)-3,4-difluoro-phenyl]-1,3,4-oxadiazol-2-yl}-N>3_-methyl-propane-1,3-diamine;
{5-[2-(4-Ethyl-2-fluoro-phenylamino)-3,4-difluoro-phenyl]-1,3,4-oxadiazol-2-yl}-furan-2-ylmethyl-amine;
(2,2-Dimethoxy-ethyl)-{5-[2-(ethyl-2-fluoro-phenylamino)-3,4-difluoro-phenyl]-1,3,4-oxadiazol-2-yl}-amine;
{2,3-Difluoro-6-[5-(4-phenyl-piperazin-1-yl)-1,3,4-oxadiazol-2-yl]-phenyl}-(4-ethyl-2-fluoro-phenyl)-amine;
{5-[2-(4-Ethyl-2-fluoro-phenylamino)-3,4-difluoro-phenyl]-1,3,4-oxadiazol-2-yl}-(2-methoxy-ethyl)-amine;
or a pharmaceutically acceptable salt form thereof.

11. A compound of claim 1 selected from the group of:
6-{5-[2-(4-Ethyl-2-fluoro-phenylamino)-3,4-difluoro-phenyl]-1,3,4-oxadiazol-2-ylamino}-hexanenitrile;
(3,3-Diethoxy-propyl)-{5-[2-(4-ethyl-2-fluoro-phenylamino)-3,4-difluoro-phenyl]-1,3,4-oxadiazol-2-yl}-amine;
Diallyl-{5-[2-(4-ethyl-2-fluoro-phenylamino)-3,4-difluoro-phenyl]-1,3,4-oxadiazol-2-yl}-amine;
{5-[2-(4-Ethyl-2-fluoro-phenylamino)-3,4-difluoro-phenyl]-1,3,4-oxadiazol-2-yl}-(3-methylsulfanyl-propyl)-amine;
4-{5-[2-(4-Ethyl-2-fluoro-phenylamino)-3,4-difluoro-phenyl]-1,3,4-oxadiazol-2-ylamino}-isoxazolidin-3-one;
N¹-Ethyl-N¹-{5-[2-(4-ethyl-2-fluoro-phenylamino)-3,4-difluoro-phenyl]-1,3,4-oxadiazol-2-yl}-N²,N²-dimethyl-ethane-1,2-diamine;
{2,3-Difluoro-6-[5-(4-methyl-perhydro-1,4-diazepin-1-yl)-1,3,4-oxadiazol-2-yl]-phenyl}-(4-ethyl-2-fluoro-phenyl)-amine;
1-{5-[2-(4-Ethyl-2-fluoro-phenylamino)-3,4-difluoro-phenyl]-1,3,4-oxadiazol-2-yl}-piperidine-3-carboxylic acid diethylamide;
N-{5-[2-(4-Ethyl-2-fluoro-phenylamino)-3,4-difluoro-phenyl]-1,3,4-oxadiazol-2-yl}-O-(tetrahydro-pyran-2-yl)-hydroxylamine;
{5-[2-(4-Ethyl-2-fluoro-phenylamino)-3,4-difluoro-phenyl]-1,3,4-oxadiazol-2-yl}-(3-morpholin-4-yl-propyl)-amine;
or a pharmaceutically acceptable salt form thereof.

12. A compound of claim 1 selected from the group of:
{5-[2-(4-Ethyl-2-fluoro-phenylamino)-3,4-difluoro-phenyl]-1,3,4-oxadiazol-2-yl}-(4H-1,2,4-triazol-3-yl)-amine;
N¹-{5-[2-(4-Ethyl-2-fluoro-phenylamino)-3,4-difluoro-phenyl]-1,3,4-oxadiazol-2-yl}-N2-methyl-ethane-1,2-diamine;
N¹-{5-[2-(4-Ethyl-2-fluoro-phenylamino)-3,4-difluoro-phenyl]-1,3,4-oxadiazol-2-yl}-N1,N>3_,N>3_-trimethyl-propane-1,3-diamine;
{2,3-Difluoro-6-[5-(4-pyridin-2-yl-piperazin-1-yl)-1,3,4-oxadiazol-2-yl]-phenyl}-(4-ethyl-2-fluoro-phenyl)-amine;
N²-{5-[2-(4-Ethyl-2-fluoro-phenylamino)-3,4-difluoro-phenyl]-1,3,4-oxadiazol-2-yl}-N1,N1-dimethyl-propane-1,2-diamine;
N¹-(3-Dimethylamino-propyl)-N¹-5-[2-(4-ethyl-2-fluoro-phenylamino)-3,4-difluoro-phenyl]-1,3,4-oxadiazol-2-yl}-N>3_,N>3_-dimethyl-propane-1,3-diamine;
N¹-Cyclohexyl-N>3_-{5-[2-(4-ethyl-2-fluoro-phenylamino)-3,4-difluoro-phenyl]-1,3,4-oxadiazol-2-yl}-propane-1,3-diamine;
{5-[2-(4-Ethyl-2-fluoro-phenylamino)-3,4-difluoro-phenyl]-1,3,4-oxadiazol-2-yl}-(2-phenoxy-ethyl)-amine;
{5-[2-(4-Ethyl-2-fluoro-phenylamino)-3,4-difluoro-phenyl]-1,3,4-oxadiazol-2-yl}-(2-morpholin-4-yl-ethyl)-amine;
Cyclopropylmethyl-{5-[2-(4-ethyl-2-fluoro-phenylamino)-3,4-difluoro-phenyl]-1,3,4-oxadiazol-2-yl}-propyl-amine;
or a pharmaceutically acceptable salt form thereof.

13. A compound of claim 1 selected from the group of:
(4-Ethyl-2-fluoro-phenyl)-{6-[5-(4-ethyl-piperazin-1-yl)-1,3,4-oxadiazol-2-yl]-2,3-difluoro-phenyl}-amine;
{5-[2-(4-Ethyl-2-fluoro-phenylamino)-3,4-difluoro-phenyl]-1,3,4-oxadiazol-2-yl}-methyl-(1-methyl-pyrrolidin-3-yl)-amine;
N¹,N¹-Diethyl-N2-(2-{5-[2-(4-ethyl-2-fluoro-phenylamino)-3,4-difluoro-phenyl]-1,3,4-oxadiazol-2-ylamino}-ethyl)-ethane-1,2-diamine;
{5-[2-(4-Ethyl-2-fluoro-phenylamino)-3,4-difluoro-phenyl]-1,3,4-oxadiazol-2-yl}-thiophen-2-ylmethyl-amine;
{2,3-Difluoro-6-[5-(4-pyrrolidin-1-yl-piperidin-1-yl)-1,3,4-oxadiazol-2-yl]-phenyl}-(4-ethyl-2-fluoro-phenyl)-amine;
{5-[2-(4-Ethyl-2-fluoro-phenylamino)-3,4-difluoro-phenyl]-1,3,4-oxadiazol-2-yl}-(tetrahydro-pyran-2-ylmethyl)-amine;
{5-[2-(4-Ethyl-2-fluoro-phenylamino)-3,4-difluoro-phenyl]-1,3,4-oxadiazol-2-yl}-(2-pyridin-4-yl-ethyl)-amine;
{6-[5-(4-Benzyl-perhydro-1,4-diazepin-1-yl)-1,3,4-oxadiazol-2-yl]-2,3-difluoro-phenyl}-(4-ethyl-2-fluoro-phenyl)-amine;
{5-[2-(4-Ethyl-2-fluoro-phenylamino)-3,4-difluoro-phenyl]-1,3,4-oxadiazol-2-yl}-pyridin-2-ylmethyl-amine;
{5-[2-(4-Ethyl-2-fluoro-phenylamino)-3,4-difluoro-phenyl]-1,3,4-oxadiazol-2-yl}-(5-methyl-furan-2-ylmethyl)-amine;
or a pharmaceutically acceptable salt form thereof.

14. A compound of claim 1 selected from the group of:
(2-Ethoxy-benzyl)-{5-[2-(4-ethyl-2-fluoro-phenylamino)-3,4-difluoro-phenyl]-1,3,4-oxadiazol-2-yl}-amine;
{2,3-Difluoro-6-[5-(4-methyl-piperazin-1-yl)-1,3,4-oxadiazol-2-yl]-phenyl}-(4-ethyl-2-fluoro-phenyl)-amine;
{5-[2-(4-Ethyl-2-fluoro-phenylamino)-3,4-difluoro-phenyl]-1,3,4-oxadiazol-2-yl}-pyridin-3-ylmethyl-amine;
{5-[2-(4-Ethyl-2-fluoro-phenylamino)-3,4-difluoro-phenyl]-1,3,4-oxadiazol-2-yl}-(2-thiophen-2-yl-ethyl)-amine;
{5-[2-(4-Ethyl-2-fluoro-phenylamino)-3,4-difluoro-phenyl]-1,3,4-oxadiazol-2-yl}-(2-pyridin-3-yl-ethyl)-amine;
{5-[2-(4-Ethyl-2-fluoro-phenylamino)-3,4-difluoro-phenyl]-1,3,4-oxadiazol-2-yl}-pyridin-4-ylmethyl-amine;
{5-[2-(4-Ethyl-2-fluoro-phenylamino)-3,4-difluoro-phenyl]-1,3,4-oxadiazol-2-yl}-(2-pyridin-2-yl-ethyl)-amine;
N-(2-{5-[2-(4-Ethyl-2-fluoro-phenylamino)-3,4-difluoro-phenyl]-1,3,4-oxadiazol-2-ylamino}-ethyl)-acetamide;
{5-[2-(4-Ethyl-2-fluoro-phenylamino)-3,4-difluoro-phenyl]-1,3,4-oxadiazol-2-yl}-(6-methoxy-pyridin-3-yl)-amine;
{2,3-Difluoro-6-[5-(2-pyridin-4-yl-pyrrolidin-1-yl)-1,3,4-oxadiazol-2-yl]-phenyl}-(4-ethyl-2-fluoro-phenyl)-amine;
or a pharmaceutically acceptable salt form thereof.

15. A compound of claim 1 selected from the group of:
{5-[2-(4-Ethyl-2-fluoro-phenylamino)-3,4-difluoro-phenyl]-1,3,4-oxadiazol-2-yl}-(2-methanesulfonyl-ethyl)-methyl-amine;
N²-{5-[2-(4-Ethyl-2-fluoro-phenylamino)-3,4-difluoro-phenyl]-1,3,4-oxadiazol-2-yl}-N¹,N¹-dimethyl-1-phenyl-ethane-1,2-diamine;
{2,3-Difluoro-6-[5-(4-pyridin-2-ylmethyl-piperazin-1-yl)-1,3,4-oxadiazol-2-yl]-phenyl}-(4-ethyl-2-fluoro-phenyl)-amine;
{5-[2-(4-Ethyl-2-fluoro-phenylamino)-3,4-difluoro-phenyl]-1,3,4-oxadiazol-2-yl}-(2-morpholin-4-yl-1-phenyl-ethyl)-amine;
{2,3-Difluoro-6-[5-(3-pyridin-4-yl-pyrrolidin-1-yl)-1,3,4-oxadiazol-2-yl]-phenyl}-(4-ethyl-2-fluoro-phenyl)-amine;
{5-[2-(4-Ethyl-2-fluoro-phenylamino)-3,4-difluoro-phenyl]-1,3,4-oxadiazol-2-yl}-methyl-(2-pyridin-4-yl-ethyl)-amine;
{5-[2-(4-Ethyl-2-fluoro-phenylamino)-3,4-difluoro-phenyl]-1,3,4-oxadiazol-2-yl}-methyl-(2-pyridin-2-yl-ethyl)-amine;
{5-[2-(4-Ethyl-2-fluoro-phenylamino)-3,4-difluoro-phenyl]-1,3,4-oxadiazol-2-yl}-(2-morpholin-4-yl-2-pyridin-3-yl-ethyl)-amine;
N¹-{5-[2-(4-Ethyl-2-fluoro-phenylamino)-3,4-difluoro-phenyl]-1,3,4-oxadiazol-2-yl}-N²,N²-dimethyl-1-phenyl-ethane-1,2-diamine;
{5-[2-(4-Ethyl-2-fluoro-phenylamino)-3,4-difluoro-phenyl]-1,3,4-oxadiazol-2-yl}-(3-imidazol-1-yl-propyl)-amine;
or a pharmaceutically acceptable salt form thereof.

16. A compound of claim 1 selected from the group of:
{5-[2-(4-Ethyl-2-fluoro-phenylamino)-3,4-difluoro-phenyl]-1,3,4-oxadiazol-2-yl}-methyl-(tetrahydro-pyran-2-ylmethyl)-amine;
{5-[2-(4-Ethyl-2-fluoro-phenylamino)-3,4-difluoro-phenyl]-1,3,4-oxadiazol-2-yl}-(2-furan-2-yl-ethyl)-amine;
{5-[2-(4-Ethyl-2-fluoro-phenylamino)-3,4-difluoro-phenyl]-1,3,4-oxadiazol-2-yl}-(2-thiophen-2-yl-thiazol-4-ylmethyl)-amine;
{5-[2-(4-Ethyl-2-fluoro-phenylamino)-3,4-difluoro-phenyl]-1,3,4-oxadiazol-2-yl}-(5-methyl-isoxazol-3-ylmethyl)-amine;
{5-[2-(4-Ethyl-2-fluoro-phenylamino)-3,4-difluoro-phenyl]-1,3,4-oxadiazol-2-yl}-[2-(4-methyl-1H-imidazol-2-yl)-ethyl]-amine;
{5-[2-(4-Ethyl-2-fluoro-phenylamino)-3,4-difluoro-phenyl]-1,3,4-oxadiazol-2-yl}-[2-(5-pyridin-4-yl-2H-1,2,4-triazol-3-yl)-ethyl]-amine;
{5-[2-(4-Ethyl-2-fluoro-phenylamino)-3,4-difluoro-phenyl]-1,3,4-oxadiazol-2-yl}-(1-methyl-2-thiophen-3-yl-ethyl)-amine;
{5-[2-(4-Ethyl-2-fluoro-phenylamino)-3,4-difluoro-phenyl]-1,3,4-oxadiazol-2-yl}-(1H-tetrazol-5-ylmethyl)-amine;
{5-[2-(4-Ethyl-2-fluoro-phenylamino)-3,4-difluoro-phenyl]-1,3,4-oxadiazol-2-yl}-(3-phenyl-1H-pyrazol-4-ylmethyl)-amine; or
2-{5-[2-(4-ethynyl-2-fluoro-phenylamino)-3,4-difluoro-phenyl]-[1,3,4]oxadiazol-2-ylamino}-ethanol
or a pharmaceutically acceptable salt form thereof.

17. A compound of claim 1 selected from the group of:
[2,3-Difluoro-6-(5-methyl-4H-[1,2,4]triazol-3-yl)-phenyl]-(4-ethyl-2-fluoro-phenyl)-amine;
(4-Ethyl-2-fluoro-phenyl )-[6-(5-ethyl-[1,3,4]oxadiazol-2-yl)-2,3-difluoro-phenyl]-amine;
[6-(5-Ethyl-[1,3,4]oxadiazol-2-yl)-2,3-difluoro-phenyl]-(2-fluoro-4-iodo-phenyl)-amine;
[6-(5-Ethyl-[1,3,4]oxadiazol-2-yl)-2,3-difluoro-phenyl]-(4-ethynyl-2-fluoro-phenyl)-amine;
{6-[5-(3-Bromo-propyl)-[1,3,4]oxadiazol-2-yl]-2,3-difluoro-phenyl}-(4-ethyl-2-fluoro-phenyl)-amine;
{2,3-Difluoro-6-[5-(3-morpholin-4-yl-propyl)-[1,3,4]oxadiazol-2-yl]-phenyl}-(4-ethyl-2-fluoro-phenyl)-amine;
3-(4-{2,3-Difluoro-6-[5-(2-morpholin-4-yl-ethylamino)-[1,3,4]oxadiazol-2-yl]-phenylamino}-3-fluoro-phenyl)-propan-1-ol;
{5-[2-(4-Ethynyl-2-fluoro-phenylamino)-3,4-difluoro-phenyl]-[1,3,4]oxadiazol-2-yl}-(2-morpholin-4-yl-ethyl)-amine;
1-(4-{2,3-Difluoro-6-[5-(2-morpholin-4-yl-ethylamino)-[1,3,4]oxadiazol-2-yl]-phenylamino}-3-fluoro-phenyl)-ethanone;
3-{5-[3,4-Difluoro-2-(2-fluoro-4-iodo-phenylamino)-phenyl]-[1,3,4]oxadiazol-2-ylamino}-propane-1,2 (R)-diol;
3-{5-[2-(4-Ethynyl-2-fluoro-phenylamino)-3,4-difluoro-phenyl]-[1,3,4]oxadiazol-2-ylamino}-propane-1,2(R)-diol;
3-{5-[2-(4-Ethyl-2-fluoro-phenylamino)-3,4-difluoro-phenyl]-[1,3,4]oxadiazol-2-ylamino}-propane-1,2(R)-diol;
3-{5-[3,4-Difluoro-2-(2-fluoro-4-iodo-phenylamino)-phenyl]-[1,3,4]oxadiazol-2-ylamino}-propane-1,2 (S)-diol;
2-{5-[3,4-Difluoro-2-(2-fluoro-4-iodo-phenylamino)-phenyl]-[1,3,4]oxadiazol-2-ylamino}-propane-1,3-diol
2-{5-[2-(4-Ethynyl-2-fluoro-phenylamino)-3,4-difluoro-phenyl]-[1,3,4]oxadiazol-2-ylamino}-propane-1,3-diol;
{5-[3,4-Difluoro-2-(2-fluoro-4-vinyl-phenylamino)-phenyl]-[1,3,4]oxadiazol-2-yl}-(2-morpholin-4-yl-ethyl) -amine;
2-(4-{2,3-difluoro-6-[5-(2-morpholin-4-yl-ethylamino)-[1,3,4]oxadiazol-2-yl]-phenylamino}-3-fluoro-phenyl)-ethanol;
2-{5-[3,4-Difluoro-2-(2-fluoro-4-iodo-phenylamino)-phenyl]-[1,3,4]oxadiazol-2-ylsulfanyl}-ethanol;
[6-(5-Allylsulfanyl-[1,3,4]oxadiazol-2-yl)-2,3-difluoro-phenyl]-(2-fluoro-4-iodo-phenyl)-amine;
2-{5-[3,4-Difluoro-2-(2-fluoro-4-methylsulfanyl-phenylamino)-phenyl]-[1,3,4]oxadiazol-2-ylamino}-propane-1,3-diol;
2-{5-[2-(4-Ethyl-2-fluoro-phenylamino)-3,4-difluoro-phenyl]-[1,3,4]oxadiazol-2-ylamino}-propane-1,3-diol;
3-{5-[3,4-Difluoro-2-(2-fluoro-4-methylsulfanyl-phenylamino)-phenyl]-[1,3,4]oxadiazol-2-ylamino}-propane-1,2(R)-diol;
3-{5-[3,4-Difluoro-2-(2-fluoro-4-methylsulfanyl-phenylamino)-phenyl]-[1,3,4]oxadiazol-2-ylamino}-propane-1,2(S)-diol;
3-{5-[2-(4-Ethyl-2-fluoro-phenylamino)-3,4-difluoro-phenyl]-[1,3,4]oxadiazol-2-ylamino}-propane-1,2(S)-diol;
3-{5-[2-(4-Ethynyl-2-fluoro-phenylamino)-3,4-difluoro-phenyl]-[1,3,4]oxadiazol-2-ylamino}-propane-1,2(S)-diol;
2-{5-[3,4-Difluoro-2-(2-fluoro-4-methylsulfanyl-phenylamino)-phenyl]-[1,3,4]oxadiazol-2-ylamino}-ethanol;
2-(4-{2,3-difluoro-6-[5-(2-methylamino-ethylamino)-1[1,3,4]oxadiazol-2-yl]-phenylamino}-3-fluoro-phenyl)-ethanol; or
N-(2-{5-[2-(4-Ethyl-2-fluoro-phenylamino)-3,4-difluoro-phenyl]-[1,3,4]oxadiazol-2-ylamino}-ethyl)-N-methyl-acetamide;
Acetic acid 2-{5-[2-(4-ethyl-2-fluoro-phenylamino)-3,4-difluoro-phenyl]-[1,3,4]oxadiazol-2-ylamino}-ethyl ester
or a pharmaceutically acceptable salt form thereof.

18. A pharmaceutical composition comprising a pharmaceutically effective amount of a compound of claim 1, or a pharmaceutically acceptable salt or ester form thereof, and a pharmaceutically acceptable carrier.

19. A method of treating a proliferative disease in a patient in need thereof comprising administering a therapeutically effective amount of a compound of claim 1.

20. A method for treating cancer in a patient in need thereof comprising administering a therapeutically effective amount of a compound of claim 1 in combination with radiation therapy.

21. A method of claim 19 wherein the cancer is brain, breast, lung, ovarian, pancreatic, prostate, renal, or colorectal cancer.

22. A method of treating neuropathic pain in a patient in need thereof comprising administering a therapeutically effective amount of a compound of claim 1.

23. A method of treating chronic pain in a patient in need thereof comprising administering a therapeutically effective amount of a compound of claim 1.
